(19) United States
(12) Patent Application Publication
(10) Pub. No.: US 2017/0254822 A1
(43) Pub. Date: Sep. 7, 2017

(54) METHODS AND KITS FOR DETECTING AND DIAGNOSING NEUROTRAUMA

(71) Applicant: Roger A. SABBADINI, Bend, OR (US)

(72) Inventor: Roger A. SABBADINI, Bend, OR (US)

(21) Appl. No.: 15/331,880

(22) Filed: Oct. 23, 2016

Related U.S. Application Data
(63) Continuation of application No. 13/778,917, filed on Feb. 27, 2013, now abandoned.
(60) Provisional application No. 61/605,076, filed on Feb. 29, 2012.

(51) Int. Cl. G01N 33/92; G01N 33/68

(52) U.S. Cl. CPC G01N 33/92; G01N 2405/04; G01N 2800/2871; G01N 33/6896

(57) ABSTRACT

Methods and kits for detecting and diagnosing neurotrauma (e.g., traumatic brain injury, stroke, or spinal cord injury) are provided. These methods rely on the determination of lysophosphatidic acid (LPA) and/or LPA metabolite levels in patient samples following suspected injury.

### RELATED APPLICATIONS

This patent application is a continuation of U.S. patent application Ser. No. 13/778,917 (attorney docket no. LPT-3230-UT), filed 27 Feb. 2013, which claims the benefit of and priority to U.S. provisional patent application Ser. No. 61/605,076, filed 29 Feb. 2012 (attorney docket number LPT-3230-PV), the contents of which are hereby incorporated by reference in their entirety for any and all purposes.

### TECHNICAL FIELD

The present invention relates to methods of detecting and/or diagnosing neurotrauma. These methods are based on the novel observation that levels of lysophosphatidic acid (LPA) and certain LPA metabolites rise following neurotrauma, e.g., spinal cord injury, stroke, traumatic brain injury (TBI), etc. The methods include determining levels of LPA and metabolites in a biological sample, e.g., a biological fluid or tissue sample. LPA or LPA metabolite levels may be determined by immunologic methods, e.g., ELISA, immunohistochemical methods; lateral flow immunoassay diagnostics, enzymatic methods, mass spectrometry, or other methods known in the art, whether now existing or later developed.

The present invention also relates to kits for detecting neurotrauma, which kits include agents that bind LPA and its variants, particularly to monoclonal antibodies, antigen-binding antibody fragments, and antibody derivatives specifically reactive to LPA or an LPA metabolite under physiological conditions. Such kits can be used, e.g., in the diagnosis of diseases and conditions associated with aberrant levels of LPA, the monitoring of progression of such diseases, and in the monitoring and evaluation of treatment efficacy for such diseases and conditions as well as companion diagnostic tests to identify targeted patient populations who might benefit from anti-LPA therapy. Screening of subjects suspected of neurotrauma could also aid in decisions for hospital admission and treatment regimens including surgery and other invasive or costly procedures.

LPA is a bioactive lipid mediating multiple cellular responses including proliferation, differentiation, angiogenesis, motility, and protection from apoptosis in a variety of cell types. LPA is involved in the establishment and progression of cancer by providing a pro-growth tumor microenvironment and promoting angiogenesis. In addition, LPA has been implicated in fibrosis, ocular diseases such as macular degeneration, neurotrauma, and pain-related disorders.

Lpath, Inc. owns or otherwise controls patent rights that cover, among others, a family of high-affinity, specific monoclonal antibodies to LPA, one of which is known as Lpathomab. The efficacy of Lpathomab in various animal models of cancer, fibrosis, and ocular disorders highlights the utility of this class of anti-LPA antibodies (and molecules derived therefrom), for example, in the treatment of malignancies, angiogenesis, and fibrosis-related disorders. Lpathomab has also been shown to be effective in treating neurotrauma, e.g., spinal cord injury and traumatic brain injury, and in the reduction of pain, including neuropathic pain. In addition to therapeutic advantages, antibodies to LPA also have the advantage of being useful for the detection of LPA. As will be described in more detail below, levels of LPA are elevated or aberrant in a number of diseases or conditions. This fact, combined with the high-affinity binding anti-LPA antibodies that Lpath has developed, has led to the development of kits and methods for detection of LPA and diagnosis of diseases associated with aberrant levels of LPA. In addition, sensitive measurement of LPA levels in clinical samples allows the monitoring of the efficacy of treatment of disease, e.g., in blood, blood fractions (e.g., serum, plasma, etc.), urine, and/or cerebrospinal fluid (CSF). Thus companion diagnostics are envisioned, i.e., an antibody therapeutic for treatment of neurotrauma and an antibody method and/or kit for detection of LPA levels during such treatment.

### BACKGROUND OF THE INVENTION

1. Introduction.

The following description includes information that may be useful in understanding the present invention. It is not an admission that any of the information provided herein, or any publication specifically or implicitly referenced herein, is prior art, or even particularly relevant, to the presently claimed invention.

2. Background

### A. Neurotrauma

Neurotrauma refers to injury to the central nervous system, whether through injury, hemorrhage or disease. Major types of neurotrauma include spinal cord injury (SCI), traumatic brain injury (TBI), stroke (ischemic or hemorrhagic). CNS injury is the type of injury most likely to result in death or lifelong disability.

i. Traumatic Brain Injury (TBI)

TBI is a disruption of function in the brain that results from a blow or jolt to the head or penetrating head injury. There are more than 1.5 million TBIs per year in the US, with 125,000 of these resulting in permanent disability. Common causes of TBI include falls, vehicle-related collisions, violence, sports injuries and combat injuries, including explosive blasts. TBI is also the leading cause of military casualties in the field and a leading source of long-term rehabilitation problems suffered by veterans. When not fatal (22% of moderate and 35% of severe TBI patients die within the first year following injury), TBI can result in permanent and severe physical, cognitive, and behavioral impairments, leaving sufferers in need of long term healthcare. Currently, there are no FDA-approved drugs targeting TBI.

TBI is heterogeneous in its cause and can be seen as a two-step event: 1) a primary injury, which can be focal or diffuse, caused by mechanical impact, that results in primary pathological events such as hemorrhage and ischemia, tearing of tissue and axonal injuries; 2) a secondary injury such as diffuse inflammation, cell death and gliosis, which is a consequence of the primary one. This secondary injury starts immediately after injury and can continue for weeks, and is thought to involve an active inhibition of neural stem cell activity. Collectively, these events lead to neurodegeneration.

A large fraction of TBI are mild, and thus may go undiagnosed immediately after injury. Because there is no single TBI symptom or pattern of symptoms that characterize mild TBI, for example, a rapid screening test, ideally one (such as a kit described herein) that can be used in the field, an emergency room or in a rescue vehicle. Undiagnosed and untreated TBI presents a risk because some signs and symptoms may be delayed from days to months after injury, and may have significant impact on the patient's physical, emotional, behavioral, social, or family status if untreated, and may result in a functional impairment. Because secondary damage from the injury continues after the initial impact, early treatment (and thus rapid diagnosis), particularly point-of-care treatment, is desirable. An ideal therapy for TBI would reduce the injury infarct size as well as limiting the secondary inflammatory responses.

ii. Spinal Cord Injury (SCI)

SCI usually begins with a sudden, traumatic blow to the spine that fractures or dislocates vertebrae, or with an injury that transects the spinal cord. Common causes of SCI include motor vehicle accidents, falls, acts of violence, sports and recreation injuries, and certain diseases that impact the spine. Spinal cord injuries occur in combat situations as well, and spinal cord injuries account for about five percent (5%) of casualties in the current Iraq and Afghanistan conflicts. The damage begins at the moment of injury when the cord is directly damaged, or when surrounding bone, discs, or ligaments bruise or tear spinal cord tissue, causing destruction of axons, which are the long extensions of nerve cells that carry signals up and down the spinal cord between the brain and the rest of the body. An injury to the spinal cord can damage a few, many, or most of these axons, and the extent of the resulting paralysis and loss of sensation is variable as a result. Improved emergency care and aggressive treatment and rehabilitation can help minimize damage to the nervous system and even restore limited abilities. Surgery may be needed to relieve compression of the spinal cord and to repair fractures. The steroid drug methylprednisolone appears to reduce the damage to nerve cells if it is given within the first 8 hours after injury. In addition to paralysis and loss of sensation, SCI is often accompanied by respiratory problems (with higher levels of injury often requiring ventilator support), chronic pain and bladder and bowel dysfunction, and an increased susceptibility to heart problems.

iii. Stroke

A stroke is a sudden interruption of blood flow to the brain caused by hemorrhage (bleeding) in the brain, usually caused by a ruptured blood vessel, or by a loss of blood flow (ischemia) to an area of the brain, such as may be caused by a blood clot lodging in an artery to a portion of the brain. Ischemic strokes account for the vast majority of stroke. Strokes may cause sudden weakness, loss of sensation, or difficulty with speaking, seeing, or walking. Symptoms vary according to the location and extent of the interruption in blood flow and resulting tissue damage. Stroke is the third leading cause of death and the leading cause of serious, long-term disability in the United States. Stroke is typically determined by physical examination, particularly by imaging such as CT scan, MRI scan etc. Stroke cannot currently be diagnosed by blood test(s). However blood tests may be done to further understand the medical condition that has lead to stroke symptoms. Lumbar puncture is often performed if a stroke due to subarachnoid hemorrhage is suspected, or if other CNS conditions such as meningitis are suspected.

### B. Bioactive Signaling Lipids

Certain lipids and their derivatives are now recognized as important targets for medical research, not as just simple structural elements in cell membranes or as a source of energy for β-oxidation, glycolysis or other metabolic processes. In particular, certain lipids function as signaling mediators important in animal and human disease. Although most of the lipids of the plasma membrane play an exclusively structural role, a small proportion of them are involved in relaying extracellular stimuli into cells. These lipids are referred to as “bioactive lipids” or, alternatively, “bioactive signaling lipids.” “Lipid signaling” refers to any of a number of cellular signal transduction pathways that use cell membrane lipids as second messengers, as well as referring to direct interaction of a lipid signaling molecule with its own specific receptor. Lipid signaling pathways are activated by a variety of extracellular stimuli, ranging from growth factors to inflammatory cytokines, and regulate cell fate decisions such as apoptosis, differentiation and proliferation. Research into bioactive lipid signaling is an area of intense scientific investigation as more and more bioactive lipids are identified and their actions characterized.

Examples of bioactive lipids include the eicosanoids (including the cannabinoids, leukotrienes, prostaglandins, lipoxins, epoxyeicosatrienoic acids, and isoeicosanoids), non-eicosanoid cannabinoid mediators, phospholipids and their derivatives such as phosphatidic acid (PA) and phosphatidylglycerol (PG), platelet activating factor (PAF) and cardiolipins as well as lysophospholipids such as lysophosphatidyl choline (LPC) and various lysophosphatidic acids (LPA). Bioactive signaling lipids also include the sphingolipids such as sphingomyelin, ceramide, ceramide-1-phosphate, sphingosine, sphingosylphosphoryl choline, sphinganine, sphinganine-1-phosphate (dihydro-S1P) and sphingosine-1-phosphate. Sphingolipids and their derivatives represent a group of extracellular and intracellular signaling molecules with pleiotropic effects on important cellular processes. Other examples of bioactive signaling lipids include phosphatidylinositol (PI), phosphatidylethanolamine (PEA), diacylglyceride (DG), sulfatides, gangliosides, and cerebrosides.

1. Lysolipids

Lysophospholipids (LPLs), also known as lysolipids, are low molecular weight (typically less than about 500 dalton) lipids that contain a single hydrocarbon backbone and a polar head group containing a phosphate group. Some lysolipids are bioactive signaling lipids. Two particular examples of medically important bioactive lysolipids are LPA (glycerol backbone) and S1P (sphingoid backbone). The structures of selected LPAs, S1P, and dihydro S1P are presented below.

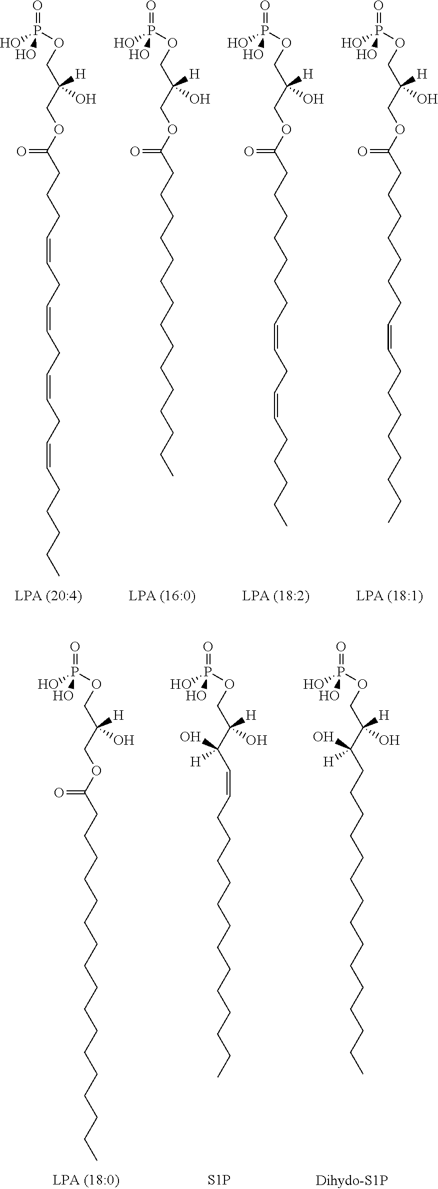

The structural backbone of LPA is derived from glycerol-based phospholipids such as phosphatidylcholine (PC) or phosphatidic acid (PA). In the case of lysosphingolipids such as S1P, the fatty acid of the ceramide backbone is missing. The structural backbone of S1P, dihydro S1P (DHS1P), and sphingosylphosphorylcholine (SPC) is based on sphingosine, which is derived from sphingomyelin.

LPA and S1P regulate various cellular signaling pathways by binding to the same class of multiple transmembrane domain G protein-coupled (GPCR) receptors. The S1P receptors are designated as S1P1, S1P2, S1P3, S1P4, and S1P5 (formerly EDG-1, EDG-5/AGR16, EDG-3, EDG-6 and EDG-8) and the LPA receptors designated as LPA1, LPA2, and LPA3 (formerly, EDG-2, EDG-4, and EDG-7). A fourth LPA receptor of this family has been identified for LPA (LPA4), and other putative receptors for these lysophospholipids have also been reported.

LPA and S1P have been shown to play a role in the immune response through modulation of immune-related cells such as T- and B-lymphocytes. These lipids promote T-cell migration to sites of immune response and regulate proliferation of T cells as well as secretion of various cytokines. In particular, S1P is thought to control egress of lymphocytes into the peripheral circulation. Thus, agents which bind LPA and S1P are believed to be useful in methods for decreasing an undesired, excessive or aberrant immune response, and for treating diseases and conditions, including certain hematological cancers and autoimmune disorders that are associated with an undesired, excessive or aberrant involvement of lymphocytes and or an aberrant immune response.

a. Lysophosphatic Acid (LPA)

Lysophosphatidic acid (mono-acylglycerol-3-phosphate, <500 Dalton) consists of a single hydrocarbon backbone and a polar head group containing a phosphate group. LPA is not a single molecular entity but a collection of endogenous structural variants with fatty acids of varied lengths and degrees of saturation. Thus, when used herein, “LPA” refers to the set of physiologically relevant bioactive LPA variants, unless stated otherwise. According to standard nomenclature for the isoforms, the number 18:2, for example, indicates that the LPA isoform bears an 18-carbon fatty acid having 2 double bonds. Biologically relevant variants of LPA include 18:2, 18:1, 18:0, 16:0, and 20:4. LPA species with both saturated fatty acids (16:0 and 18:0) and unsaturated fatty acids (16:1, 18:1, 18:2, and 20:4) have been detected in serum and plasma. The 16:0, 18:1, 18:2, and 20:4 LPA isoforms are the predominant species in blood.

Detectable levels of LPA have been found in various body fluids, including serum, plasma, saliva, follicular fluid, inflammatory fluids, some malignant effusions and cerebrospinal fluid [reviewed in Aoki, et al. [(2008) Biochim Biophys Acta 1781:513-8]. A broad range of cell types are known to produce LPA, including platelets, post-mitotic neurons, astrocytes, erythrocytes, adipocytes, and various cancer cells. LPA species with both saturated (16:0 and 18:0) and unsaturated fatty acids (16:1, 18:1, 18:2, and 20:4) have been identified in biological fluids with 16:0, 18:2, 18:1, 18:0 and 20:4 LPA being the predominant species (Aoki, et al., 2008).

LPA can be produced from various precursors, including glycerol 3-phosphate, phosphatidic acid and various lysophospholipids. In de novo LPA synthesis, which is important for glycerophospholipids and triglyceride synthesis, LPA is synthesized by the acylation of glycerol-3-phosphate by glycerol 3-phosphate acetyltransferase in the endoplasmic reticulum. This route is likely to occur by demand and not regulated by cell signaling.

The main source of LPA in serum and plasma is due to the activity of autotaxin (ATX, lyso-phospholipase D), which generates LPA by hydrolysis of various lysophospholipids (LPLs) such as lysophosphatidylcholine (LPC), lysophosphatidylethanolamine, and lysophosphatidylserine released from activated platelets. ATX is found in diverse biological fluids such as the cerebrospinal fluid, plasma, and semen; and is the main enzyme responsible for LPA presence in human plasma [Sato (2005), J Neurochem 92:904-14; Tokumura, et al. (2002), Biochim Biophys Acta. 1582:18-25].

LPA can also be synthesized extracellularly through the deacylation of phosphatidic acid by secreted phospholipases A1 and A2, and a similar mechanism involving intracellular phospholipases A1 and A2 would be responsible for LPA levels in platelets (Aoki, et al., 2008). Finally, LPA could also arise from phosphorylation of monoacylglycerol by monoacylglycerol kinase in the mitochondria.

Under physiological conditions, LPA is present in small amounts in most cell types since it plays a role as an intermediate molecule in the early steps of phospholipid biosynthesis. In normal physiology, LPA appears to regulate its own biosynthesis through auto inhibition of ATX; thus, maintaining the extracellular LPA level in plasma in low/basal level of approximately 0.1-1 μM in serum [Baker, et al. (2001), Anal Biochem 292:287-95]. Interestingly, elevated levels of LPA are observed in certain pathological states such as atherosclerosis [Siess, et al. (1999), Proc Natl Acad Sci USA. 96(12):6931-6], ovarian cancer [Eder, et al. (2000), Clin Cancer Res. 6:2482-91] and injured cornea [Liliom, et al. (1988), Am J Physiol. 274:C1065-74] and are believed to reach levels up to 10 μM in a cerebral hemorrhagic injury model in which blood is injected intrathecally in piglets [Tigyi, et al. (1995), Am J. Physiol. 268:H2048-2055]. Furthermore, the increased level of ATX expression is associated with cancer and tumor aggressiveness [Mills, G B and Moolenaar, W H (2003), Nat Rev. Cancer 3(8):582-91].

In the adult rat brain, LPA is found with values of ˜1-14 nmol/g although a higher value of ˜80 ng/ml was first reported [Aaltonen, et al. (2010), J Chromatogr B Analyt Technol Biomed Life Sci. 878:1145-52; Sugiura, et al. (1999), Biochim Biophys Acta. 1440:194-204] with its highest level in the brainstem and midbrain, at intermediate levels in the thalamus and at the lowest in the cortex and cerebellum, hence suggesting a variation in LPA synthesis and physiological role within the adult brain, such as involvement in the descending regulatory pathways for pain. LPA is also present in the cerebrospinal fluid of rats and dogs [Sato, et al. (2005), J Neurochem. 92(4):904-14] and ATX is present in plexus choroids of the rat and mouse brains [Fuss, et al. (1997), J Neurosci 17(23): 9095-9103; Narita, et al. (1994), J Biol Chem 269(45):28235-42].

Following injury, LPA is synthesized in the mouse spinal cord in a model of sciatic nerve ligation [Ma, et al. (2010), J Pharmacol Exp Ther. 333(2):540-6] and LPA-like activity is increased in the cerebrospinal fluid following intrathecal injection of autologous blood in newborn pigs [Tigyi, et al. (1995), Am J. Physiol. 268:H2048-2055]. Normally undetectable, levels of ATX increase in astrocytes neighboring a lesion of the adult rat brain [Savaskan, et al. (2007), Cell Mol Life Sci. 64(2):230-43]. In humans, the presence of ATX in cerebrospinal fluid has been demonstrated in multiple sclerosis patients [Hammack, et al. (2004), Mult Scler. 10(3):245-60] and higher levels of LPA in human plasma are speculated to predict silent brain infarction in patients with nonvalvular atrial fibrillation [Li, et al. (2010), Int J Mol Sci. 11(10):3988-98]. Further, in human cerebrospinal fluid from traumatic brain injury (TBI) patients, [Farias, et al (2011), J Trauma. 2011 71(5):1211-8], increased levels of arachidonic acid, a lipid generated from the hydrolysis of phosphatidic acid into LPA and arachidonic acid, have been described. Consistently, LPA levels also increase in post sciatic nerve injury in mice, reaching approximately a hundred times higher compared to the basal level of normal tissue [Ma, et al. (2010), J Pharm Exp Therapeut. 333: 540-546]. In human brains following injury, LPA₁ was found to be expressed by reactive astrocytes and LPA₂ by ependymal cells lining the lateral ventricle. Interestingly, LPA₂ mRNA was upregulated and ATX mRNA downregulated in the cortex of these injured human brains. Frugier, et al. (2011), Cell Mol Neurobiol. 31(4):569-77.

ATX expression is significantly upregulated in reactive astrocytes adjacent to the lesion site (Savaskan et al., 2007) and PLA₂ activity is increased in several types of CNS injury, such as closed head injury [Shohami et al. (1989), J Neurochem 53(5):1541-6, abstract], brain ischemia [Rordorf, et al. (1991), J Neurosci 11(6):1829-36] or spinal cord injury (Ma, et al., 2010), suggesting a role of PLA₂ in facilitating the LPA production post injury. Indeed, either the knockdown of the ATX gene or the pharmacological inhibition of PLA₂ significantly attenuates LPA production post injury and neuropathic pain (Ma, et al., 2010).

Until now, no direct measurements have been made of LPA in the cerebrospinal fluid after neurotrauma in human patients. The breakdown of the BBB is believed to allow the entrance of hematopoietic cells, including platelets, from the bloodstream to the injury site, and may allow higher levels of LPA to be present through its release by activated platelets.

LPA has long been known as a precursor of phospholipid biosynthesis in both eukaryotic and prokaryotic cells, but LPA has emerged only recently as a signaling molecule that are rapidly produced and released by activated cells, notably platelets, to influence target cells by acting on specific cell-surface receptor. Besides being synthesized and processed to more complex phospholipids in the endoplasmic reticulum, LPA can be generated through the hydrolysis of pre-existing phospholipids following cell activation; for example, the sn-2 position is commonly missing a fatty acid residue due to deacylation, leaving only the sn-3 hydroxyl esterified to a fatty acid. Moreover, a key enzyme in the production of LPA, autotaxin (lysoPLD/NPP2), may be the product of an oncogene, as many tumor types up-regulate autotoxin. The concentrations of LPA in human plasma and serum have been reported, including determinations made using sensitive and specific LC/MS procedures. For example, in freshly prepared human serum allowed to sit at 25° C. for one hour, LPA concentrations have been estimated to be approximately 1.2 mM, with the LPA analogs 16:0, 18:1, 18:2, and 20:4 being the predominant species. Similarly, in freshly prepared human plasma allowed to sit at 25° C. for one hour, LPA concentrations have been estimated to be approximately 0.7 mM, with 18:1 and 18:2 LPA being the predominant species.

LPA mediates its biological functions predominantly by binding to a class of multiple transmembrane G protein-coupled receptors (GPCR). Five LPA-specific GPCRs, termed LPA1-5, have been identified to date; they show both overlapping and distinct signaling properties and tissue expression. The LPA1-3 receptors belong to the so-called EDG subfamily (EGD2/LPA1, EDG4/LPA2, and EDG7/LPA3) of GPCRs with 50% sequence similarity to each other. Their closest relative is the cannabinoid CB1 receptor, which binds the bioactive lipids 2-arachidonoyl-glycerol (2-AG) and arachidonoyl-ethanolamine. Two newly identified LPA receptors, termed LPA4 (formerly GPR23/p2y9) and LPA5 (formerly GPR92) are more closely related to the P2Y nucleotide receptors. In addition, LPA recognizes the intracellular receptor, PPRgamma.

LPA1 is expressed in a wide range of tissues and organs whereas LPA2 and LPA3 show more restricted expression profile. However, LPA2 and LPA3 expressions were shown to be increased in ovarian and colon cancers and inflammation, suggesting that the main role of LPA2 and LPA3 is in pathophysiological conditions.

The role of these receptors has been in part elucidated by receptor knockout studies in mice. LPA1-deficient mice show partial postnatal lethality due to a suckling defect resulting from impaired olfaction. LPA1-deficient mice are also protected from lung fibrosis in response to bleomycin-induced lung injury. Furthermore, mice lacking the LPA1 receptor gene lose the nerve injury-induced neuropathic pain behaviors and phenomena.

In contrast, mice lacking LPA2 receptors appear to be normal. LPA3 receptor knockout mice have reduced litter size due to delayed blastocyst implantation and altered embryo spacing, and LPA3-deficient uteri show reduced cyclooxygenase-2 (COX-2) expression and prostaglandin synthesis; while exogenous administration of PGE2 into LPA3-deficient female mice has been reported to rescue the implantation defect.

LPAs influence a wide range of biological responses, including induction of cell proliferation, stimulation of cell migration and neurite retraction, gap junction closure, and even slime mold chemotaxis. The body of knowledge about the biology of LPA continues to grow as more and more cellular systems are tested for LPA responsiveness. The major physiological and pathophysiological effects of LPA include, for example:

Wound healing: it is now known that, in addition to stimulating cell growth and proliferation, LPA promote cellular tension and cell-surface fibronectin binding, which are important events in wound repair and regeneration.

Apoptosis: recently, anti-apoptotic activity has also been ascribed to LPA, and it has recently been reported that peroxisome proliferation receptor gamma is a receptor/target for LPA.

Blood vessel maturation: autotaxin, a secreted lysophospholipase D responsible for producing LPAs, is essential for blood vessel formation during development. In addition, unsaturated LPAs were identified as major contributors to the induction of vascular smooth muscle cell dedifferentiation.

Edema and vascular permeability: LPA induces plasma exudation and histamine release in mice.

Inflammation: LPA acts as inflammatory mediator in human corneal epithelial cells. LPA participates in corneal wound healing and stimulates the release of ROS in lens. LPA can also re-activate HSV-1 in rabbit cornea.

The bite of the venomous spider, Loxosceles reclusa (brown recluse spider), causes necrotic ulcers that can cause serious and long lasting tissue damage, and occasionally death. The pathology of wounds generated from the bite of this spider consists of an intense inflammatory response mediated by AA and prostaglandins. The major component of the L. reclusa spider venom is the phospholipase D enzyme often referred to as sphingomyelinase D (SMase D), which hydrolyzes sphingomyelin to produce C1P. It has been found, however, that lysophospholipids with a variety of headgroups are hydrolysed by the L. reclusa enzyme to release LPA. It is believed that anti-LPA agents will be useful in reducing or treating inflammation of various types, including but not limited to inflammation resulting from L. reclusa envenomation.

Fibrosis and scar formation: LPA inhibits TGF-mediated stimulation of type I collagen mRNA stability via an ERK-dependent pathway in dermal fibroblasts. Moreover, LPA have some direct fibrogenic effects by stimulating collagen gene expression and proliferation of fibroblasts.

Immune response: LPA, like S1P, has been shown to play a role in the immune response through modulation of immune-related cells. These lipids promote T-cell migration to sites of immune response and regulate proliferation of T cells as well as secretion of various cytokines.

Neurotrauma: as has recently been discovered and as will be shown in examples below, LPA levels and levels of LPA metabolites rise in TBI and thus LPA and these metabolites are biomarkers for neurotrauma. In addition, antibody neutralization of LPA has been shown to be neuroprotective in both TBI and SCI as shown in the examples below.

Thus, agents that reduce the effective concentration of LPA, such as Lpath's anti-LPA mAb, are believed to be useful in methods for treating diseases and conditions such as those associated with neurotrauma, wound healing and fibrosis, apoptosis, angiogenesis and neovascularization, vascular permeability and inflammation, that are associated with an undesired, excessive or aberrant level of LPA. Recently, the applicants have developed several monoclonal antibodies against LPAs. These anti-LPA antibodies neutralize various LPAs and mitigate their biologic and pharmacologic action. Anti-LPA antibodies are, therefore, believed to be useful in prevention and/or treatment of various diseases and conditions associated with excessive, unwanted or aberrant levels of LPA.

### C. Detection of LPA and Diagnosis/Detection of LPA-Associated Conditions

Methods for separating and semi-quantitatively measuring phospholipids such as LPA using techniques such as thin-layer chromatography (TLC) followed by gas chromatography (GC) and/or mass spectrometry (MS) are known. For example, lipids may be extracted from a test sample of bodily fluid or tissue. Alternatively, thin-layer chromatography may be used to separate various phospholipids. Phospholipids and lysophospholipids can then be visualized on plates, for example, using ultraviolet light. Alternatively, lysophospholipid concentrations can be identified by physical measurements such as NMR or HPLC following isolation from phospholipids or as part of the phospholipid, or mass spectrometry (MS) or LC-MS. LPA levels have also been determined in ascites from ovarian cancer patients using an assay that relies on LPA-specific effects on eukaryotic cells in culture. However, these procedures are time-consuming, expensive, variable and typically only semi-quantitative. Enzymatic methods for detecting lysophospholipids such as LPA in biological fluids, and for correlating and detecting conditions associated with altered levels of lysophospholipids, are also known, e.g., U.S. Pat. Nos. 6,255,063 and 6, 248,553, originally assigned to Atairgin Technologies, Inc. and now commonly owned with the instant invention. Detection and/or quantitation of LPA using methods herein provides the basis for sensitive and specific methods for detection of LPA by way of specific LPA-binding agents, and thus for detection and diagnosis of diseases and conditions associated with LPA, particularly with aberrant levels of LPA. In particular, antibody-based detection and/or quantitation of LPA in patient samples provides the basis for a method of detection and/or diagnosing neurotrauma in a patient suspected of having sustained a neurotrauma, such as TBI, SCI, stroke or other damage to the CNS. Such detection methods and kits are particularly suited to rapid point-of-care testing and diagnosis. In one embodiment, lateral flow diagnostic methods and kits are used to quickly detect and quantitate LPA and/or LPA metabolite(s) in blood, urine or other patient samples.

### D. Theranostics and Companion Diagnostics

The concept of theranostics or companion diagnostics is growing, particularly in the field of personalized medicine. Effective companion diagnostics are believed to enhance clinical efficacy in two ways: first, by allowing responsive patients to be identified before treatment, and second, by enabling the efficacy of a treatment to be monitored in real time. In the first context, a companion diagnostic allows a practitioner to determine whether a given treatment will be effective on a particular patient, and possibly even at what dose, before the patient is treated. Put another way, companion diagnostics identify the patients who are most likely to benefit from a given regimen. Such products are increasingly used in treatment of certain cancers and other conditions with a defined genetic component. For example, Herceptin™ (Genentech) is an antibody-based treatment for breast cancer. A companion immunohistochemistry assay (HercepTest™) was developed to identify patients with HER2-positive metastatic breast cancer, since these patients respond better to Herceptin™ treatment. As described herein, the diagnostic methods and kits for rapid diagnosis of neurotrauma by measurement of LPA in bodily samples allows immediate identification of patients with neurotrauma, who are thus believed to benefit from treatment using anti-LPA antibodies and/or other drugs, or surgical and/or other procedures for treatment of neurotrauma. In the second context, the companion diagnostic allows a patient's response to treatment to be monitored in real time, for example through quantitation of LPA levels in patient samples following the start of therapeutic treatment after neurotrauma. For example, the efficacy of treatment can be determined and followed through measurements of LPA levels, particularly in CSF. Treatment may be, e.g., by administration of an anti-LPA antibody or LPA-binding antibody fragment. Anti-LPA antibodies have been shown to be effective in treating neurotrauma, e.g., spinal cord injury or traumatic brain injury, and in reducing damage that results from such trauma. The kits and methods herein are therefore believed to be useful as companion diagnostics to therapeutic agents for treatment of neurotrauma, particularly anti-LPA antibodies for treatment of TBI and other neurotrauma.

### E. Definitions

Several terms used in the context of the present invention are defined below. In addition to these terms, others are defined elsewhere in the specification, as necessary. Unless otherwise expressly defined herein, terms of art used in this specification will have their art-recognized meanings.

The term “aberrant” means excessive or unwanted, for example in reference to levels or effective concentrations of a cellular target such as a protein or bioactive lipid.

The term “antibody” (“Ab”) or “immunoglobulin” (Ig) refers to any form of a peptide, polypeptide derived from, modeled after or encoded by, an immunoglobulin gene, or fragment thereof, that is capable of binding an antigen or epitope. See, e.g., Immunobiology, Fifth Edition, C. A. Janeway, P. Travers, M., Walport, M. J. Shlomchiked., ed. Garland Publishing (2001). The term “antibody” is used herein in the broadest sense, and encompasses monoclonal, polyclonal or multispecific antibodies, minibodies, heteroconjugates, diabodies, triabodies, chimeric, antibodies, synthetic antibodies, antibody fragments, and binding agents that employ the complementarity determining regions (CDRs) (or variants thereof that retain antigen binding activity) of the parent antibody. Antibodies are defined herein as retaining at least one desired activity of the parent antibody. Desired activities can include the ability to bind the antigen specifically, the ability to inhibit proleration in vitro, the ability to inhibit angiogenesis in vivo, and the ability to alter cytokine profile(s) in vitro. Herein, antibodies and antibody fragments, variants, and derivatives may also be referred to as “immune-derived moieties”, in that such molecules, or at least the antigen-binding portion(s) thereof, have been derived from an anti-LPA antibody.

Native antibodies (native immunoglobulins) are usually heterotetrameric glycoproteins of about 150,000 Daltons, typically composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH), also referred to as the variable domain, followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues form an interface between the light- and heavy-chain variable domains. The terms “variable domain” and “variable region” are used interchangeably. The terms “constant domain” and “constant region” are also interchangeable with each other.

Three hypervariable regions (also known as complementarity determining regions or CDRs) in each of the VH and VL regions form the unique antigen binding site of the molecule. Most of the amino acid sequence variation in the antibody molecule is within the CDRs, giving the antibody its specificity for its antigen.

The light chains of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An “antibody derivative” is an immune-derived moiety, i.e., a molecule that is derived from an antibody. This comprehends, for example, antibody variants, antibody fragments, chimeric antibodies, humanized antibodies, multivalent antibodies, antibody conjugates and the like, which retain a desired level of binding activity for antigen.

As used herein, “antibody fragment” refers to a portion of an intact antibody that includes the antigen binding site or variable domains of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen-binding and include Fab, Fab′, F(ab′)2, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the VH and VL domains of antibody, wherein these domains are present in a single polypeptide chain. Generally, the Fv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds. Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

An “antibody variant,” in this case an anti-LPA antibody variant, refers herein to a molecule which differs in amino acid sequence from a native anti-LPA antibody amino acid sequence by virtue of addition, deletion and/or substitution of one or more amino acid residue(s) in the antibody sequence and which retains at least one desired activity of the parent anti-binding antibody. Desired activities can include the ability to bind the antigen specifically, the ability to inhibit proliferation in vitro, the ability to inhibit angiogenesis in vivo, and the ability to alter cytokine profile in vitro. The amino acid change(s) in an antibody variant may be within a variable domain or a constant region of a light chain and/or a heavy chain, including in the Fc region, the Fab region, the CH1 domain, the CH2 domain, the CH3 domain, and the hinge region. In one embodiment, the variant comprises one or more amino acid substitution(s) in one or more hypervariable region(s) of the parent antibody. For example, the variant may comprise at least one, e.g., from about one to about ten, and preferably from about two to about five, substitutions in one or more hypervariable regions of the parent antibody. Ordinarily, the variant will have an amino acid sequence having at least 65% amino acid sequence identity with the parent antibody heavy or light chain variable domain sequences, more preferably at least 75%, more preferably at 80%, more preferably at least 85%, more preferably at least 90%, and most preferably at least 95%. In some situations a sequence identity of at least 50% is preferred, where other characteristics of the molecule convey desired attributes such as binding and specificity. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind LPA and preferably has desired activities which are superior to those of the parent antibody. For example, the variant may have a stronger binding affinity, enhanced ability to reduce angiogenesis and/or halt tumor progression. To analyze such desired properties (for example les immunogenic, longer half-life, enhanced stability, enhanced potency), one should compare a Fab form of the variant to a Fab form of the parent antibody or a full length form of the variant to a full length form of the parent antibody, for example, since it has been found that the format of the anti-sphingolipid antibody impacts its activity in the biological activity assays disclosed herein. The variant antibody of particular interest herein can be one which displays at least about 10 fold, preferably at least about % 5, 25, 59, or more of at least one desired activity. The preferred variant is one that has superior biophysical properties as measured in vitro or superior activities biological as measured in vitro or in vivo when compared to the parent antibody.

An “anti-LPA agent” refers to any therapeutic agent that binds LPA, and includes antibodies, antibody variants, antibody-derived molecules or non-antibody-derived moieties that bind LPA and its variants.

A “bioactive lipid” refers to a lipid signaling molecule. Bioactive lipids are distinguished from structural lipids (e.g., membrane-bound phospholipids) in that they mediate extracellular and/or intracellular signaling and thus are involved in controlling the function of many types of cells by modulating differentiation, migration, proliferation, secretion, survival, and other processes. In vivo, bioactive lipids can be found in extracellular fluids, where they can be complexed with other molecules, for example serum proteins such as albumin and lipoproteins, or in “free” form, i.e., not complexed with another molecule species. As extracellular mediators, some bioactive lipids alter cell signaling by activating membrane-bound ion channels or GPCRs or enzymes or factors that, in turn, activate complex signaling systems that result in changes in cell function or survival. As intracellular mediators, bioactive lipids can exert their actions by directly interacting with intracellular components such as enzymes, ion channels, or structural elements such as actin.

Examples of bioactive lipids include sphingolipids such as ceramide, ceramide-1-phosphate (C1P), sphingosine, sphinganine, sphingosylphosphorylcholine (SPC) and sphingosine-1-phosphate (S1P). Sphingolipids and their derivatives and metabolites are characterized by a sphingoid backbone (derived from sphingomyelin). Sphingolipids and their derivatives and metabolites represent a group of extracellular and intracellular signaling molecules with pleiotropic effects on important cellular processes. They include sulfatides, gangliosides and cerebrosides. Other bioactive lipids are characterized by a glycerol-based backbone; for example, lysophospholipids such as lysophosphatidyl choline (LPC) and various lysophosphatidic acids (LPA), as well as phosphatidylinositol (PI), phosphatidylethanolamine (PEA), phosphatidic acid, platelet activating factor (PAF), cardiolipin, phosphatidylglycerol (PG) and diacylglyceride (DG). Yet other bioactive lipids are derived from arachidonic acid; these include the eicosanoids (including the eicosanoid metabolites such as the HETEs, cannabinoids, leukotrienes, prostaglandins, lipoxins, epoxyeicosatrienoic acids, and isoeicosanoids), non-eicosanoid cannabinoid mediators.

It may be preferable to target glycerol-based bioactive lipids (those having a glycerol-derived backbone, such as the LPAs) for antibody production, as opposed to sphingosine-based bioactive lipids (those having a sphingoid backbone, such as sphingosine and S1P). In other embodiments it may be desired to target arachidonic acid-derived bioactive lipids for antibody generation, and in other embodiments arachidonic acid-derived and glycerol-derived bioactive lipids but not sphingoid-derived bioactive lipids are preferred. Together the arachidonic acid-derived and glycerol-derived bioactive lipids may be referred to herein as “non-sphingoid bioactive lipids.”

Specifically excluded from the class of bioactive lipids as defined herein are phosphatidylcholine and phosphatidylserine, as well as their metabolites and derivatives that function primarily as structural members of the inner and/or outer leaflet of cellular membranes.

The term “biologically active,” in the context of an antibody or antibody fragment or variant, refers to an antibody or antibody fragment or antibody variant that is capable of binding the desired epitope and in some ways exerting a biologic effect. Biological effects include, but are not limited to, the modulation of a growth signal, the modulation of an anti-apoptotic signal, the modulation of an apoptotic signal, the modulation of the effector function cascade, and modulation of other ligand interactions.

A “biomarker” is a specific biochemical in the body which has a particular molecular feature that makes it useful for measuring the progress of disease or the effects of treatment.

“Cardiovascular therapy” encompasses cardiac therapy (treatment of myocardial ischemia and heart failure) as well as the prevention and/or treatment of other diseases associated with the cardiovascular system, such as heart disease. The term “heart disease” encompasses any type of disease, disorder, trauma or surgical treatment that involves the heart or myocardial tissue. Of particular interest are conditions associated with tissue remodeling. The term “cardiotherapeutic agent” refers to an agent that is therapeutic to diseases and diseases caused by or associated with cardiac and myocardial diseases and disorders.

A “carrier” refers to a moiety adapted for conjugation to a hapten, thereby rendering the hapten immunogenic. A representative, non-limiting class of carriers is proteins, examples of which include albumin, keyhole limpet hemocyanin, hemaglutanin, tetanus, and diptheria toxoid. Other classes and examples of suitable carriers are known in the art. These, as well as later discovered or invented naturally occurring or synthetic carriers, can be adapted for application in accordance with the disclosures herein.

As used herein, the expressions “cell,” “cell line,” and “cell culture” are used interchangeably and all such designations include progeny. Thus, the words “transformants” and “transformed cells” include the primary subject cell and cultures derived there from without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “chimeric” antibody (or immunoglobulin) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly, et al., infra; Morrison et al., Proc. Natl. Acad. Sci. U.S.A., vol. 81:6851 (1984)). One example of a chimeric antibody is an antibody containing murine variable domains (VL and VH) and human constant domains. However, antibody sequences may be vertebrate or invertebrate in origin, e.g., from mammal, bird or fish, including cartilaginous fish, rodents, canines, felines, ungulate animals and primates, including humans.

The term “combination therapy” refers to a therapeutic regimen that involves the provision of at least two distinct therapies to achieve an indicated therapeutic effect. For example, a combination therapy may involve the administration of two or more chemically distinct active ingredients, for example, a fast-acting chemotherapeutic agent and an anti-lipid antibody. Alternatively, a combination therapy may involve the administration of an anti-lipid antibody and/or one or more chemotherapeutic agents, alone or together with the delivery of another treatment, such as radiation therapy and/or surgery. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more anti-lipid antibody species, for example, an anti-LPA antibody, alone or in conjunction with one or more chemotherapeutic agents are combined with, for example, radiation and/or surgery, the drug(s) may be delivered before or after surgery or radiation treatment.

“Companion diagnostic” refers to a diagnostic test that is linked to a particular drug treatment or therapy. In particular, the diagnostic methods and kits for rapid diagnosis of neurotrauma by measurement of LPA or LPA metabolite(s) in bodily samples allows prompt identification of patients with neurotrauma, who are thus believed to benefit from treatment using anti-LPA antibodies or LPA-binding antibody fragments and/or other drugs, or surgical and/or other procedures for treatment of neurotrauma.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “derivatized bioactive lipid” is a bioactive lipid, e.g., LPA, which has a polar head group and at least one hydrocarbon chain, wherein a carbon atom within the hydrocarbon chain is derivatized with a pendant reactive group [e.g., a sulfhydryl (thiol) group, a carboxylic acid group, a cyano group, an ester, a hydroxy group, an alkene, an alkyne, an acid chloride group or a halogen atom] that may or may not be protected. This derivatization serves to activate the bioactive lipid for reaction with a molecule, e.g., for conjugation to a carrier.

A “derivatized bioactive lipid conjugate” refers to a derivatized bioactive lipid that is covalently conjugated to a carrier. The carrier may be a protein molecule or may be a moiety such as polyethylene glycol, colloidal gold, adjuvants or silicone beads. A derivatized bioactive lipid conjugate may be used as an immunogen for generating an antibody response, and the same or a different bioactive lipid conjugate may be used as a detection reagent for detecting the antibody thus produced. In some embodiments the derivatized bioactive lipid conjugate is attached to a solid support when used for detection.

To “detect” means to discover or ascertain the existence or presence of (e.g., a disease or condition).

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

“Diagnosis” means identification of an illness or other condition by examination of its symptoms, including test results and other measurements.

“Effective concentration” refers to the absolute, relative, and/or available concentration and/or activity, for example of certain undesired bioactive lipids. In other words, the effective concentration of a bioactive lipid is the amount of lipid available, and able, to perform its biological function. An immune-derived moiety such as, for example, a monoclonal antibody directed to a bioactive lipid (such as, for example, C1P) is able to reduce the effective concentration of the lipid by binding to the lipid and rendering it unable to perform its biological function. In this example, the lipid itself is still present (it is not degraded by the antibody, in other words) but can no longer bind its receptor or other targets to cause a downstream effect, so “effective concentration” rather than absolute concentration is the appropriate measurement. Methods and assays exist for directly and/or indirectly measuring effective concentrations of bioactive lipids.

An “epitope” or “antigenic determinant” refers to that portion of an antigen that reacts with an antibody antigen-binding portion derived from an antibody.

The term “expression cassette” refers to a nucleotide molecule capable of affecting expression of a structural gene (i.e., a protein coding sequence, such as an antibody chain) in a host compatible with such sequences. Expression cassettes include at least a promoter operably linked with the polypeptide-coding sequence, and, optionally, with other sequences, e.g., transcription termination signals. Additional regulatory elements necessary or helpful in effecting expression may also be used, e.g., enhancers. Thus, expression cassettes include plasmids, expression vectors, recombinant viruses, any form of recombinant “naked DNA” vector, and the like.

A “fully human antibody” can refer to an antibody produced in a genetically engineered (i.e., transgenic) animal, typically a mammal, usually a mouse (e.g., as can be obtained from Medarex) that, when presented with a suitable immunogen, can produce a human antibody that does not necessarily require CDR grafting. These antibodies are fully “human” in that they generated from from an animal (e.g., a transgenic mouse) in which the non-human antibody genes are replaced or suppressed and replaced with some or all of the human immunoglobulin genes. In other words, antibodies include those generated against bioactive lipids, specifically LPA, when presented in an immunogenic form to mice or other animals genetically engineered to produce human frameworks for relevant CDRs.

A “hapten” is a substance that is non-immunogenic but can react with an antibody or antigen-binding portion derived from an antibody. In other words, haptens have the property of antigenicity but not immunogenicity. A hapten is generally a small molecule that can, under most circumstances, elicit an immune response (i.e., act as an antigen) only when attached to a carrier, for example, a protein, polyethylene glycol (PEG), colloidal gold, silicone beads, or the like. The carrier may be one that also does not elicit an immune response by itself.

The term “heteroconjugate antibody” can refer to two covalently joined antibodies. Such antibodies can be prepared using known methods in synthetic protein chemistry, including using crosslinking agents. As used herein, the term “conjugate” refers to molecules formed by the covalent attachment of one or more antibody fragment(s) or binding moieties to one or more polymer molecule(s).

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g., murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. In some instances, framework region (FR) residues of the human immunoglobulin are replaced by corresponding non-human residues.

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin. See, e.g., Cabilly, et al., U.S. Pat. No. 4,816,567; Cabilly, et al., European Patent No. 0,125,023 B1; Boss, et al., U.S. Pat. No. 4,816,397; Boss, et al., European Patent No. 0,120,694 B1; Neuberger, et al., WO 86/01533; Neuberger, et al., European Patent No. 0,194,276 B1; Winter, U.S. Pat. No. 5,225,539; Winter, European Patent No. 0,239,400 B1; Padlan, et al., European Patent Application No. 0,519,596 A1; Queen, et al. (1989), Proc. Nat'l Acad. Sci. USA, vol. 86:10029-10033). For further details, see Jones et al., Nature 321:522-525 (1986); Reichmann et al., Nature 332:323-329 (1988); and Presta, Curr. Op. Struct. Biol. 2:593-596 (1992) and Hansen, WO2006105062.

The term “hyperproliferative disorder” refers to diseases and disorders associated with, the uncontrolled proliferation of cells, including but not limited to uncontrolled growth of organ and tissue cells resulting in cancers and benign tumors. Hyperproliferative disorders associated with endothelial cells can result in diseases of angiogenesis such as angiomas, endometriosis, obesity, age-related macular degeneration and various retinopathies, as well as the proliferation of endothelial cells and smooth muscle cells that cause restenosis as a consequence of stenting in the treatment of atherosclerosis. Hyperproliferative disorders involving fibroblasts (i.e., fibrogenesis) include, without limitation, disorders of excessive scarring (i.e., fibrosis) such as age-related macular degeneration, cardiac remodeling and failure associated with myocardial infarction, as well asexcessive wound healing such as commonly occurs as a consequence of surgery or injury, keloids, and fibroid tumors and stenting.

An “immunogen” is a molecule capable of inducing a specific immune response, particularly an antibody response in an animal to whom the immunogen has been administered. The immunogen may be a derivatized bioactive lipid conjugated to a carrier, i.e., a “derivatized bioactive lipid conjugate”. The derivatized bioactive lipid conjugate used as the immunogen may be used as capture material for detection of the antibody generated in response to the immunogen. Thus the immunogen may also be used as a detection reagent. Alternatively, the derivatized bioactive lipid conjugate used as capture material may have a different linker and/or carrier moiety from that in the immunogen.

To “inhibit,” particularly in the context of a biological phenomenon, means to decrease, suppress or delay. For example, a treatment yielding “inhibition of tumorigenesis” may mean that tumors do not form at all, or that they form more slowly, or are fewer in number than in the untreated control.

An “isolated” composition is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the composition is an antibody and will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition, such as one that is conjugated directly or indirectly to the antibody. The label may itself be detectable by itself (e.g., radioisotope labels or fluorescent labels) or, in the case of an enzymatic label, may catalyze chemical alteration of a substrate compound or composition that is detectable.

A “liposome” is a small vesicle composed of various types of lipids, phospholipids and/or surfactant that is useful for delivery of a drug (such as the anti-sphingolipid antibodies disclosed herein and, optionally, a chemotherapeutic agent) to a mammal. The components of the liposome are commonly arranged in a bilayer formation, similar to the lipid arrangement of biological membranes. An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of non-engineered cells.

In the context of this disclosure, a “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, solids include dried compositions prepared by lyophilization, freeze-drying, precipitation, and similar procedures.

The expression “linear antibodies” when used throughout this application refers to the antibodies described in Zapata et al. Protein Eng. 8(10):1057-1062 (1995). Briefly, these antibodies comprise a pair of tandem Fd segments (VH-CH1-VH-CH1) that form a pair of antigen binding regions. Linear antibodies can be bispecific or monospecific.

The term “LPA metabolites” refers to compounds from which LPAs are made, as well as those that result from the degradation of LPAs; that is, compounds that are involved in the lysophospholipid metabolic pathways. The term “metabolic precursors” may also be used to refer to compounds from which LPAs are made. Examples of LPA metabolites include lyso-platelet activating factor (lyso-PAF), LPC, and lysophosphatidylglycerol (LPG), all of which can be converted to LPA [Gesta, S et al., (2002) J. Lipid Res. 43: 904-910]; as well as monoacylglycerol (MAG), into which LPA can be converted by lipid phosphatases. Mills and Moolenaar (2003) Nature Reviews Cancer 3, 582-591.

The term “monoclonal antibody” (mAb) as used herein refers to an antibody obtained from a population of substantially homogeneous antibodies, or to said population of antibodies. The individual antibodies comprising the population are essentially identical, except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Monotherapy” refers to a treatment regimen based on the delivery of one therapeutically effective compound, whether administered as a single dose or several doses over time.

The term “multispecific antibody” can refer to an antibody, or a monoclonal antibody, having binding properties for at least two different epitopes. In one embodiment, the epitopes are from the same antigen. In another embodiment, the epitopes are from two or more different antigens. Methods for making multispecific antibodies are known in the art. Multispecific antibodies include bispecific antibodies (having binding properties for two epitopes), trispecific antibodies (three epitopes) and so on. For example, multispecific antibodies can be produced recombinantly using the co-expression of two or more immunoglobulin heavy chain/light chain pairs. Alternatively, multispecific antibodies can be prepared using chemical linkage. One of skill can produce multispecific antibodies using these or other methods as may be known in the art. Multispecific antibodies include multispecific antibody fragments. One example of a multispecific (in this case, bispecific) antibody is an antibody having binding properties for an S1P epitope and a C1P epitope, which thus is able to recognize and bind to both S1P and C1P. Another example of of a bispecific antibody is an antibody having binding properties for an epitope from a bioactive lipid and an epitope from a cell surface antigen. Thus the antibody is able to recognize and bind the bioactive lipid and is able to recognize and bind to cells, e.g., for targeting purposes.

“Neoplasia” or “cancer” refers to abnormal and uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

“Neurotrauma” refers to injury or damage to the central nervous system (CNS), i.e., the brain and/or spinal cord. Neurotrauma includes traumatic brain injury (TBI), spinal cord injury (SCI), and stroke (hemorrhagic and ischemic).

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The “parent” antibody herein is one that is encoded by an amino acid sequence used for the preparation of the variant. The parent antibody may be a native antibody or may already be a variant, e.g., a chimeric antibody. For example, the parent antibody may be a humanized or human antibody.

A “patentable” composition, process, machine, or article of manufacture means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

The term “pharmaceutically acceptable salt” refers to a salt, such as used in formulation, which retains the biological effectiveness and properties of the agents and compounds described herein and which are is biologically or otherwise desirable. In many cases, the agents and compounds described herein are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts (see Berge, et al. (1977) J. Pharm. Sci., vol. 66, 1-19).

A “plurality” means more than one.

“Point-of-care testing” means medical testing or diagnosis at or near the site of patient care.

The term “promoter” includes all sequences capable of driving transcription of a coding sequence in a cell. Thus, promoters used in the constructs described herein include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. Transcriptional regulatory regions suitable for use include but are not limited to the human cytomegalovirus (CMV) immediate-early enhancer/promoter, the SV40 early enhancer/promoter, the E. coli lac or trp promoters, and other promoters known to control expression of genes in prokaryotic or eukaryotic cells or their viruses.

The term “recombinant DNA” refers to nucleic acids and gene products expressed therefrom that have been engineered, created, or modified by man. “Recombinant” polypeptides or proteins are polypeptides or proteins produced by recombinant DNA techniques, for example, from cells transformed by an exogenous DNA construct encoding the desired polypeptide or protein. “Synthetic” polypeptides or proteins are those prepared by chemical synthesis.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

By “solid phase” is meant a non-aqueous matrix such as one to which an antibody can adhere directly or indirectly. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes beads or a discontinuous solid phase of discrete particles, such as those described in U.S. Pat. No. 4,275,149.

The term “species” is used herein in various contexts, e.g., a particular species of chemotherapeutic agent. In each context, the term refers to a population of chemically indistinct molecules of the sort referred in the particular context.

The term “specific” or “specificity” in the context of antibody-antigen interactions refers to the selective, non-random interaction between an antibody and its target epitope. Here, the term “antigen” refers to a molecule that is recognized and bound by an antibody molecule or other immune-derived moiety. The specific portion of an antigen that is bound by an antibody is termed the “epitope”. This interaction depends on the presence of structural, hydrophobic/hydrophilic, and/or electrostatic features that allow appropriate chemical or molecular interactions between the molecules. Thus an antibody is commonly said to “bind” (or “specifically bind”) or be “reactive with” (or “specifically reactive with), or, equivalently, “reactive against” (or “specifically reactive against”) the epitope of its target antigen. Antibodies are commonly described in the art as being “against” or “to” their antigens as shorthand for antibody binding to the antigen. Thus an “antibody that binds C1P,” an “antibody reactive against C1P,” an “antibody reactive with C1P,” an “antibody to C1P” and an “anti-C1P antibody” all have the same meaning in the art. Antibody molecules can be tested for specificity of binding by comparing binding to the desired antigen to binding to unrelated antigen or analogue antigen or antigen mixture under a given set of conditions. Preferably, an antibody will lack significant binding to unrelated antigens, or even analogs of the target antigen.

Herein, “stable” refers to an interaction between two molecules (e.g., a peptide and a TLR molecule) that is sufficiently stable such that the molecules can be maintained for the desired purpose or manipulation. For example, a “stable” interaction between a peptide and a TLR molecule refers to one wherein the peptide becomes and remains associated with a TLR molecule for a period sufficient to achieve the desired effect.

A “subject” or “patient” refers to an animal to which treatment is given. Animals that can be treated include vertebrates, with mammals such as bovine, canine, equine, feline, ovine, porcine, and primate (including humans and non-human primates) animals being particularly preferred examples.

A “surrogate marker” refers to laboratory measurement of biological activity within the body that indirectly indicates the effect of treatment on disease state. Examples of surrogate markers for hyperproliferative and/or cardiovascular conditions include SPHK and/or S1PRs.

A “therapeutic agent” refers to a drug or compound that is intended to provide a therapeutic effect including, but not limited to: anti-inflammatory drugs including COX inhibitors and other NSAIDS, anti-angiogenic drugs, chemotherapeutic drugs as defined above, cardiovascular agents, immunomodulatory agents, agents that are used to treat neurodegenerative disorders, opthalmic drugs, etc.

A “therapeutically effective amount” (or “effective amount”) refers to an amount of an active ingredient, e.g., an agent such as an antibody, sufficient to effect treatment when administered to a subject in need of such treatment. Accordingly, what constitutes a therapeutically effective amount of a composition may be readily determined by one of ordinary skill in the art. The therapeutically effective amount will depend upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient).

The compositions described herein are used in methods of bioactive lipid-based therapy. As used herein, the terms “therapy” and “therapeutic” encompasses the full spectrum of prevention and/or treatments for a disease, disorder or physical trauma. A “therapeutic” agent may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of at least one symptom of a disease or disorder being treated; or may act to minimize the time required, the occurrence or extent of any discomfort or pain, or physical limitations associated with recuperation from a disease, disorder or physical trauma; or may be used as an adjuvant to other therapies and treatments.

The term “treatment” or “treating” means any treatment of a disease or disorder, including preventing or protecting against the disease or disorder (that is, causing the clinical symptoms not to develop); inhibiting the disease or disorder (i.e., arresting, delaying or suppressing the development of clinical symptoms; and/or relieving the disease or disorder (i.e., causing the regression of clinical symptoms). As will be appreciated, it is not always possible to distinguish between “preventing” and “suppressing” a disease or disorder because the ultimate inductive event or events may be unknown or latent. Those “in need of treatment” include those already with the disorder as well as those in which the disorder is to be prevented. Accordingly, the term “prophylaxis” will be understood to constitute a type of “treatment” that encompasses both “preventing” and “suppressing”. The term “protection” thus includes “prophylaxis”.

The term “therapeutic regimen” means any treatment of a disease or disorder using chemotherapeutic and cytotoxic agents, radiation therapy, surgery, gene therapy, DNA vaccines and therapy, siRNA therapy, anti-angiogenic therapy, immunotherapy, bone marrow transplants, aptamers and other biologics such as antibodies and antibody variants, receptor decoys and other protein-based therapeutics.

The “variable” region of an antibody comprises framework and complementarity determining regions (CDRs, otherwise known as hypervariable regions). The variability is not evenly distributed throughout the variable domains of antibodies. It is concentrated in six CDR segments, three in each of the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the beta-sheet structure. The term “hypervariable region” when used herein refers to the amino acid residues of an antibody which are responsible for antigen binding. The hypervariable region comprises amino acid residues from a “complementarity determining region” or “CDR” (for example residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (for example residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

A “vector” or “plasmid” or “expression vector” refers to a nucleic acid that can be maintained transiently or stably in a cell to effect expression of one or more recombinant genes. A vector can comprise nucleic acid, alone or complexed with other compounds. A vector optionally comprises viral or bacterial nucleic acids and/or proteins, and/or membranes. Vectors include, but are not limited, to replicons (e.g., RNA replicons, bacteriophages) to which fragments of DNA may be attached and become replicated. Thus, vectors include, but are not limited to, RNA, autonomous self-replicating circular or linear DNA or RNA and include both the expression and non-expression plasmids. Plasmids can be commercially available, publicly available on an unrestricted basis, or can be constructed from available plasmids as reported with published protocols. In addition, the expression vectors may also contain a gene to provide a phenotypic trait for selection of transformed host cells such as dihydrofolate reductase or neomycin resistance for eukaryotic cell culture, or such as tetracycline or ampicillin resistance in E. coli.

### SUMMARY OF THE INVENTION

The instant application provides methods of detecting or diagnosing neurotrauma in a subject comprising determining LPA levels and/or LPA metabolite levels in a biological sample from the subject, wherein elevated LPA levels and/or LPA metabolite levels in the sample (compared to typical values for uninjured controls) are indicative of neurotrauma. Neurotrauma includes traumatic brain injury, spinal cord injury, or stroke. LPA levels may refer to total LPA levels or to levels of one or more LPA isoforms, such as one or more of 16:0, 18:0, 18:1, 18:2, or 20:4 acyl LPAs. The biological sample may be a tissue sample or a bodily fluid sample, e.g., a sample of cerebrospinal fluid (CSF), blood, plasma, urine, or central nervous system tissue. The LPA metabolite may be, for example, lysophosphatidylcholine (LPC) or lyso-platelet activating factor (lyso-PAF).

The determining of LPA or LPA metabolite levels may be performed by any suitable now-known or later developed method, for example, a method based on an LPA-binding agent, such as an antibody-based method, e.g., enzyme-linked immunosorbent assay (ELISA), lateral flow immunoassay (LFIA) or immunohistochemistry (IHC); a physical measurement method, e.g., mass spectrometry or liquid chromatography/mass spectrometry; or an enzymatic method. The antibody used in the antibody-based method is an antibody that specifically binds LPA, or an antigen-binding fragment thereof. The method may further comprise determining levels of at least one additional biomarker for neurotrauma in the biological sample, such as a lipid or protein biomarker. Examples of additional protein or lipid biomarkers are ubiquitin C-terminal hydrolase (UCH-L1), glial fibrillary acidic protein (GFAP), the phosphorylated form of the high-molecular-weight neurofilament subunit NF-H (pNF-H), an LPA metabolite and 12-hydroxyeicosatetraenoic acid (12-HETE). In one embodiment, the method of detecting or diagnosing neurotrauma in a subject comprises determining LPA levels in a biological sample from said subject, wherein the determining of LPA levels is by an antibody-based method using an antibody that is specifically reactive with LPA, or antigen-binding fragment thereof. The method may further comprise use of a derivatized, e.g., thiolated, LPA bound directly or indirectly to a solid support or a carrier moiety, e.g., polyethylene glycol, colloidal gold, adjuvant, a silicone bead, colored particle, or a protein. In other representative embodiments, LPA is the first biomarker and the additional biomarker may be an LPA metabolite or 12-HETE; LPC is the first biomarker and the additional biomarker may be LPA, lyso-PAF, or 12-HETE; or the first biomarker is lyso-PAF and the additional biomarker may be LPA, LPC or 12-HETE.

Further comprehended by the invention are kits for detecting or diagnosing neurotrauma in a subject, Such kits typically include components for determining LPA and/or LPA metabolite levels in a biological sample from a subject, wherein elevated levels compared to typical uninjured controls are indicative of neurotrauma. The components for determining LPA or LPA metabolite levels may be, e.g., an antibody-based method or an enzymatic method, and the biological sample may be a tissue sample or a bodily fluid sample, e.g., a sample of cerebrospinal fluid (CSF), blood, plasma, urine or central nervous system tissue. In some embodiments, the kit components for detection of LPA or LPA metabolite levels is by way of a lateral flow immunoassay comprising an antibody, or antigen-binding fragment thereof, which specifically binds to the LPA or the LPA metabolite. The lateral flow immunoassay may further comprise a derivatized LPA or LPA metabolite that is directly or indirectly bound to a solid support or a carrier moiety, e.g., polyethylene glycol, colloidal gold, adjuvant, a silicone bead, a latex bead, or a protein e.g., keyhole limpet hemocyanin, albumin, bovine thyroglobulin, or other carriers and supports known in the art. In some embodiments, the carrier is colored or carries a detectable label. In some embodiments, more than one biomarker (e.g., LPA and an LPA metabolite, LPA and 12-HETE, or an LPA metabolite and a protein biomarker) is detected in the same lateral flow assay. In some embodiments, the component(s) for determining LPA or LPA metabolite levels is an ELISA assay and the kit comprises an antibody, or antigen-binding fragment thereof, which specifically binds to the LPA or LPA metabolite. In some embodiments, more than one biomarker (e.g., LPA and an LPA metabolite, LPA and 12-HETE, or an LPA metabolite and a protein biomarker) is detected in the same ELISA. The kit may further comprise a derivatized LPA or LPA metabolite that is directly or indirectly bound to a solid support or a carrier moiety, e.g., polyethylene glycol, colloidal gold, adjuvant, a silicone bead, latex bead, other colored particle or a protein e.g., keyhole limpet hemocyanin, albumin, bovine thyroglobulin, or soybean trypsin inhibitor. In some embodiments, the carrier is colored or carries a detectable label. The carrier moiety may be attached to a solid support. The kit may further comprise a means for determining levels of at least one additional biomarker for neurotrauma in the biological sample, as described supra.

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

A brief summary of each of the figures and tables described in this specification are provided below. This application contains at least one figure executed in color. Copies of this application with color drawings will be provided upon request and payment of the necessary fee.

FIGS. 1a-1c are an organic synthesis scheme for making of a typical thiolated-S1P analog that was used as a key component of an immunogen, as well as a key component of the laydown material for the ELISA and BiaCore assays.

FIGS. 2a-2b are an organic synthesis scheme for making the thiolated-related fatty acid used in the synthesis of the thiolated-LPA analog of FIG. 3a.

FIGS. 3a-3b are an organic synthesis scheme for making the thiolated-LPA analog that is a key component of an immunogen, as well as a key component of the laydown material for the ELISA and other assays.

FIGS. 4a-4b are a two-part figure showing that treatment with anti-LPA antibody B3 improves functional recovery following SCI. mBBB score and grid walking test were measured up to 5 weeks post SCI. Treatment with B3 (n=7) compared to isotype control antibody (con; n=8), given for two weeks following SCI.Data are mean±SEM; *p<0.05. FIG. 4a is a line graph showing the mBBB open field locomotor test scores; FIG. 4b is a line graph showing grid walking test scores.

FIG. 5 is a bar graph showing that antibody to LPA improves neuronal survival following spinal cord injury (SCI). Quantitation of number of traced neuronal cells rostral to lesion site is significantly higher in antibody treated mice compared to controls. Data are mean±SEM;**p<0.001.

FIGS. 6a-6b are a two-part bar graph showing that anti-LPA mAb (B3) reduces glial scar following SCI. Immunostaining at the injury site of mice spinal cords, 2 weeks following SCI. Mice received or not anti LPA mAb (B3, 0.5 mg/mouse) subcutaneously twice a week for two weeks, starting just after SCI. B3 treatment reduces the amount of reactive astrocytes (GFAP and CSPG cells) (panel A) and increases the amount of neurons (NeuN) close to the lesion site (panel B).

FIGS. 7a-7b are a micrograph showing mouse brains after cortical injury. Panel A on the left shows a mouse brain with an area of hemorrhage as typically seen after TBI in the cortical impact model. Panel B on the right shows a mouse brain after TBI in the same model, but treated with anti-LPA antibody. The hemorrhage normally observed in this model is greatly reduced.

FIGS. 8a-8b are a two-part figure showing that anti-LPA antibody is protective in a mouse model of traumatic brain injury. FIG. 8a shows brains of 12 mice following TBI. The 6 brains in the top panel (Con) were from mice that received no antibody treatment prior to TBI. The 6 brains in the lower panel (Mab) were from mice that received the anti-LPA antibody B3, 0.5 mg/mouse i.v., prior to the application of a single impact injury (1.5 mm depth). Mice were taken down 24 hrs following injury. FIG. 8b shows histological quantitation of the infarct volumes in these animals. As shown, the decrease in infarct size in anti-LPA antibody-treated mice compared to controls is statistically significant.

FIG. 9 is a scatter plot showing that anti-LPA mAb intervention treatment significantly reduces neurotrauma following TBI. Mice were subjected to TBI using Controlled Cortical Impact (CCI) and treated with either control mAb or B3 given as single i.v. dose of 25 mg/kg 30 min after injury. Data were obtained 2 days after injury and infarct size for each animal was quantified histologically. Data are means±SEM, n=8 animals per group in from two independent, blinded studies.

FIGS. 10a-10d are a multi-part figure showing that anti-LPA mAb intervention treatment significantly reduces neurotrauma following TBI. Mice were subjected to TBI using Controlled Cortical Impact (CCI) and treated with either control mAb or B3 given as single i.v. dose of 25 mg/kg 30 min after injury. Data were obtained seven days after injury. FIGS. 10a and 10b is a pair of bar graphs showing histological quantification of infarct size by MRI, assessed on day 1 (top) and day 7 (bottom) post-injury. Data are means±SEM, n=8 animals per group in from two independent, blinded studies. *p<0.05. FIGS. 10c and 10d is two pairs of photographs showing representative MRI images of mouse brains following TBI and subsequent treatment with anti-LPA antibody B3 (right) or isotype control (left) antibody. The top pair of images was taken on day 1 and the bottom pair was taken on day 7 post-injury.

FIG. 11 is a line graph showing total LPA levels in the CSF of each of 5 TBI patients at 1, 2, 3, 4, and 5 days post-injury. Three uninjured, normal control subjects showed average LPA levels of 0.050±0.012 uM.

FIG. 12 is a bar graph showing average total LPA levels in the CSF of the same five TBI patients, measured 24 hours after injury, compared to average total LPA in CSF of controls.

FIG. 13 is a bar graph showing levels of several physiologically relevant LPA isoforms in CSF samples from neurotrauma patients vs controls.

FIGS. 14a-14b are a two-part figure showing scatter plots of LPA precursors lyso-PAF (FIG. 14a) and LPC (FIG. 14b) in cerebrospinal fluid (CSF) of traumatic brain injury patients at days 1, 2, 3, 4, and 5 after injury.

FIG. 15 is a line graph showing total LPA levels in the CSF of each of 8 neurotrauma (TBI) patients at 0, 1, 2, 3, 4, and 5 days post-injury. Three uninjured, normal control subjects showed average LPA levels of 0.13±0.09 uM.

FIG. 16 is a scatter plot showing a time course of total acyl LPA levels in CSF samples from 11 neurotrauma (TBI) patients from 0 to 144 hours post injury.

FIG. 17 is a bar graph showing total acyl LPA levels over time in CSF from 11 neurotrauma (TBI) patients in FIG. 16, along with controls. Patient samples are grouped by time of sampling (<14 hours post injury, <24 hours post injury, <36 hours post injury and >36 hours post injury).

FIG. 18 is a bar graph showing levels of individual acyl LPA isoforms (16:0 in red, 18:0 in blue, 18:1 in magenta, 18:2 in turquoise and 20:4 in green) in CSF from 13 neurotrauma (TBI) patients over time from day 0 to day 5 post-injury, along with controls.

FIGS. 19a-19c are a set of three line graphs showing correlation of LPA levels in the CSF to three standard measures of severity of injury. CSF was collected within 24 hr post injury from 8 neurotrauma (TBI) patients. FIG. 19a shows the correlation of LPA levels in CSF to Glasgow Coma Score (GCS); FIG. 19b shows correlation of LPA levels in the CSF to Extended Glasgow Outcome Scale (GOSE); FIG. 19c shows correlation of LPA levels in the CSF to Injury Severity Score (ISS).

### DETAILED DESCRIPTION OF THE INVENTION

The instant invention provides methods and kits for detection and diagnosis of neurotrauma, by measuring LPA and/or LPA metabolite levels in fluid or tissue samples from patients suspected of having sustained a CNS injury or damage. It has recently been shown (see examples below) that LPA levels and certain LPA metabolite levels rise significantly in cerebrospinal fluid (CSF) of human subjects following TBI, compared to normal control; in other words, LPA is a biomarker for neurotrauma. This observation will open doors to novel and highly useful diagnostic tests and procedures for rapid diagnosis of neurotrauma.

LPA and LPA metabolites can be measured using a variety of means, including enzymatic means, physical measurements (e.g., mass spectrometry, LC-MS), and methods which rely on specific LPA-binding agents such as antibodies to LPA, aptamers that bind LPA, LPA receptor fragments and the like. Derivatized LPA may also be useful in detection and measurement of LPA, such as in antibody-based methods such as ELISA or other immunochemical assays, and may be used in preparing labeled LPA (radiolabelled or otherwise).

Highly specific anti-LPA antibodies have been generated by Lpath, Inc. and have been demonstrated to have therapeutic utility against cancer, fibrosis and other conditions. More recently, anti-LPA antibodies have been shown to be neuroprotective in animal models of neurotrauma, e.g., TBI, stroke and SCI. Thus anti-LPA antibodies have both therapeutic and diagnostic uses related to neurotrauma, and may accordingly be used in companion diagnostics.

A. Derivatized and/or Conjugated LPA

1. Compositions

The present invention may utilize LPA which has been derivatized in such a way as to facilitate the immunogenic response (i.e., antibody production) and/or to allow conjugation of the LPA molecule to a carrier molecule or other moiety such as a label or solid support. In one embodiment, a carbon atom within the hydrocarbon chain of the LPA is derivatized with a pendant reactive group [e.g., a sulfhydryl (thiol) group, a carboxylic acid group, a cyano group, an ester, a hydroxy group, an alkene, an alkyne, an acid chloride group or a halogen atom]. This derivatization serves to activate the bioactive lipid for reaction with a molecule, e.g., for conjugation to a carrier. In one embodiment, the derivatized LPA is thiolated LPA. In one embodiment, the derivatized LPA is derivatized C12 or C18 LPA. In one embodiment, the thiolated LPA is conjugated via a crosslinker, e.g., a bifunctional crosslinker such as IOA or SMCC, to a carrier, which may be a protein. It may be useful to conjugate the LPA in this way to a protein or other carrier that is immunogenic in the species to be immunized, e.g., keyhole limpet hemocyanin (KLH), serum albumin (including bovine serum albumin or BSA), bovine thyroglobulin, or soybean trypsin inhibitor, using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Non-protein carriers (e.g., colloidal gold) are also known in the art for use in antibody production.

The derivatized or derivatized and conjugated LPA may be used as an immunogen to generate anti-LPA antibodies (polyclonal and/or monoclonal). The derivatized or derivatized and conjugated LPA may also be used in the methods of the invention, particularly in diagnostic methods.

2. Research and Diagnostic Uses for Derivatized LPA

The derivatized LPAs described above may be used to detect and/or purify anti-LPA antibodies and may be conjugated to a carrier as described above. The derivatives and conjugates may be conjugated to a solid support for use in diagnostic methods, including clinical diagnostic methods. For example, detection and/or quantitation of LPA antibodies, particularly autoantibodies, may be used in diagnosing various medical conditions in LPA plays a role. Quantitation of LPA antibodies is also useful in a clinical setting to detect and/or diagnose diseases and conditions characterized by aberrant levels of LPA, or to evaluate dosing, halflife and drug levels, or patient response, after treatment with, e.g., an anti-LPA antibody such as those described herein. Derivatized LPA made as described herein bears a reactive group that does not disable the epitope by which Lpath's anti-LPA antibodies recognize LPA. Thus, the derivatized LPA may be used as part of an assay method or kit that relies on anti-LPA antibodies for detection and/or quantitation of LPA. Derivatized LPA may also be used to allow attachment of a label (radiolabel or other label) to the LPA, for use in scintillation proximity assays (SPA) or other assays.

In one embodiment, the derivatized LPA conjugate (for example, thiolated LPA conjugated to BSA or KLH) is used as laydown material in ELISAs which are used to detect anti-LPA antibodies. In one embodiment the LPA is thiolated C12 LPA or thiolated C18 LPA conjugated to BSA. This embodiment is useful, for example, as laydown material (to coat the plate) in ELISA assays for detection of LPA. For example, in an LPA competitive ELISA, the plate is coated with derivatized and/or derivatized and conjugated LPA. A set of one or more LPA standards and one or more samples (e.g., serum or cell culture supernatant) is mixed with the mouse anti-LPA antibody and added to the derivitized-LPA-coated plate. The antibody competes for binding to either plate-bound LPA or LPA in the sample or standard. Following incubation and several ELISA steps, the absorbance at 450 nm is measured and the LPA concentration in the samples is determined by comparison to the standard curve.

The derivatized or derivatized and conjugated LPA may also be coupled to a solid support (e.g., resin or other column matrix, beads, membrane, plate) and used to isolate and/or purify anti-LPA antibodies, e.g., from blood or serum. Such anti-LPA antibodies may be newly generated antibodies (e.g., mammalian monoclonal or polyclonal antibodies to LPA) or may be native human anti-LPA antibodies.

Thus both derivatized LPA and derivatized and conjugated LPA are useful for research and in clinical diagnostics. In one embodiment, derivatized or derivatized and conjugated LPA is used in kits and methods for detection of neurotrauma by measurement of LPA in patient samples. In one embodiment, these kits and methods also employ an antibody which specifically binds LPA.

B. Anti-LPA Agents, Including Anti-LPA Antibodies

1. Introduction

Effective inhibitors of LPA for therapeutic use have not been identified prior to Lpath's development of highly specific and potent antibodies to LPA. An alternative approach is the inhibition of autotaxin (ATX), a secreted nucleotide pyrophosphatase/phosphodiesterase that functions as a lysophospholipase D to produce LPA. The ATX-LPA signaling axis has been implicated in angiogenesis, chronic inflammation, fibrotic diseases and tumor progression, making this system an attractive target for therapy, but again, suitably potent and selective nonlipid inhibitors of ATX are currently not available. Inhibitors of LPA receptors such as the selective LPA1 receptor antagonist AM966 (Swaney et al. Br J Pharmacol. 2010 August; 160(7): 1699-1713) have also been tried as treatments for LPA-associated disease, particularly fibrosis. It is believed that direct neutralization of LPA itself is a more straightforward and favorable approach than inhibition of LPA synthesis or of one or more of the multiple LPA receptors. Thus compounds that bind LPA tightly and specifically are desired for use as therapeutics and in detection and diagnostics.

2. Disease Associations of LPA and Therapeutic Uses for Anti-LPA Agents

LPA has been associated with a number of diseases and disorders. For review, see Gardell, et al. (2006), Trends Mol Med. 12(2):65-75, and Chun J. and Rosen, H., (2006) Curr. Pharma. Design 12:161-171. These include autoimmune disorders such as diabetes, multiple sclerosis and scleroderma; fibrotic diseases and conditions; hyperproliferative disorders including cancer; disorders associated with angiogenesis and neovascularization; obesity; neurodegenerative diseases including Alzheimer's disease; schizophrenia, immune-related disorders such as transplant rejection and graft-vs.-host disease, and others. Additional descriptions regarding LPA in disease and anti-LPA agents, particularly antibodies, in treatment and prevention of disease may be found, e.g., in U.S. patent application publication numbers: 20090136483, 20080145360, 20100034814 and 20110076269, all of which are commonly owned with the instant invention and are incorporated herein by reference in their entirety.

a. Hyperproliferative Disorders

LPA-associated hyperproliferative disorders include neoplasias, disorders associated with endothelial cell proliferation, and disorders associated with fibrogenesis. Most often, the neoplasia will be a cancer. Typical disorders associated with endothelial cell proliferation are angiogenesis-dependent disorders, for example, cancers caused by a solid tumors, hematological tumors, and age-related macular degeneration. Disorders associated with fibrogenesis include those than involve aberrant cardiac remodeling, such as cardiac failure.

Cancer is now primarily treated with one or a combination of three types of therapies, surgery, radiation, and chemotherapy. Surgery involves the bulk removal of diseased tissue. While surgery is sometimes effective in removing tumors located at certain sites, for example, in the breast, colon, and skin, it cannot be used in the treatment of tumors located in other areas, such as the backbone, nor in the treatment of disseminated neoplastic conditions such as leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. Current therapeutic agents thus usually involve significant drawbacks for the patient in the form of toxicity and severe side effects. Therefore, many groups have recently begun to look for new approaches to fighting the war against cancer. These new so-called “innovative therapies” include gene therapy and therapeutic proteins such as monoclonal antibodies.

The first monoclonal antibody used in the clinic for the treatment of cancer was Rituxan (rituximab) which was launched in 1997, and has demonstrated the utility of monoclonal antibodies as therapeutic agents. Thus, not surprisingly, twenty monoclonal antibodies have since been approved for use in the clinic, including nine that are prescribed for cancer. The success of these products, as well as the reduced cost and time to develop monoclonal antibodies as compared with small molecules has made monoclonal antibody therapeutics the second largest category of drug candidates behind small molecules. Further, the exquisite specificity of antibodies as compared to small molecule therapeutics has proven to be a major advantage both in terms of efficacy and toxicity. Consequently, monoclonal antibodies are poised to become a major player in the treatment of cancer and they are estimated to capture an increasing share of the cancer therapeutic market. Generally therapeutic mAbs are targeted to proteins; only recently has it been feasible to raise mAbs to bioactive lipids (for example, antibodies to S1P, see Applicants' US Patent Application Publication No. 20070148168).

The identification of extracellular mediators that promote tumor growth and survival is a critical step in discovering therapeutic interventions that will reduce the morbidity and mortality of cancer. As described below, LPA is considered to be a pleiotropic, tumorigenic growth factor. LPA promotes tumor growth by stimulating cell proliferation, cell survival, and metastasis. LPA also promotes tumor angiogenesis by supporting the migration and survival of endothelial cells as they form new vessels within tumors. Taken together, LPA initiates a proliferative, pro-angiogenic, and anti-apoptotic sequence of events contributing to cancer progression. Thus, therapies that modulate, and, in particular, reduce LPA levels in vivo will be effective in the treatment of cancer.

Typically, methods for treating or preventing a hyperproliferative disorder such as cancer involve administering to a subject, such as a human subject or patient, an effective amount of each of an anti-LPA agent, such as an anti-LPA antibody, or a plurality of different agent species, and a cytotoxic agent. Cytotoxic agents include chemotherapeutic drugs.

A related method is intended to reduce toxicity of a therapeutic regimen for treatment or prevention of a hyperproliferative disorder. Such methods comprise administering to a subject, such as a human subject or patient, suffering from a hyperproliferative disorder an effective amount of an anti-LPA agent, such as an anti-LPA antibody, or a plurality of different agents, before, during, or after administration of a therapeutic regimen intended to treat or prevent the hyperproliferative disorder. It is believed that by using anti-LPA agents to sensitize cells, e.g., cancer cells, to chemotherapeutic drugs, efficacy can be achieved at lower doses and hence lower toxicity due to chemotherapeutic drugs.

Yet another aspect of the invention concerns methods of enhancing a survival probability of a subject treated for a hyperproliferative disorder by administering to a subject suffering from a hyperproliferative disorder an anti-LPA agent, such as an anti-LPA antibody, or a plurality of different agent species, before, during, or after administration of a therapeutic regimen intended to treat or prevent the hyperproliferative disorder to enhance the subject's survival probability.

1. Fibrosis, Wound Healing and Scar Formation

Fibroblasts, particularly myofibroblasts, are key cellular elements in scar formation in response to cellular injury and inflammation (Tomasek et al. (2002), Nat Rev Mol Cell Biol, vol 3: 349-63, and Virag and Murry (2003), Am J Pathol, vol 163: 2433-40). Collagen gene expression by myofibroblasts is a hallmark of remodeling and necessary for scar formation (Sun and Weber (2000), Cardiovasc Res, vol 46: 250-6, and Sun and Weber (1996), J Mol Cell Cardiol, vol 28: 851-8).

Fibrosis can be described as the formation or development of excess or aberrant fibrous connective tissue in an organ or tissue as part of a pathological reparative or reactive process, in contrast to normal wound healing or development. The most common forms of fibrosis are: liver, lung, kidney, skin, uterine and ovarian fibroses. Some conditions, such as scleroderma, sarcoidosis and others, are characterized by fibrosis in multiple organs and tissues.

Recently, the bioactive lysophospholipid lysophosphatidic acid (LPA) has been recognized for its role in tissue repair and wound healing. Watterson et al., Wound Repair Regen. (2007) 15:607-16. As a biological mediator, LPA has been recognized for its role in tissue repair and wound healing (Watterson, 2007). In particular, LPA is linked to pulmonary and renal inflammation and fibrosis. LPA is detectable in human bronchioalveolar lavage (BAL) fluids at baseline and its expression increases during allergic inflammation Georas, S. N. et al. (2007) Clin Exp Allergy. (2007) 37: 311-22. Furthermore, LPA promotes inflammation in airway epithelial cells. Barekzi, E. et al (2006) Prostaglandins Leukot Essent Fatty Acids. 74:357-63. Recently, pulmonary and renal fibrosis have been linked to increased LPA release and signaling though the LPA type 1 receptor (LPA₁). LPA levels were elevated in bronchialveolar lavage (BAL) samples from IPF patients and bleomycin-induced lung fibrosis in mice was dependent on activation of LPA₁. Tager et al., (2008) Proc Am Thorac Soc. 5: 363. (2008) Following unilateral ureteral obstruction in mice, tubulointerstitial fibrosis was reduced in LPA₁ knock-out mice and pro-fibrotic cytokine expression was attenuated in wild-type mice treated with an LPA₁ antagonist. J. P. Pradere et al., (2007) J. Am. Soc. Nephrol. 18:3110-3118. LPA has been shown to have direct fibrogenic effects in cardiac fibroblasts by stimulating collagen gene expression and proliferation. Chen, et al. (2006) FEBS Lett. 580:4737-45. Combined, these studies demonstrate a role for LPA in tissue repair and fibrosis, and identify bioactive lipids as a previously unrecognized class of targets in the treatment of fibrotic disorders.

Examples of fibrotic disorders include scleroderma, pulmonary fibrosis, liver fibrosis, renal fibrosis, uterine fibrosis, fibrosis of the skin, and cardiac fibrosis. Agents that reduce the effective concentration of LPA, such as Lpath's anti-LPA mAb, are believed to be useful in methods for treating diseases and conditions characterized by aberrant fibrosis.

b. Cardiovascular and Cerebrovascular Disorders

Because LPA is involved in fibrogenesis and wound healing of liver tissue (Davaille et al., J. Biol. Chem. 275:34268-34633, 2000; Ikeda et al., Am J. Physiol. Gastrointest. Liver Physiol 279:G304-G310, 2000), healing of wounded vasculatures (Lee et al., Am. J. Physiol. Cell Physiol. 278:C612-C618, 2000), and other disease states, or events associated with such diseases, such as cancer, angiogenesis and inflammation (Pyne et al., Biochem. J. 349:385-402, 2000), the compositions and methods of the disclosure may be applied to treat not only these diseases but cardiac diseases as well, particularly those associated with tissue remodeling. LPA have some direct fibrogenic effects by stimulating collagen gene expression and proliferation of cardiac fibroblasts. Chen, et al. (2006) FEBS Lett. 580:4737-45.

c. Obesity and Diabetes

Autotaxin, a phospholipase D responsible for LPA synthesis, has been found to be secreted by adipocytes and its expression is up-regulated in adipocytes from obese-diabetic db/db mice as well as in massively obese women subjects and human patients with type 2 diabetes, independently of obesity (Ferry et al. (2003) JBC 278:18162-18169; Boucher et al. (2005) Diabetologia 48:569-577, cited in Pradere et al. (2007) BBA 1771:93-102. LPA itself has been shown to influence proliferation and differentiation of preadipocytes. Pradere et al., 2007. Together this suggests a role for anti-LPA agents in treatment of obesity and diabetes.

d. Pain

A significant role of LPA in the development of pain, including neuropathic pain, was established using various pharmacological and genetic approaches. LPA is responsible for long-lasting mechanical allodynia and thermal hyperalgesia as well as demyelination and upregulation of pain-related proteins through the LPA1 receptor. In addition, intrathecal injections of LPA induce behavioral, morphological and biochemical changes such as prolonged sensitivity to pain stimuli accompanied by demyelination of dorsal roots, similar to those observed after nerve ligation. Fujita, R., Kiguchi, N. & Ueda, H. (2007) Neurochem Int 50, 351-5. Wild-type animals with nerve injury develop behavioral allodynia and hyperalgesia paralleled by demyelination in the dorsal root and increased expression of both the protein kinase C isoform within the spinal cord dorsal horn and the 21 calcium channel subunit in dorsal root ganglia. It has been demonstrated that mice lacking the LPA1 receptor gene (Ipa1−/− mice) lose nerve injury-induced neuropathic pain behaviors and phenomena. Inoue, M. et al. (2004) Nat Med 10, 712-8. Heterozygous mutant mice for the autotaxin gene (atx+/−) showed approximately 50% recovery of nerve injury-induced neuropathic pain. The hyperalgesia was completely abolished in both Ipa1−/− and atx+/− mice. Furthermore, inhibitors of Rho and Rho kinase signaling pathways also prevented neuropathic pain. Mueller, B. K., Mack, H. & Teusch, N. (2005) Nat Rev Drug Discov 4, 387-98. Therefore, targeting LPA biosynthesis and/or LPA1 receptor may represent a novel approach to mitigating nerve-injury-induced neuropathic pain

At the cellular level, LPA is a potent inducer of morphological changes in neuronal and glial cells 66, 151-155. Kingsbury, M. A., et al. (2003) Nat Neurosci 6, 1292-9; Jalink, K. et al., (1993) Cell Growth Differ 4, 247-55; Tigyi, G. & Miledi, R. (1992) J Biol Chem 267, 21360-7 (1992); Fukushima, N. et al. (2000) Dev Biol 228, 6-18; Yuan, X. B. et al. (2003) Nat Cell Biol 5, 38-45; Fukushima, N., et al. (2007) Neurochem Int 50, 302-7.

In primary astrocytes, as well as in glioma-derived cell lines, LPA causes reversal of process outgrowth (‘stellation’), a process directed by active RhoA and accompanied by reassembly and activation of focal adhesion proteins. Ramakers, G. J. & Moolenaar, W. H. (1998). Exp Cell Res 245, 252-62. A role for LPA in myelination is also suggested by the finding that LPA promotes cell-cell adhesion and survival in Schwann cells. Weiner, J. A., et al. (2001) J Neurosci 21, 7069-78; Ramer, L. M. et al (2004) J Neurosci 24, 10796-805.

e. Neurotrauma and CNS Diseases/Conditions

Key components of the LPA pathway are modulated following CNS injury. In the adult mouse, LPA receptors are differentially expressed in the spinal cord and LPA receptors 1-3 (LPA1-3) are strongly upregulated in response to injury. Goldshmit, et al. (2010), Cell Tissue Res. 341:23-32. Examination of LPA receptors expression in the intact uninjured spinal cord showed that LPA1-3 are expressed at low but distinct levels in different areas of the spinal cord. LPA1 is expressed in the central canal by ependymal cells, while LPA2 is expressed in cells immediately surrounding the central canal and at low levels on some astrocytes in the grey matter. LPA3 is expressed at low levels on motor neurons of the ventral horn and throughout the grey matter neuropil. Following SCI, LPA1 is still expressed on a subpopulation of astrocytes near the injury site at 4 days following injury, although its level of expression is increased. LPA2 is expressed by astrocytes, with an upregulation on reactive astrocytes around the lesion site by 2 days, and further increased by 4 days. LPA3 expression remains confined to neurons but is upregulated in a small number of neurons by 2 days, and further increased by 4 days extending its expression to the neuronal processes. This upregulation is observed not only close to the lesion site, but also distal from both sides.

Considering the pleiotropic effects of LPA on most neural cell types, especially on cell morphology, proliferation and survival, together with demonstration of a localized upregulation of LPA1-3 following injury, it is likely that LPA regulates essential aspects of the cellular reorganization following neurotrauma by being a key player in reactive astrogliosis, neural regeneration and axonal re-growth.

Data strongly suggest that neural responses to LPA stimuli are likely to significantly influence the amount of ensuing damage or repair following brain and/or spinal cord injury. Elevated levels of LPA are observed in certain pathological states including brain and spinal cord injury. LPA injections into mouse brain induce astrocyte reactivity at the site of the injury, while in the spinal cord, LPA induces neuropathic pain and demyelination. LPA can stimulate astrocytic proliferation and can promote death of hippocampal neurons. Moreover, LPA mediates microglial activation and is cytotoxic to the neuromicrovascular endothelium.

Following injury, LPA is synthesized in the mouse spinal cord in a model of sciatic nerve ligation (Ma, Uchida et al. 2010) and LPA-like activity is increased in the cerebrospinal fluid following intrathecal injection of autologous blood to mimic cerebral hemorrhage in newborn pigs (Tigyi, et al. (1995), Am J. Physiol. 268:H2048-2055; Yakubu, et al. (1997), Am J. Physiol. 273:R703-709). Normally undetectable, levels of ATX increase in astrocytes neighboring a lesion of the adult rat brain (Savaskan, et al. (2007), Cell Mol. Life Sci. (2007) 64:230-43). In humans, the presence of ATX in cerebrospinal fluid has been demonstrated in multiple sclerosis patients (Hammack, et al. (2004), Mult Scler. 10:245-60 and higher levels of LPA in human plasma might predict silent brain infarction (Li, et al. (2010), Int J Mol Sci. 11:3988-98). Further, in human cerebrospinal fluid from traumatic brain injury (TBI) patients (Farias, et al. (2011), J Trauma. 71:1211-8) increased levels of arachidonic acid, a lipid generated from the hydrolysis of phosphatidic acid into LPA and arachidonic acid, have been reported.

Following injury, hemorrhage, or trauma to the nervous system, levels of LPA within the nervous system are believed to increase to 10 μM. Dottori, et al. [(2008) Stem Cells 26:1146-54] have shown that 10 μM LPA can inhibit neuronal differentiation of human NSC, while lower concentrations do not, suggesting that high levels of LPA within the CNS following injury might inhibit differentiation of NSC toward neurons, thus inhibiting endogenous neuronal regeneration. Modulating LPA signaling may thus have a significant impact in nervous system injury, allowing new potential therapeutic approaches. Antibodies to LPA have now been shown (see examples below) to decrease infarct size, neuroinflammation (including gliogenesis) and neurodegeneration.

LPA and LPA metabolites have now been shown to be a biomarker for neurotrauma, such as TBI, as is shown in the examples below. LPA is elevated in CSF following TBI, and thus can be used diagnostically to indicate the presence of neurotrauma. This allows the development of rapid methods and kits as are described herein, to aid in detecting neurotrauma independently of neurological symptoms. Such methods and kits can be used, for example, by emergency medical personnel, emergency room physicians, and in combat situations, to aid in patient triage. The rapidity of the method allows treatment to begin soon after injury, which is believed to minimize (to the extent possible) the CNS damage that occurs subsequent to the initial injury. Treatment may be with anti-LPA agents such as the antibodies described herein, which have shown efficacy in models of neurotrauma (see examples herein).

In addition to LPA, other markers for neurotrauma are known, and these may be used in combination with LPA in methods and kits for detecting and diagnosing neurotrauma. For example, comparison of albumin levels in CSF and serum can be used to assess BBB disruption, and the astrocytic protein S100B and monomeric transthyretin have been reported as serum markers for BBB disruption. Blyth et al. (2009) J. Neurotrauma 26:1497-1507. A proteomics approach has been used to identify 30 putative prognostic biomarkers for TBI, including cerebellin, FGF-13, glutathione peroxidase 3, serpinA3, murinoglobin, ApoA4, Clusterin/ApoJ, complement proteins C1 QB, C8B and CBG, fibrinogen alpha and beta chains, prothrombin, hemoglobin subunits alpha, beta and delta, hemopexin, and ten immunoglobin (or related) proteins: IGHG, IGK5, EP3-6, LOC100047628, IGHM, IGL3C, IGH2, IGK8, IGG3C, and EALC. Crawford et al. (2012) J. Neurotrauma 29:246-60. The phosphorylated form of the high-molecular-weight neurofilament subunit NF-H (pNF-H), has been reported to be elevated in blood after SCI, with levels reflecting the degree of axonal damage. Hayakawa et al. (2012) 1-4. Serum levels of ubiquitin C-terminal hydrolase (UCH-L1) have been shown to distinguish mild TBI from controls. UCH-L1 is detectable within an hour of injury and levels correlate with Glasgow score, existence of intracranial lesions detectable by CT, and need for neurosurgical intervention. Papa et al. (2012) J. Trauma 72:1335-1344. Glial fibrillary acidic protein (GFAP) is a brain-specific biomarker that is released into the blood following TBI and stroke and is a putative biomarker for these conditions. Schiff, L. et al., Mol Diagn Ther. 2012 Apr. 1; 16(2):79-92 (abstract). Plasma GFAP analysis performed within 4.5 h of symptom onset can differentiate intracranial hemorrhage from ischemic stroke. Foerch et al. (2012) Clinical Chemistry 58: 237-245 and US patent application publication 20060240480. Additional putative protein biomarkers for TBI include SBDP150, SBDP120, MBPlfrag, MAP2, BA0293, S100B, NSA, MMP9, VCAM and IL-12.

In addition to LPA, other lipid biomarkers for neurotrauma also exist, such as LPA metabolites as described herein (e.g., lyso-PAF and LPC), as well as other lipid biomarkers such as 12-hydroxyeicosatetraenoic acid (12-HETE), which has been shown to be elevated in CSF of patients after TBI [Farias et al., (2011) J. Trauma 71:1211-1218]. These lipid biomarkers may also be used alone or in combination for detection and diagnosis of neurotrauma. In one embodiment, levels of LPA and of one or more LPA metabolites are determined in one or more biological samples from a subject to detect and/or diagnose neurotrauma. In another embodiment, levels of LPA and/or levels of an LPA metabolite in addition to levels of 12-HETE are determined in one or more biological samples from a subject to detect and/or diagnose neurotrauma.

Other examples of protein and lipid biomarkers for neurotrauma exist. The methods and kits for detecting and diagnosing neurotrauma as disclosed herein may rely on determination of LPA alone or in combination with detection and/or measurement of one or more additional markers of neurotrauma. In some embodiments, determination of multiple biomarkers is desired.

3. Antibodies to LPA

Polyclonal antiserum against naturally-occurring LPA has been reported in the literature (Chen J H, et al., Bioorg Med Chem Lett. 2000 Aug. 7; 10(15):1691-3). The examples hereinbelow describe the production of monoclonal anti-LPA antibodies with desirable properties from a therapeutic perspective including: (a) binding affinity for LPA and/or its variants, including 18:2, 18:1, 18:0, 16:0, 14:0, 12:0 and 20:4 LPA. Antibody affinities may be determined as described in the examples herein below. Preferably antibodies bind LPA with a high affinity, e.g., a K_{d} value of no more than about 1×10⁻⁷ M; possibly no more than about 1×10⁻⁸ M; and possibly no more than about 5×10⁻⁹ M. In a physiological context, it is preferable for an antibody to bind LPA with an affinity that is higher than the LPA's affinity for an LPA receptor. It will be understood that this need not necessarily be the case in a nonphysiological context such as a diagnostic assay.

Aside from antibodies with strong binding affinity for LPA, it may also be desirable to select chimeric, humanized or variant antibodies which have other beneficial properties from a therapeutic perspective. For example, the antibody may be one that reduces scar formation or alters tumor progression. One assay for determining the activity of the anti-LPA antibodies is ELISA. Preferably the humanized or variant antibody fails to elicit an immunogenic response upon administration of a therapeutically effective amount of the antibody to a human patient. If an immunogenic response is elicited, preferably the response will be such that the antibody still provides a therapeutic benefit to the patient treated therewith.

More information about antibodies to LPA, including antigen-binding antibody fragments and variants, can be found in applicant's patent applications, e.g., US Patent Application Publication Nos: 20090136483, 20080145360, 20100034814 and 20110076269, all of which are commonly owned with the instant invention and are incorporated herein by reference in their entirety, and in the examples below. Antibodies to LPA may be polyclonal or monoclonal, and may be humanized. Isolated nucleic acid encoding the anti-LPA antibody, vectors and host cells comprising the nucleic acid, and recombinant techniques for the production of the antibody are also described in the above patent applications.

a. Pharmaceutical Formulations, Dosing and Routes of Administration

One way to control the amount of undesirable LPA in a patient is by providing a composition that comprises one or more anti-LPA antibodies to bind one or more LPAs, thereby acting as therapeutic “sponges” that reduce the level of free LPA. When a compound is stated to be “free,” the compound is not in any way restricted from reaching the site or sites where it exerts its undesirable effects. Typically, a free compound is present in blood and tissue, which either is or contains the site(s) of action of the free compound, or from which a compound can freely migrate to its site(s) of action. A free compound may also be available to be acted upon by any enzyme that converts the compound into an undesirable compound.

Anti-LPA antibodies may be formulated in a pharmaceutical composition that is useful for a variety of purposes, including the treatment of diseases, disorders or physical trauma. Pharmaceutical compositions comprising one or more anti-LPA antibodies may be incorporated into kits and medical devices for such treatment. Medical devices may be used to administer the pharmaceutical compositions to a patient in need thereof, and according to one embodiment, kits are provided that include such devices. Such devices and kits may be designed for routine administration, including self-administration, of the pharmaceutical compositions. Such devices and kits may also be designed for emergency use, for example, in ambulances or emergency rooms, or during surgery, or in activities where injury is possible but where full medical attention may not be immediately forthcoming (for example, hiking and camping, or combat situations).

Therapeutic formulations of the antibody are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished for instance by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

For therapeutic applications, the anti-LPA agents, e.g., antibodies, are administered to a mammal, preferably a human, in a pharmaceutically acceptable dosage form such as those discussed above, including those that may be administered to a human intravenously as a bolus or by continuous infusion over a period of time, or by intramuscular, intraperitoneal, intra-cerebrospinal, subcutaneous, intra-articular, intrasynovial, intrathecal, oral, topical, intranasal or inhalation routes. For CNS applications, intracerebrosponal, intrathecal and intranasal administration may be particularly useful. Although the blood-brain barrier (BBB) is impermeable to most drugs, intranasal delivery allows efficient drug delivery into the CNS; this is believed to occur via the rostral migratory stream, trigeminal nerve and/or olfactory nerve. Scranton et al. (2011) PLoS ONE 6:e18711. For a review of intranasal administration, including routes and devices, see Dhuria et al. (2010) J Pharm Sci. 99(4):1654-73. It may be preferable to administer the drug intranasally into the upper third of the nasal cavity (U.S. Pat. No. 6,313,093, Frey, W H). In addition to these targeted CNS routes, the possibility of CNS administration through systemic or other routes also exists in patients with neurotrauma because the BBB is often compromised for a window of time following neurotrauma.

For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 .mu.g/kg to about 50 mg/kg (e.g., 0.1-20 mg/kg) of antibody is an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging. Detection methods using the antibody to determine LPA levels in bodily fluids or tissues may be used in order to optimize patient exposure to the therapeutic antibody.

According to another embodiment, the composition comprising an agent, e.g, a mAb, that interferes with LPA activity is administered as a monotherapy, while in other preferred embodiments, the composition comprising the agent that interferes with LPA activity is administered as part of a combination therapy. In some cases the effectiveness of the antibody in preventing or treating disease may be improved by administering the antibody serially or in combination with another agent that is effective for those purposes, such as a chemotherapeutic drug for treatment of cancer. In other cases, the anti-LPA agent may serve to enhance or sensitize cells to chemotherapeutic treatment, thus permitting efficacy at lower doses and with lower toxicity. Preferred combination therapies include, in addition to administration of the composition comprising an agent that interferes with LPA activity, delivering a second therapeutic regimen selected from the group consisting of administration of a chemotherapeutic agent, radiation therapy, surgery, and a combination of any of the foregoing.

Such other agents may be present in the composition being administered or may be administered separately. Also, the antibody is suitably administered serially or in combination with the other agent or modality, e.g., chemotherapeutic drug or radiation for treatment of cancer.

b. Research and Diagnostic, Including Clinical Diagnostic, Uses for Anti-LPA Agents

Anti-LPA agents, e.g., aptamers, receptor fragments, small molecules and antibodies, are molecules which specifically bind LPA. As such they may be used to detect and/or purify LPA, e.g., from bodily fluid(s). For use of anti-LPA antibodies as affinity purification agents, the antibodies are immobilized on a solid support such as beads, a Sephadex resin or filter paper, using methods well known in the art. The immobilized antibody (or other anti-LPA detection reagent) is contacted with a sample containing the LPA to be purified, and thereafter the support is washed with a suitable solvent that will remove substantially all the material in the sample except the LPA, which is bound to the immobilized antibody. Finally, the support is washed with another suitable solvent, such as glycine buffer, for instance between pH 3 to pH 5.0, that will release the LPA from the antibody.

Anti-LPA antibodies are useful in diagnostic assays for LPA, e.g., detecting its presence in specific cells, tissues, or bodily fluids. Such diagnostic methods may be useful in diagnosis, e.g., of a hyperproliferative disease or disorder. Thus, clinical diagnostic uses as well as research uses are comprehended by the invention. In these methods, the anti-LPA antibody is preferably attached to a solid support, e.g., bead, column, plate, gel, filter, membrane, etc.

For diagnostic applications, the antibody may be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

(c) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and (iii) .beta.-D-galactosidase (.beta.-D-Gal) with a chromogenic substrate (e.g., p-nitrophenyl-β-D-galactosidase) or fluorogenic substrate 4-methylumbelliferyl-.beta.-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In another embodiment, the anti-LPA antibody need not be labeled, and the presence thereof can be detected, e.g., using a labeled antibody which binds to the anti-LPA antibody.

The antibodies may be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). ELISA assays (competitive or direct) using the anti-LPA antibodies are useful for detecting LPA and assessing its binding and antigen specificity. An LPA ELISA kit incorporating applicant's anti-LPA antibody is commercially available from Echelon Biosciences, Salt Lake City Utah (cat no. K-2800).

Competitive binding assays rely on the ability of a labeled standard to compete with the test sample analyte for binding with a limited amount of antibody. The amount of LPA in the test sample is inversely proportional to the amount of standard that becomes bound to the antibodies. To facilitate determining the amount of standard that becomes bound, the antibodies generally are insoluble before or after the competition, so that the standard and analyte that are bound to the antibodies may conveniently be separated from the standard and analyte that remain unbound.

Sandwich assays involve the use of two antibodies, each capable of binding to a different immunogenic portion, or epitope, of the protein to be detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the blood or tissue sample may be fresh or frozen or may be embedded in paraffin and fixed with a preservative such as formalin, for example.

The antibodies may also be used for in vivo diagnostic assays. The antibody may be radiolabeled (such as with ¹¹¹In, ⁹⁹Tc, ¹⁴C, ¹³¹I, ¹²⁵I, ³H, ³²P, or ³⁵S) so that the bound target molecule can be localized using immunoscintillography.

c. Kits for Detection and Diagnosis of LPA-Associated Diseases or Conditions

As a matter of convenience for methods of detecting and diagnosing neurotrauma, particularly for emergency medical use, antibodies to LPA can be provided in a kit, e.g., a packaged combination of reagents in predetermined amounts with instructions for performing the method. In some embodiments the kit also includes patient sample collection equipment, e.g., syringes, for collecting, e.g., blood or CSF, vials for collection of bodily fluids such as urine, tears, saliva, etc. In one embodiment the kit comprises materials for an antibody-based assay for detection and quantitation of LPA, and preferably contains means and materials (such as standards) for quantitation of the LPA in the patient sample to determine whether LPA levels are elevated to levels indicative of neurotrauma.

In one embodiment, the LPA assay is performed on a solid support that is compact and portable in a kit, such as a microtiter plate. In one embodiment, the assay for detecting and quantitating LPA for diagnosis of neurotrauma is an ELISA assay. In one embodiment, this assay uses both anti-LPA antibody and derivatized LPA as described herein. The derivatized LPA conjugate (e.g., thiolated LPA conjugated to BSA or KLH) may be used as laydown material (to coat the plate) in ELISA kits that are used to detect anti-LPA antibodies. As one example, in an LPA competitive ELISA kit, the plate (provided) is coated with derivatized and/or derivatized and conjugated LPA. A set of one or more LPA standards (generally provided in the kit) and one or more samples (e.g., urine, blood, serum, cells or tissue) is mixed with the mouse anti-LPA antibody and added to the derivitized-LPA-coated plate. The antibody competes for binding to either plate-bound LPA or LPA in the sample or standard. Following incubation and several ELISA steps (instructions and reagents for which are provided in the kit), the LPA concentration in the samples is determined by comparison to the standard curve, for example, using a colorimetric assay. In one nonlimiting embodiment the LPA used for laydown material in the ELISA kit is thiolated C12 LPA or thiolated C18 LPA conjugated to BSA. The antibody used in the kit may be a polyclonal or monoclonal antibody, preferably a monoclonal antibody.

A kit incorporating a derivatized and conjugated LPA and an anti-LPA antibody, both of which were developed by Lpath Inc., is commercially available from Echelon Biosciences, Inc., Salt Lake City, Utah (Lysophosphatidic Assay Kit, Cat. No. K-2800).

As a matter of convenience, anti-LPA antibodies (or antigen-binding fragments thereof) can be provided in a kit, for example, a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents that substantially optimize the sensitivity of the assay. Particularly, the reagents may be provided as dry powders, usually lyophilized, including excipients that on dissolution will provide a reagent solution having the appropriate concentration. The kit may also contain materials for convenient and safe patient sample collection—sterile gloves, sterilizing wipes, washes, rinses or swabs for ensuring patient safety and/or sterile sample collection, etc.

In one embodiment, the kit uses a lateral flow test [immunochromatographic strip (ICS) or lateral flow immunoassay (LFIA)] format which is widely used for rapid diagnostics and has the advantage of being easiest to use, particularly in the field, and can test for several analytes (e.g., LPA and one or more additional biomarkers) at once. Such lateral flow kits and methods are particularly suited to point-of-care testing. In one embodiment, a sample of blood or urine is tested using a lateral flow test to detect LPA and/or an LPA metabolite(s) to detect and diagnose neurotrauma.

In some embodiments, the diagnostic kit also contains therapeutic materials, including an anti-LPA antibody or antigen-binding fragment thereof, for treatment of neurotrauma. This kit is intended to provide rapid neuroprotective treatment of a patient who has been determined, using the diagnostic portion of the kit as described above, to have sustained neurotrauma. The therapeutic portion of the kit preferably contains materials for patient dosing, such as sterile syringes, sterile gloves, etc. along with dosing information and necessary materials for dissolving and/or diluting the antibody, if needed. In some embodiments the kit contains solutions and devices for intranasal administration of the antibody.

The great advantage of the kits as described herein is the rapid diagnosis of neurotrauma, which allows treatment during the critical window of time during which neuroprotection is possible, before the second phase of brain injury causes maximal damage, and possibly while the BBB is still compromised. In some cases the kit also provides necessary materials for treatment, allowing treatment to begin immediately upon diagnosis, even before reaching an emergency room setting. Because there is no single TBI symptom or pattern of symptoms that characterize mild TBI, for example, a rapid screening test, ideally one (such as a kit described herein) that can be used in the field or in a rescue vehicle. Undiagnosed and untreated TBI presents a risk because some signs and symptoms may be delayed from days to months after injury, and may have significant impact on the patient's physical, emotional, behavioral, social, or family status if untreated, and may result in a functional impairment.

d. Other Articles of Manufacture

In another aspect, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the anti-sphingolipid antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The invention will be better understood by reference to the following Examples, which are intended to merely illustrate the best mode now known for practicing the invention. The scope of the invention is not to be considered limited thereto.

### Examples

The invention will be further described by reference to the following detailed examples. These Examples are in no way to be considered to limit the scope of the invention in any manner.

### Example 1: Synthetic Scheme for Making a Representative Thiolated Analog of S1P

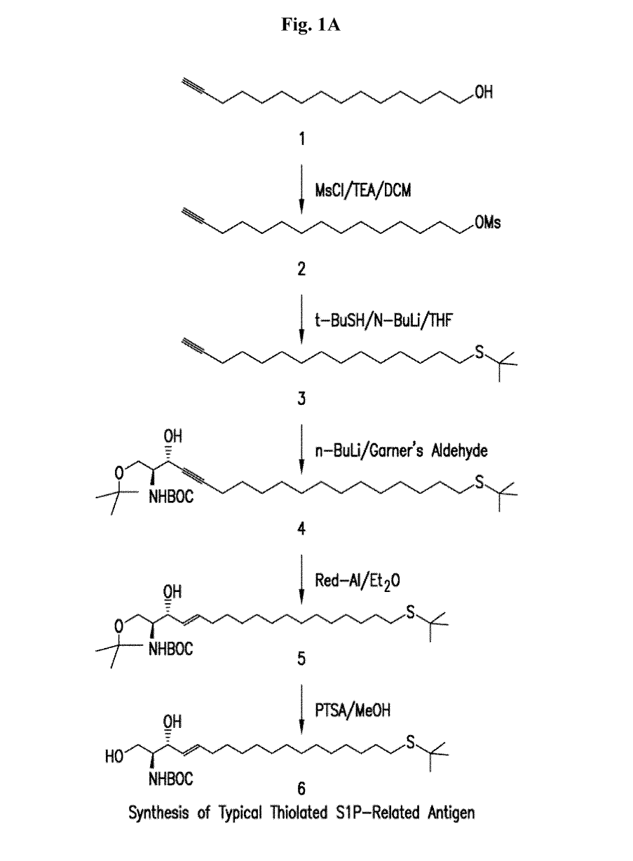

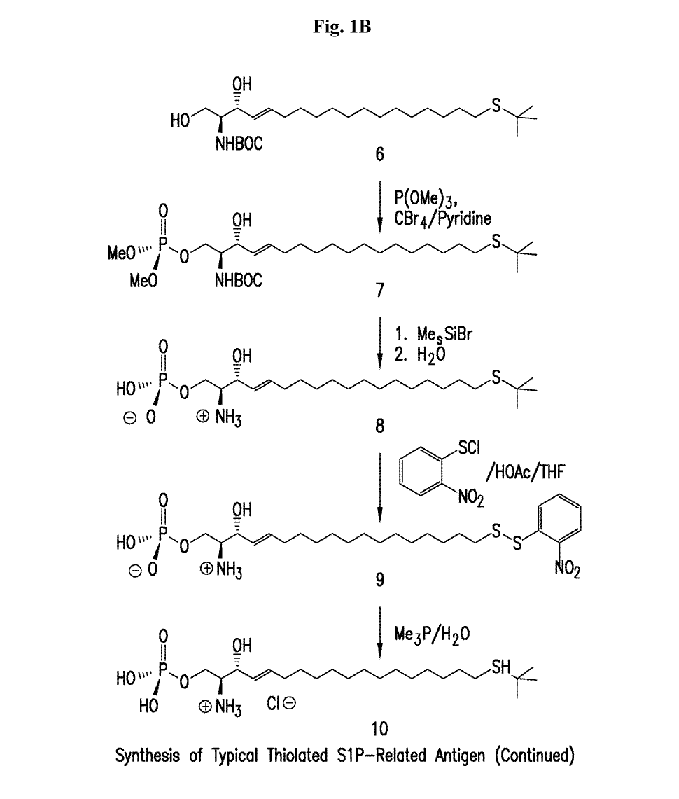

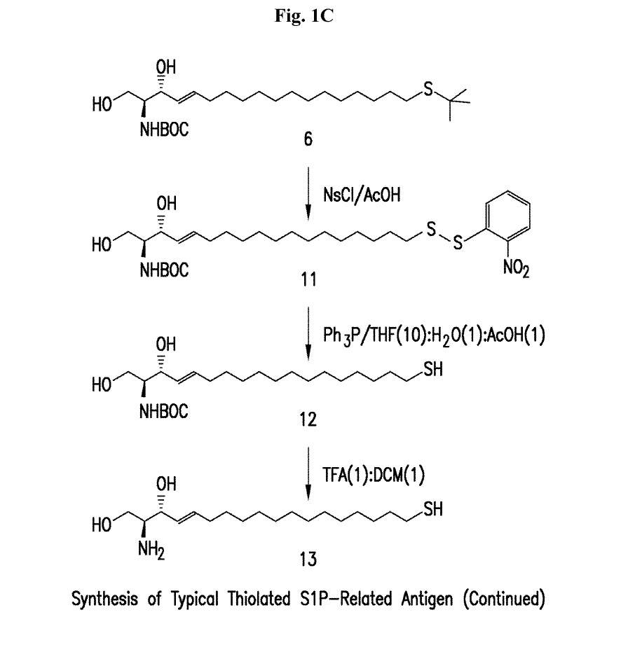

The synthetic approach described in this example results in the preparation of an antigen by serial addition of structural elements using primarily conventional organic chemistry. A scheme for the approach described in this example is provided in FIG. 1, and the compound numbers in the synthetic description below refer to the numbered structures in FIG. 1.

This synthetic approach began with the commercially available 15-hydroxyl pentadecyne, 1, and activation by methyl sulphonyl chloride of the 15-hydroxy group to facilitate hydroxyl substitution to produce the sulphonate, 2. Substitution of the sulphonate with t-butyl thiol yielded the protected thioether, 3, which was condensed with Garner's aldehyde to produce 4. Gentle reduction of the alkyne moiety to an alkene (5), followed by acid catalyzed opening of the oxazolidene ring yielded S-protected and N-protected thiol substituted sphingosine, 6. During this last step, re-derivatization with di-t-butyl dicarbonate was employed to mitigate loss of the N—BOC group during the acid-catalyzed ring opening.

As will be appreciated, compound 6 can itself be used as an antigen for preparing haptens to raise antibodies to sphingosine, or, alternatively, as starting material for two different synthetic approaches to prepare a thiolated S1P analog. In one approach, compound 6 phosphorylation with trimethyl phosphate produced compound 7. Treatment of compound 7 with trimethylsilyl bromide removed both methyl groups from the phosphate and the t-butyloxycarbonyl group from the primary amine, leaving compound 8 with the t-butyl group on the sulfur as the only protecting group. To remove this group, the t-butyl group was displaced by NBS to form the disulfide, 9, which was then reduced to form the thiolated S1P analog, 10.

Another approach involved treating compound 6 directly with NBSCl to form the disulfide, 11, which was then reduced to form the N-protected thiolated S1P analog, 12. Treatment of this compound with mild acid yielded the thiolated sphingosine analog, 13, which can be phosphorylated enzymatically with, e.g., sphingosine kinase, to yield the thiolated S1P analog, 10.

Modifications of the presented synthetic approach are possible, particularly with regard to the selection of protecting and de-protecting reagents, e.g., the use of trimethyl disulfide triflate described in Example 3 to de-protect the thiol.

Compound 2.

DCM (400 mL) was added to a 500 mL RB flask charged with 1 (10.3 g, 45.89 mmol), and the resulting solution cooled to 0° C. Next, TEA (8.34 g, 82.60 mmol, 9.5 mL) was added all at once followed by MsCl (7.88 g, 68.84 mmol, 5.3 mL) added drop wise over 10 min. The reaction was allowed to stir at RT for 0.5 h or until the disappearance of starting material (R_{f}=0.65, 5:1 hexanes:EtOAc). The reaction was quenched with NH₄Cl (300 mL) and extracted (2×200 mL) DCM. The organic layers were dried over MgSO₄, filtered and the filtrate evaporated to a solid (13.86 g, 99.8% yield). ¹H NMR (CDCl₃) δ 4.20 (t, J=6.5 Hz, 2H), 2.98 (s, 3H), 2.59 (td, J=7 Hz, 3 Hz, 2H), 1.917 (t, J=3 Hz, 1H), 1.72 (quintet, J=7.5 Hz, 2H), 1.505 (quintet, J=7.5 Hz, 2H), 1.37 (br s, 4H), 1.27 (br s, 14H). ¹³C{¹H} NMR (CDCl₃) δ 85.45, 70.90, 68.72, 46.69, 38.04, 30.22, 30.15, 30.14, 30.07, 29.81, 29.76, 29.69, 29.42, 29.17, 26.09, 19.06, 9.31. The principal ion observed in a HRMS analysis (ES-TOF) of compound 2 was m/z=325.1804 (calculated for C₁₆H₃₀O₃S: M+Na⁺ 325.1808).

Compound 3.

A three-neck 1 L RB flask was charged with t-butylthiol (4.54 g, 50.40 mmol) and THF (200 mL) and then placed into an ice bath. n-BuLi (31.5 mL of 1.6 M in hexanes) was added over 30 min. Next, compound 2 (13.86 g, 45.82 mmol), dissolved in THF (100 mL), was added over 2 min. The reaction is allowed to stir for 1 hour or until starting material disappeared (R_{f}=0.7, 1:1 hexanes/EtOAc). The reaction was quenched with saturated NH₄Cl (500 mL) and extracted with EtO₂ (2×250 mL), dried over MgSO₄, filtered, and the filtrate evaporated to yield a yellow oil (11.67 g, 86% yield). ¹H NMR (CDCl₃) δ 2.52 (t, J=7.5 Hz, 2H), 2.18 (td, J=7 Hz, 2.5 Hz, 2H), 1.93 (t, J=2.5 Hz, 1H), 1.55 (quintet, J=7.5 Hz, 2H), 1.51 (quintet, J=7 Hz, 2H), 1.38 (br s, 4H), 1.33 (s, 9H), 1.26 (s, 14H). ¹³C{¹H} NMR (CDCl₃) δ 85.42, 68.71, 68.67, 54.07, 42.37, 31.68, 30.58, 30.28, 30.26, 30.19, 30.17, 29.98, 29.78, 29.44, 29.19, 29.02, 19.08.

Compound 4.

A 250 mL Schlenk flask charged with compound 3 (5.0 g, 16.85 mmol) was evacuated and filled with nitrogen three times before dry THF (150 mL) was added. The resulting solution cooled to −78° C. Next, n-BuLi (10.5 mL of 1.6M in hexanes) was added over 2 min. and the reaction mixture was stirred for 18 min. at −78° C. before the cooling bath was removed for 20 min. The dry ice bath was returned. After 15 min., Garner's aldeyde (3.36 g, 14.65 mmol) in dry THF (10 mL) was then added over 5 min. After 20 min., the cooling bath was removed. Thin layer chromatography (TLC) after 2.7 hr. showed that the Garner's aldehyde was gone. The reaction was quenched with saturated aqueous NH₄Cl (300 mL) and extracted with Et₂O (2×250 mL). The combined Et₂O phases were dried over Na₂SO₄, filtered, and the filtrate evaporated to give crude compound 4 and its syn diastereomer (not shown in FIG. 1) as a yellow oil (9.06 g). This material was then used in the next step without further purification.

Compound 5.

To reduce the triple bond in compound 4, the oil was dissolved in dry Et₂O (100 mL) under nitrogen. RED-Al (20 mL, 65% in toluene) was slowly added to the resulting solution at RT to control the evolution of hydrogen gas (H₂). The reaction was allowed to stir at RT overnight or when TLC showed the disappearance of the starting material (R_{f}=0.6 in 1:1 EtOAc:hexanes) and quenched slowly with cold MeOH or aqueous NH₄Cl to control the evolution of H₂. The resulting white suspension was filtered through a Celite pad and the filtrate was extracted with EtOAc (2×400 mL). Combined EtOAc extracts were dried over MgSO₄, filtered, and the filtrate evaporated to leave crude compound 5 and its syn diastereomer (not shown in FIG. 1) as a yellow oil (7.59 g).

Compound 6.

The oil containing compound 5 was dissolved in MeOH (200 mL), PTSA hydrate (0.63 g) was added, and the solution stirred at RT for 1 day and then at 50° C. for 2 days, at which point TLC suggested that all starting material (5) was gone. However, some polar material was present, suggesting that the acid had partially cleaved the BOC group. The reaction was worked up by adding saturated aqueous NH₄Cl (400 mL), and extracted with ether (3×300 mL). The combined ether phases were dried over Na₂SO₄, filtered, and the filtrate evaporated to dryness, leaving 5.14 g of oil. In order to re-protect whatever amine had formed, the crude product was dissolved in CH₂Cl₂ (150 mL), to which was added BOC₂O (2.44 g) and TEA (1.7 g). When TLC (1:1 hexanes/EtOAc) showed no more material remaining on the baseline, saturated aqueous NH₄Cl (200 mL) was added, and, after separating the organic phase, the mixture was extracted with CH₂Cl₂ (3×200 mL). Combined extracts were dried over Na₂SO₄, filtered, and the filtrated concentrated to dryness to yield a yellow oil (7.7 g) which was chromatographed on a silica column using a gradient of hexanes/EtOAc (up to 1:1) to separate the diastereomers. By TLC using 1:1 PE/EtOAc, the R_{f} for the anti isomer, compound 6, was 0.45. For the syn isomer (not shown in FIG. 1) the R_{f} was 0.40. The yield of compound 6 was 2.45 g (39% overall based on Garner's aldehyde). ¹H NMR of anti isomer (CDCl₃) δ 1.26 (br s, 20H), 1.32 (s, 9H), 1.45 (s, 9H), 1.56 (quintet, 2H, J=8 Hz), 2.06 (q, 2H, J=7 Hz), 2.52 (t, 2H, J=7 Hz), 2.55 (br s, 2H), 3.60 (br s, 1H), 3.72 (ddd, 1H, J=11.5 Hz, 7.0 Hz, 3.5 Hz), 3.94 (dt, 1H, J=11.5 Hz, 3.5 Hz), 4.32 (d, 1H, J=4.5 Hz), 5.28 (br s, 1H), 5.54 (dd, 1H, J=15.5 Hz, 6.5 Hz), 5.78 (dt, 1H, J=15.5 Hz, 6.5 Hz). ¹³C {¹H} NMR (CDCl₃) δ 156.95, 134.80, 129.66, 80.47, 75.46, 63.33, 56.17, 42.44, 32.98, 31.70, 30.58, 30.32, 30.31, 30.28, 30.20, 30.16, 30.00, 29.89, 29.80, 29.08, 29.03.

Anal. Calculated for C₂₇H₅₃NO₄S: C, 66.48; H, 10.95; N, 2.87. Found: C, 65.98; H, 10.46; N, 2.48.

Compound 7.

To a solution of the alcohol compound 6 (609.5 mg, 1.25 mmol) dissolved in dry pyridine (2 mL) was added CBr₄ (647.2 mg, 1.95 mmol, 1.56 equiv). The flask was cooled in an ice bath and P(OMe)₃ (284.7 mg, 2.29 mmol, 1.84 equiv) was added drop wise over 2 min. After 4 min. the ice bath was removed and after 12 hr. the mixture was diluted with ether (20 mL). The resulting mixture washed with aqueous HCl (10 mL, 2 N) to form an emulsion which separated on dilution with water (20 mL). The aqueous phase was extracted with ether (2×10 mL), then EtOAc (2×10 mL). The ether extracts and first EtOAc extract were combined and washed with aqueous HCl (10 mL, 2 N), water (10 mL), and saturated aqueous NaHCO₃ (10 mL). The last EtOAc extract was used to back-extract the aqueous washes. Combined organic phases were dried over MgSO₄, filtered, and the filtrate concentrated to leave crude product (1.16 g), which was purified by flash chromatography over silica (3×22 cm column) using CH₂Cl₂, then CH₂Cl₂-EtOAc (1:20, 1:6, 1:3, and 1:1-product started to elute, 6:4, 7:3). Early fractions contained 56.9 mg of oil. Later fractions provided product (compound 7, 476.6 mg, 64%) as clear, colorless oil.

Anal. Calculated for C₂₉H₅₈NO₇PS (595.82): C, 58.46; H, 9.81; N, 2.35. Found: C, 58.09; H, 9.69; N, 2.41.

Compound 8.

A flask containing compound 7 (333.0 mg, 0.559 mmol) and a stir bar was evacuated and filled with nitrogen. Acetonitrile (4 mL, distilled from CaH₂) was injected by syringe and the flask now containing a solution was cooled in an ice bath. Using a syringe, (CH₃)₃SiBr (438.7 mg, 2.87 mmol, 5.13 equiv.) was added over the course of 1 min. After 35 min., the upper part of the flask was rinsed with an additional portion of acetonitrile (1 mL) and the ice bath was removed. After another 80 min., an aliquot was removed, the solution dried by blowing nitrogen gas over it, and the residue analyzed by ¹H NMR in CDCl₃, which showed only traces of peaks ascribed to P—OCH₃ moieties. After 20 min., water (0.2 mL) was added to the reaction mixture, followed by the CDCl₃ solution used to analyze the aliquot, and the mixture was concentrated to ca. 0.5 mL volume on a rotary evaporator. Using acetone (3 mL) in portions the residue was transferred to a tared test tube, forming a pale brown solution. Water (3 mL) was added in portions. After addition of 0.3 mL, cloudiness was seen. After a total of 1 mL, a gummy precipitate had formed. As an additional 0.6 mL of water was added, more cloudiness and gum separated, but the final portion of water seemed not to change the appearance of the mixture. Overall, this process was accomplished over a period of several hours. The tube was centrifuged and the supernatant removed by pipet. The solid, no longer gummy, was dried over P₄O₁₀ in vacuo, leaving compound 8 (258.2 mg, 95%) as a monohydrate.

Anal. Calculated. for C₂₂H₄₆NO₅PS+H₂O (485.66): C, 54.40; H, 9.96; N, 2.88. Found: C, 54.59; H, 9.84; N, 2.95.

Compound 9.

Compound 8 (202.6 mg, 0.417 mmol) was added in a glove box to a test tube containing a stir bar, dry THF (3 mL) and glacial HOAc (3 mL). NBSCl (90 mg, 0.475 mmol, 1.14 equiv) were added, and after 0.5 hr., a clear solution was obtained. After a total of 9 hr., an aliquot was evaporated to dryness and the residue analyzed by ¹H NMR in CDCl₃. The peaks corresponding to CH₂StBu and CH₂SSAr suggested that reaction was about 75% complete, and comparison of the spectrum with that of pure NBSCl in CDCl₃ suggested that none of the reagent remained in the reaction. Therefore, an additional portion (24.7 mg, 0.130 mmol, 0.31 equiv) was added, followed 3 hr. later by an additional portion (19.5 mg, 0.103 mmol, 0.25 equiv). After another 1 hr., the mixture was transferred to a new test tube using THF (2 mL) to rinse and water (1 mL) was added.

Compound 10.

PMe₃ (82.4 mg, 1.08 mmol, 1.52 times the total amount of 2-nitrobenzenesulfenyl chloride added) was added to the clear solution compound 9 described above. The mixture grew warm and cloudy, with precipitate forming over time. After 4.5 hr., methanol was added, and the tube centrifuged. The precipitate settled with difficulty, occupying the bottom 1 cm of the tube. The clear yellow supernatant was removed using a pipet. Methanol (5 mL, deoxygenated with nitrogen) was added, the tube was centrifuged, and the supernatant removed by pipet. This cycle was repeated three times. When concentrated, the final methanol wash left only 4.4 mg of residue. The bulk solid residue was dried over P₄O₁₀ in vacuo, leaving compound 10 (118.2 mg, 68%) as a monohydrochloride.

Anal. Calculated for C₁₈H₃₈NO₅S+HCl (417.03): C, 51.84; H, 9.43; N, 3.36. Found: C, 52.11; H, 9.12; N, 3.30.

Compound 11.

Compound 6 (1.45 g, 2.97 mmol) was dissolved in AcOH (20 mL), and NBSCl (0.56 g, 2.97 mmol) was added all at once. The reaction was allowed to stir for 3 hr. or until the disappearance of the starting material and appearance of the product was observed by TLC [product R_{f}=0.65, starting material R_{f}=0.45, 1:1 EtOAc/hexanes]. The reaction was concentrated to dryness on a high vacuum line and the residue dissolved in THF/H₂O (100 mL of 10:1).

Compound 12.

Ph₃P (0.2.33 g, 8.91 mmol) was added all at once to the solution above that contained compound 11 and the reaction was allowed to stir for 3 hr. or until the starting material disappeared. The crude reaction mixture was concentrated to dryness on a high vacuum line, leaving a residue that contained compound 12.

Compound 13.

The residue above containing compound 12 was dissolved in DCM (50 mL) and TFA (10 mL). The mixture was stirred at RT for 5 hr. and concentrated to dryness. The residue was the loaded onto a column with silica gel and chromatographed with pure DCM, followed by DCM containing 5% MeOH, then 10% MeOH, to yield the final product, compound 13, as a sticky white solid (0.45 g, 46% yield from 5). ¹H NMR (CDCl₃) δ 1.27 (s), 1.33 (br m), 1.61 (p, 2H, J=7.5 Hz), 2.03 (br d, 2H, J=7 Hz), 2.53 (q, 2H, J=7.5 Hz), 3.34 (br s, 1H), 3.87 (br d, 2H, J=12 Hz), 4.48 (br s, 2H), 4.58 (br s, 2H), 5.42 (dd, 1H, J=15 Hz, 5.5 Hz), 5.82 (dt, 1H, J=15 Hz, 5.5 Hz), 7.91 (br s, 4H). ¹³C{¹H} NMR (CDCl₃) δ 136.85, 126.26, 57.08, 34.76, 32.95, 30.40, 30.36, 30.34, 30.25, 30.19, 30.05, 29.80, 29.62, 29.09, 25.34.

### Example 2: Synthetic Schemes for Making Thiolated Fatty Acids

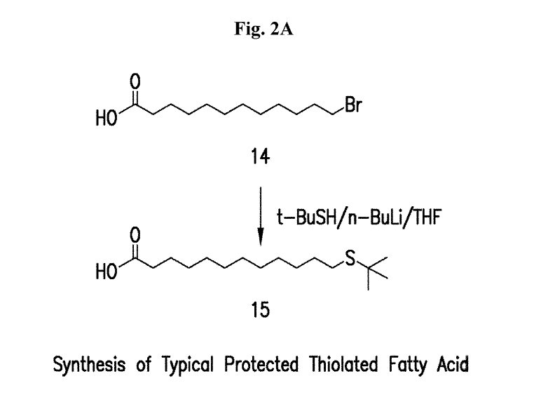

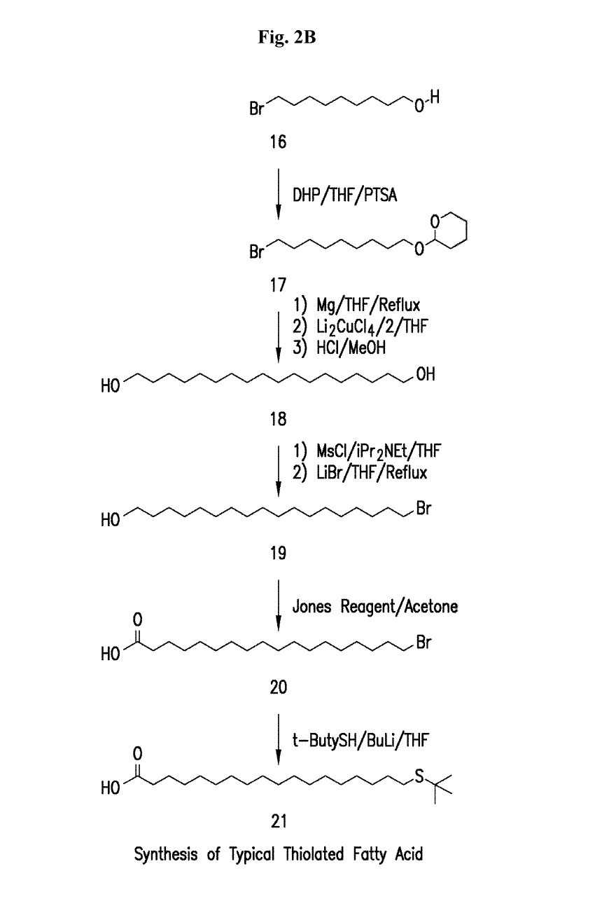

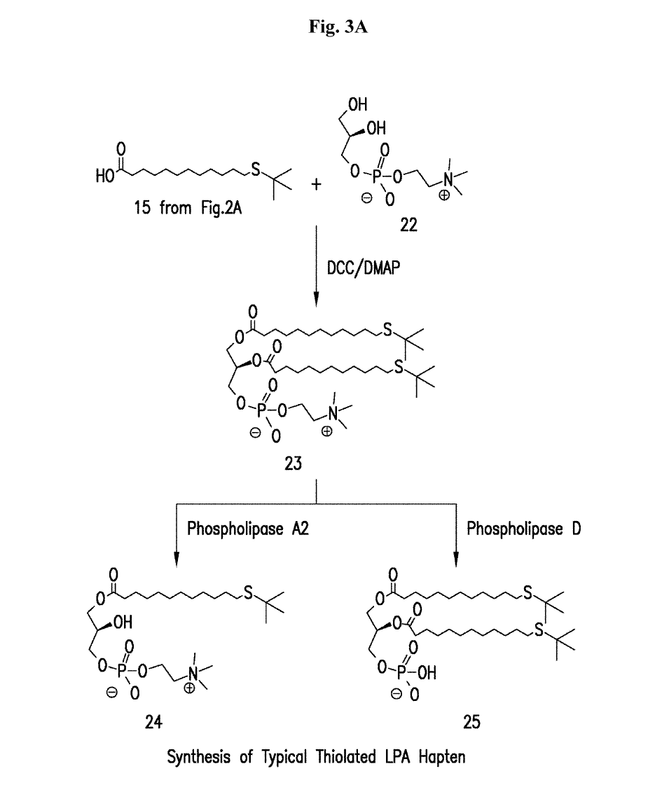

The synthetic approach described in this example details the preparation of a thiolated fatty acid to be incorporated into a more complex lipid structure that could be further complexed to a protein or other carrier and administered to an animal to elicit an immune response. The approach uses using conventional organic chemistry. A scheme showing the approach taken in this example is provided in FIG. 2, and the compound numbers in the synthetic description below refer to the numbered structures in FIG. 2.

Two syntheses are described. The first synthesis, for a C-12 thiolated fatty acid, starts with the commercially available 12-dodecanoic acid, compound 14. The bromine is then displaced with t-butyl thiol to yield the protected C-12 thiolated fatty acid, compound 15. The second synthesis, for a C-18 thiolated fatty acid, starts with the commercially available 9-bromo-nonanol (compound 16). The hydroxyl group in compound 16 is protected by addition of a dihydroyran group and the resulting compound, 17, is dimerized through activation of half of the brominated material via a Grignard reaction, followed by addition of the other half. The 18-hydroxy octadecanol (compound 18) produced following acid-catalyzed removal of the dihydropyran protecting group is selectively mono-brominated to form compound 19. During this reaction approximately half of the alcohol groups are activated for nucleophilic substitution by formation of a methane sulfonic acid ester. The alcohol is then oxidized to form the 18-bromocarboxylic acid, compound 20, which is then treated with t-butyl thiol to displace the bromine and form the protected, thiolated C-18 fatty acid, compound 21.

The protected thiolated fatty acids, each a t-butyl thioether, can be incorporated into a complex lipid and the protecting group removed using, e.g., one of the de-protecting approaches described in Examples 1 and 3. The resulting free thiol then can be used to complex with a protein or other carrier prior to inoculating animal with the hapten.

A. Synthesis of a C-12 Thiolated Fatty Acid

Compound 15.

t-Butyl thiol (12.93 g, 143 mmol) was added to a dry Schlenk flask, and Schlenk methods were used to put the system under nitrogen. Dry, degassed THF (250 mL) was added and the flask cooled in an ice bath. n-BuLi (55 mL of 2.5 M in hexanes, 137.5 mmol) was slowly added over 10 min by syringe. The mixture was allowed to stir at 0° C. for an hour. The bromoacid, compound 14 (10 g, 36 mmol), was added as a solid and the reaction heated and stirred at 60° C. for 24 hr. The reaction was quenched with 2 M HCl (250 mL), and extracted with ether (2×300 mL). The combined ethereal layers were dried with magnesium sulfate, filtered, and the filtrate concentrated by rotary evaporation to yield the thioether acid, compound 15 (10 g, 99% yield) as a beige powder. ¹H NMR (CDCl₃, 500 MHz) δ 1.25-1.35 (br s, 12H), 1.32 (s, 9H), 1.35-1.40 (m, 2H), 1.50-1.60 (m, 2H), 1.60-1.65 (m, 2H), 2.35 (t, 2H, J=7.5 Hz), 2.52 (t, 2H, J=7.5 Hz). Principal ion in HRMS (ES-TOF) was observed at m/z 311.2020, calculated for M+Na⁺ 311.2015.

B. Synthesis of a C-12 Thiolated Fatty Acid

Compound 17.

A dry Schlenk flask was charged with compound 16 (50 g, 224.2 mmol) and dissolved in dry degassed THF (250 mL) distilled from sodium/benzophenone. The flask was cooled in an ice bath and then PTSA (0.5 g, 2.6 mmol) was added. Dry, degassed DHP (36 g, 42.8 mmol) was then added slowly over 5 min. The mixture was allowed to warm up to RT and left to stir overnight and monitored by TLC (10:1 PE:EtOAc) until the reaction was deemed done by the complete disappearance of the spot for the bromoalcohol. TEA (1 g, 10 mmol) was then added to quench the PTSA. The mixture was then washed with cold sodium bicarbonate solution and extracted with EtOAc (3×250 mL). The organic layers were then dried with magnesium sulfate and concentrated to yield 68.2 g of crude product which was purified by column chromatography (10:1 PE:EtOAc) to yield 60 g (99% yield) of a colorless oil. ¹H NMR (CDCl₃, 500 MHz) δ 1.31 (br s, 6H), 1.41-1.44 (m, 2H), 1.51-1.62 (obscured multiplets, 6H), 1.69-1.74 (m, 1H), 1.855 (quintet, J=7.6 Hz, 2H), 3.41 (t, J=7 Hz, 2H), 3.48-3.52 (m, 2H), 3.73 (dt, 2H, J=6.5 Hz), 3.85-3.90 (m, 2H), 4.57 (t, 2H, J=3 Hz).

Compound 18.

Magnesium shavings (2.98 g, 125 mmol) were added to a flame-dried Schlenk flask along with a crystal of iodine. Dry THF (200 mL) distilled from sodium was then added and the system was degassed using Schlenk techniques. Compound 17 (30 g, 97 mmol) was then slowly added to the magnesium over 10 min. and the solution was placed in an oil bath at 65° C. and allowed to stir overnight. The reaction was deemed complete by TLC by quenching an aliquot with acetone and observing the change in RF in a 10:1 PE:EtOAc mixture. The Grignard solution was then transferred by cannula to a three-necked flask under nitrogen containing additional compound 17 (30 g, 97 mmol). The flask containing the resulting mixture was then cooled to 0° C. in an ice bath and a solution of Li₂CuCl₄ (3 mL of 1 M) was then added via syringe. The reaction mixture turned a very dark blue within a few minutes. This mixture was left to stir overnight. The next morning the reaction was deemed complete by TLC (10:1 PE:EtOAc), quenched with a saturated NH₄Cl solution, and then extracted into ether (3×250 mL). The ether layers were dried with magnesium sulfate and concentrated to yield crude product (40 g), which was dissolved in MeOH. Concentrated HCl (0.5 mL) was then added, which resulted in the formation of a white emulsion, which was left to stir for 3 hr. The white emulsion was then filtered to yield 16 g (58% yield) of the pure diol, compound 18. ¹H NMR (CDCl₃, 200 MHz) δ 1.26 (br s, 24H), 1.41-1.42 (m, 4H), 1.51-1.68 (m, 4H), 3.65 (t, 4H, J=6.5 Hz).

Compound 19.

The symmetrical diol, compound 18 (11 g, 38.5 mmol), was added to a dry Schlenk flask under nitrogen, then dry THF (700 mL) distilled from sodium was added. The system was degassed and the flask put in an ice bath. Diisopropylethylamine (6.82 mL, 42.3 mmol) was added via syringe, followed by MSCl (3.96 g, 34.4 mmol) added slowly, and the mixture was left to stir for 1 hr. The reaction was quenched with saturated NaH₂PO₄ solution (300 mL), and then extracted with EtOAc (3×300 mL). The organic layers were then combined, dried with MgSO₄, and concentrated to yield 14 g of a mixture of the diol, monomesylate, and dimesylate. NMR showed a 1:0.8 mixture of CH₂OH:CH₂OMs protons. The mixture was then dissolved in dry THF (500 mL), deoxygenated, and to it was added LiBr (3.5 g, 40.23 mmol). This mixture was allowed reflux overnight, upon which the reaction was quenched with water (150 mL), and extracted with EtOAc (3×250 mL). The organic layer was then dried with MgSO₄, and concentrated to yield a mixture of brominated products that were then purified by flash chromatography (DCM) to yield compound 19 (3.1 g, 25% yield) as a white powder. ¹H NMR (CDCl₃, 500 MHz) δ 1.26 (br s, 26H), 1.38-1.46 (m, 2H), 1.55 (quintet, 2H, J=7.5 Hz), 1.85 (quintet, 2H, J=7.5 Hz), 3.403 (t, 2H, J=6.8 Hz), 3.66 (t, 2H, J=6.8 Hz).

Compound 20.

A round bottom flask was charged with compound 19 (2.01 g, 5.73 mmol) and the solid dissolved in reagent grade acetone (150 mL). Simultaneously, Jones reagent was prepared by dissolving CrO₃ (2.25 g, 22 mmol) in H₂SO₄ (4 mL) and then slowly adding 10 mL of cold water and letting the solution stir for 10 min. The cold Jones reagent was then added to the round bottom flask slowly over 5 min., after which the solution stirred for 1 hr. The resulting orange solution turned green within several minutes. The mixture was then quenched with water (150 mL) extracted twice in ether (3×150 mL). The ether layers were then dried with magnesium sulfate, and concentrated to yield compound 20 (2.08 g, 98% yield) as a white powder. ¹H NMR (CDCl₃, 200 MHz) δ 1.27 (br s, 26H), 1.58-1.71 (m, 2H), 1.77-1.97 (m, 2H), 2.36 (t, 2H, J=7.4 Hz), 3.42 (t, 2H, J=7 Hz).

Compound 21.

t-Butylthiol (11.32 g, 125 mmol) was added to a dry Schlenk flask and dissolved in dry THF (450 mL) distilled from sodium. The solution was deoxygenated by bubbling nitrogen through it before the flask was placed in an ice bath. n-BuLi solution in hexanes (70 mL of 1.6 M) was then added slowly via syringe over 10 min. This mixture was allowed to stir for 1 hr., then compound 20 (5.5 g, 16.2 mmol) was added and the solution was left to reflux at 60° C. overnight. The next morning an aliquot was worked up, analyzed by NMR, and the reaction deemed complete. The reaction was quenched with HCl (200 mL of 2 M) and extracted with ether (3×250 mL). The ethereal layers were then dried with magnesium sulfate, filtered, and the filtrate concentrated to yield the product, compound 21, as a white solid (5 g, 90% yield). ¹H NMR (CDCl₃, 200 MHz) δ 1.26 (br s, 26H), 1.32 (br s, 9H), 1.48-1.70 (m, 4H), 2.35 (t, 2H, J=7.3 Hz), 2.52 (t, 2H, J=7.3 Hz). ¹³C NMR (CDCl₃, 200 MHz) δ 24.69, 28.35, 29.05, 29.21, 29.28, 29.39, 29.55, 29.89, 31.02 (3C), 33.98, 41.75, 179.60.

### Example 3: Synthetic Scheme for Making a Thiolated Analog of LPA

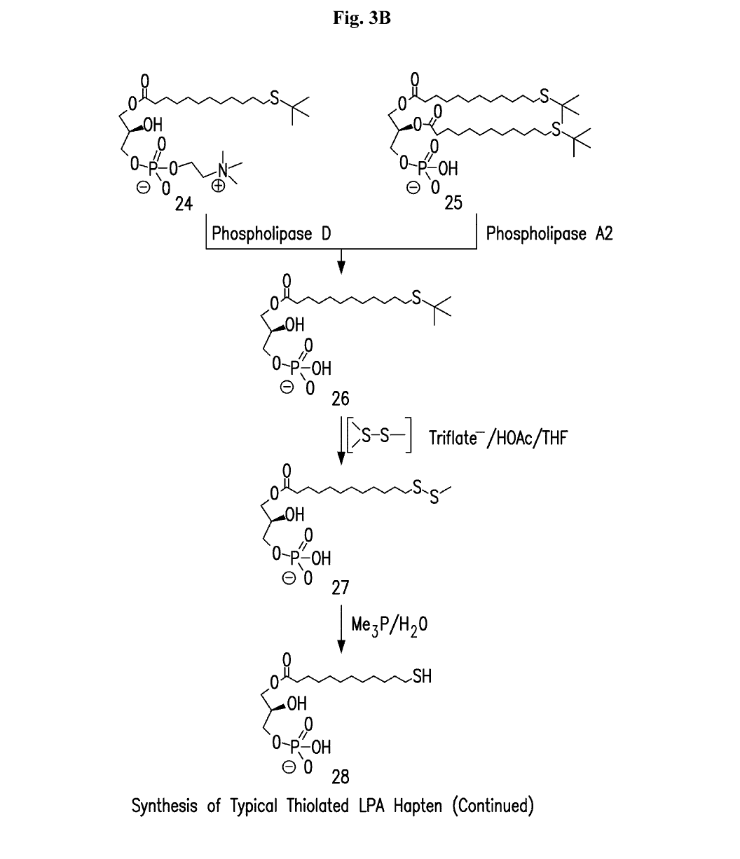

The synthetic approach described in this example results in the preparation of thiolated LPA. The LPA analog can then be further complexed to a carrier, for example, a protein carrier, which can then be administered to an animal to elicit an immugenic response to LPA. This approach uses both organic chemistry and enzymatic reactions, the synthetic scheme for which is provided in FIG. 3. The compound numbers in the synthetic description below refer to the numbered structures in FIG. 3.

The starting materials were compound 15 in Example 2 and enantiomerically pure glycerophosphocholine (compound 22). These two chemicals combined to yield the di-acetylated product, compound 23, using DCC to facilitate the esterification. In one synthetic process variant, the resulting di-acylated glycerophosphocholine was treated first with phospholipase-A2 to remove the fatty acid at the sn-2 position of the glycerol backbone to produce compound 24. This substance was further treated with another enzyme, phospholipase-D, to remove the choline and form compound 26. In another synthetic process variant, the phospholipase-D treatment preceded the phospholipase-A2 treatment to yield compound 25, and treatment of compound 25 with phospholipase-D then yields compound 26. Both variants lead to the same product, the phosphatidic acid derivative, compound 26. The t-butyl protecting group in compound 26 is then removed, first using trimethyl disulfide triflate to produce compound 27, followed by a disulfide reduction to produce the desired LPA derivative, compound 28. As those in the art will appreciate, the nitrobenzyl sulfenyl reaction sequence described in Example 1 can also be used to produce compound 28.

Compound 23.

To a flame-dried Schlenk flask were added the thioether acid, compound 15 (10 g, 35.8 mmol), compound 22 (glycerophosphocholine-CdCl2 complex, 4.25 g, 8.9 mmol), DCC (7.32 g, 35.8 mmol), and DMAP (2.18 g, 17.8 mmol), after which the flask was evacuated and filled with nitrogen. A minimal amount of dry, degassed DCM was added (100 mL), resulting in a cloudy mixture. The flask was covered with foil and then left to stir until completed, as by TLC (silica, 10:5:1 DCM:MeOH:concentrated NH4OH). The insolubility of compound 16 precluded monitoring its disappearance by TLC, but the reaction was stopped when the product spot of Rf 0.1 was judged not to be increasing in intensity. This typically required 3 to 4 days, and in some cases, addition of more DCC and DMAP. Upon completion, the reaction mixture was filtered, and the filtrate concentrated to yield a yellow oil, which was purified using flash chromatography using the solvent system described above to yield 3.6 g (50% yield) of a clear wax containing a mixture of compound 23 and monoacylated products in a ratio of 5 to 1, as estimated from comparing the integrals for the peaks for the (CH3)3N—, —CH2StBu and —CH2COO— moieties. Analysis of the oil by HRMS (ESI-TOF) produced a prominent ion at m/z 820.4972, calculated for M+Na⁺=C₄₀H₈₀NNaO₈PS₂_{⁺}820.4960.

A. Synthesis Variant 1—Phospholipase-A2 Treatment

Compound 24.

A mixture of compound 23 and monoacetylated products as described above (3.1 g, 3.9 mmol) was dissolved in Et₂O (400 mL) and methanol (30 mL). Borate buffer (100 mL, pH 7.4 0.1M, 0.072 mM in CaCl2) was added, followed by phospholipase-A2 (from bee venom, 130 units, Sigma). The resulting mixture was left to stir for 10 hr., at which point TLC (silica, MeOH:water 4:1—the previous solvent system 10:5:1 DCM:MeOH:concentrated NH4OH proved ineffective) showed the absence of the starting material (Rf=0.7) and the appearance of a new spot (Rf=0.2). The organic and aqueous layers were separated and the aqueous layer was washed with ether (2×250 mL). The product was extracted from the aqueous layer with a mixture of DCM:MeOH (2:1, 2×50 mL). The organic layers were then concentrated by rotary evaporation to yield product as a white wax (1.9 g, 86% yield) that NMR showed to be a pure product (compound 24). ¹H NMR (CDCl3, 500 MHz) δ 1.25-1.27 (br s, 12H), 1.31 (s, 9H), 1.35-1.45 (m, 2H), 1.52-1.60 (m, 4H), 2.31 (t, 2H, J=7.5 Hz), 2.51 (t, 2H, J=7.5 Hz), 3.28 (br s, 9H) 3.25-3.33 (br s, 2H), 3.78-3.86 (m, 1H), 3.88- 3.96 (m, 2H), 4.04-4.10 (m, 2H), 4.26-4.34 (m, 2H). Analysis of the wax by HRMS (ESI-TOF) produced a prominent ion at m/z 550.2936, calculated for M+Na⁺ 550.2943 (C₂₄H₅₀NNaO₇PS₂_{⁺}), and an m/z at 528.3115, calculated for MK⁺ 528.3124 (C₂₄H₅₁NO₇PS₂_{⁺}).

Anal. Calculated. for C24H50NO7PS+2 H2O (563.73): C, 51.13; H, 9.66; N, 2.48. Found: C, 50.90; H, 9.37; N, 2.76.

Compound 26.

The lyso compound 24 (1.5 g, 2.7 mmol) was dissolved in a mixture of sec-butanol (5 mL) and Et₂O (200 mL), and the resulting cloudy mixture was sonicated until the cloudiness dissipated. Buffer (200 mL, pH 5.8, 0.2 M NaOAc, 0.08 M CaCl2) was added, followed by cabbage extract (80 mL of extract from savoy cabbage (which contains phospholipase-D), containing 9 mg of protein/mL). The reaction was stirred for 1 day and monitored by TLC (C18 RP SiO2, 5:1 ACN:water), Rf of starting material and product=0.3 and 0.05, respectively. In order to push the reaction to completion, as needed an additional portion of cabbage extract (50 mL) was added and the reaction stirred a further day. This process was repeated twice more, as needed to complete the conversion. When the reaction was complete, the mixture was concentrated on the rotary evaporator to remove the ether, and then EDTA solution (0.5 M, 25 mL) was added and the product extracted into a 5:4 mixture of MeOH:DCM (300 mL). Concentration of the organic layer followed by recrystallization of the residue from DCM and acetone afforded pure product (0.9 g, 75% yield). ¹H NMR (CDCl3, 200 MHz) δ 1.25-1.27 (br s, 12H), 1.33 (s, 9H), 1.52-1.60 (m, 4H), 2.34 (t, 2H, J=7.5 Hz), 2.52 (t, 2H, J=7.5 Hz), 3.6-3.8 (br s, 1H), 3.85-3.97 (br s, 2H), 4.02-4.18 (m, 2H).

Compound 27.

The protected sample LPA, compound 26 (0.150 g, 0.34 mmol), was methanol washed and added to a vial in the glove box. This was then suspended in a mixture of AcOH:THF (1:1, 10 mL), which never fully dissolved even after 1 hr. of sonication. Solid [Me₂SSMe]OTf (0.114 g, 0.44 mmol) was then added. This was left to stir for 18 hr. The reaction was monitored by removing an aliquot, concentrating it to dryness under vacuum, and re-dissolving or suspending the residue in CD₃OD for observing the ¹H NMR shift of the CH₂ peak closest to the sulfur. The starting material had a peak at 2.52 ppm, whereas the unsymmetrical disulfide formed at this juncture had a peak at around 2.7 ppm. This material (compound 27) was not further isolated or characterized.

Compound 28.

The mixture containing compound 27 was treated with water (100 μL) immediately followed by PMe3 (0.11 g, 1.4 mmol). After stirring for 3 hr. the solvent was removed by vacuum to yield an insoluble white solid. Methanol (5 mL) was added, the mixture centrifuged, and the mother liquor decanted. Vacuum concentration yielded 120 mg (91% yield) of compound 28, a beige solid. Compound 28 is a thiolated LPA hapten that can be conjugated to a carrier, for example, albumin or KLH, via disulfide bond formation. Characterization of compound 28: ¹H NMR (1:1 CD₃OD:CD₃CO₂D, 500 MHz) δ 1.25-1.35 (br s, 12H), 1.32-1.4 (m, 2H), 1.55-1.6 (m, 4H), 2.34 (t, 2H, J=7), 2.47 (t, 2H, J=8.5), 3.89-3.97 (br s, 2H), 3.98-4.15 (m, 2H), 4.21 (m, 1H). Negative ion ES of the sample dissolved in methanol produced a predominant ion at m/z=385.1.

### Example 4: Monoclonal Antibodies to LPA

Antibody Production

Using an approach employing a derivatized lipid as described in previous examples, a C-12 thio-LPA analog (compound 28 in Example 3) as the key component of a hapten formed by the cross-linking of the analog via the reactive SH group to a protein carrier (KLH) via standard chemical cross-linking using either IOA or SMCC as the cross-linking agent, monoclonal antibodies against LPA were generated. To do this, mice were immunized with the thio-LPA-KLH hapten (in this case, thiolated-LPA:SMCC:KLH). Of the 80 mice immunized against the LPA analog, the five animals that showed the highest titers against LPA (determined using an ELISA in which the same LPA analog (compound 28) as used in the hapten was conjugated to BSA using SMCC and laid down on the ELISA plates) were chosen for moving to the hybridoma phase of development.

The spleens from these five mice were harvested and hybridomas were generated by standard techniques. Briefly, one mouse yielded hybridoma cell lines (designated 504A). Of all the plated hybridomas of the 504A series, 66 showed positive antibody production as measured by the previously-described screening ELISA.

Table 1, below, shows the antibody titers in cell supernatants of hybridomas created from the spleens of two of mice that responded to an LPA analog hapten in which the thiolated LPA analog was cross-linked to KLH using heterobifunctional cross-linking agents. These data demonstrate that the anti-LPA antibodies do not react either to the crosslinker or to the protein carrier. Importantly, the data show that the hybridomas produce antibodies against LPA, and not against S1P.

TABLE 1
LPA hybridomas

| | | | LPA | S1P | |
|---|---|---|---|---|---|
| | 3rd bleed | | binding | binding | Cross |
| mouse | titer OD at | Supernatants | OD at | OD at | reactivity |
| # | 1:312,500 | from 24 well | 1:20 | 1:20 | w/S1P* |
| 1 | 1.242 | 1.A.63 | 1.197 | 0.231 | low |
| | | 1.A.65 | 1.545 | 0.176 | none |
| 2 | 0.709 | 2.B.7 | 2.357 | 0.302 | low |
| | | 2.B.63 | 2.302 | 0.229 | low |
| | | 2.B.83 | 2.712 | 0.175 | none |
| | | 2.B.104 | 2.57 | 0.164 | none |
| | | 2.B.IB7 | 2.387 | 0.163 | none |
| | | 2.B.3A6 | 2.227 | 0.134 | none |

*Cross reactivity with S1P from 24 well supernatants: high = OD >1.0-2.0 at [1:20]; mid = OD 0.4-1.0 at [1:20]; low = OD 0.4-0.2 at [1:20]; none = OD <0.2 OD at [1:20].

The development of anti-LPA mAbs in mice was monitored by ELISA (direct binding to 12:0 and 18:1 LPA and competition ELISA). A significant immunological response was observed in at least half of the immunized mice and five mice with the highest antibody titer were selected to initiate hybridoma cell line development following spleen fusion.

After the initial screening of over 2000 hybridoma cell lines generated from these 5 fusions, a total of 29 anti-LPA secreting hybridoma cell lines exhibited high binding to 18:1 LPA. Of these hybridoma cell lines, 24 were further subcloned and characterized in a panel of ELISA assays. From the 24 clones that remained positive, six hybridoma clones were selected for further characterization. Their selection was based on their superior biochemical and biological properties. Mouse hybridoma cell lines 504B-6C2, 504B7.1, 504B58/3F8, 504A63.1 and 504B3A6 (corresponding to clones referred to herein as B3, B7, B58, A63, and B3A6, respectively) were received on May 8, 2007 by the American Type Culture Collection (ATCC Patent Depository, 10801 University Blvd., Manassas, Va. 20110) for patent deposit purposes on behalf of LPath Inc. and were granted deposit numbers PTA-8417, PTA-8420, PTA-8418, PTA-8419 and PTA-8416, respectively.

All anti-LPA antibodies and portions thereof referred to herein were derived from these cell lines.

Direct Binding Kinetics

The binding of 6 anti-LPA mAbs (B3, B7, B58, A63, B3A6, D22) to 12:0 and 18:0 LPA (0.1 uM) was measured by ELISA. EC₅₀ values were calculated from titration curves using 6 increasing concentrations of purified mAbs (0 to 0.4 ug/ml). EC₅₀ represents the effective antibody concentration with 50% of the maximum binding. Max denotes the maximal binding (expressed as OD450). Results are shown in Table 2.

TABLE 2
Direct Binding Kinetics of Anti-LPA mAbs

| | | B3 | B7 | B58 | D22 | A63 | B3A6 |
|---|---|---|---|---|---|---|---|
| 12:0 LPA | EC₅₀ (nM) | 1.420 | 0.413 | 0.554 | 1.307 | 0.280 | 0.344 |
| | Max (OD450) | 1.809 | 1.395 | 1.352 | 0.449 | 1.269 | 1.316 |
| 18:1 LPA | EC₅₀ (nM) | 1.067 | 0.274 | 0.245 | 0.176 | 0.298 | 0.469 |
| | Max (OD450) | 1.264 | 0.973 | 0.847 | 0.353 | 1.302 | 1.027 |

The kinetics parameters kₐ (association rate constant), k_{d} (disassociation rate constant) and K_{D} (association equilibrium constant) were determined for the 6 lead candidates using the BIAcore 3000 Biosensor machine. In this study, LPA was immobilized on the sensor surface and the anti-LPA mAbs were flowed in solution across the surface. As shown, all six mAbs bound LPA with similar K_{D} values ranging from 0.34 to 3.8 pM and similar kinetic parameters.

The Anti-LPA Murine mAbs Exhibit High Affinity to LPA

LPA was immobilized to the sensor chip at densities ranging 150 resonance units. Dilutions of each mAb were passed over the immobilized LPA and kinetic constants were obtained by nonlinear regression of association/dissociation phases. Errors are given as the standard deviation using at least three determinations in duplicate runs. Results are shown in Table 3. Apparent affinities were determined by K_{D}=kₐ/k_{d}.

kₐ=Association rate constant in M⁻¹s⁻¹k_{d}=Dissociation rate constant in s⁻¹

TABLE 3
Affinity of anti-LPA mAb for LPA

| mAbs | kₐ (M⁻¹ s⁻¹) | k_{d} (s⁻¹) | K_{D} (pM) |
|---|---|---|---|
| A63 | 4.4 ± 1.0 × 10⁵ | 1 × 10⁻⁶ | 2.3 ± 0.5 |
| B3 | 7.0 ± 1.5 × 10⁵ | 1 × 10⁻⁶ | 1.4 ± 0.3 |
| B7 | 6.2 ± 0.1 × 10⁵ | 1 × 10⁻⁶ | 1.6 ± 0.1 |
| D22 | 3.0 ± 0.9 × 10⁴ | 1 × 10⁻⁶ | 33 ± 10 |
| B3A6 | 1.2 ± 0.9 × 10⁶ | 1.9 ± 0.4 × 10⁻⁵ | 16 ± 1.2 |

Specificity Profile of Six Anti-LPA mAbs.

Many isoforms of LPA have been identified to be biologically active and it is preferable that the mAb recognize all of them to some extent to be of therapeutic relevance. The specificity of the anti-LPA mAbs was evaluated utilizing a competition assay in which the competitor lipid was added to the antibody-immobilized lipid mixture.

Competition ELISA assays were performed with the anti-LPA mAbs to assess their specificity. Thiolated 18:1 LPA-BSA conjugate was captured on ELISA plates. Each competitor lipid (up to 10 uM) was serially diluted in BSA (1 mg/ml)-PBS and then incubated with the mAbs (3 nM). Mixtures were then transferred to LPA coated wells and the amount of bound antibody was measured with a secondary antibody. Data are normalized to maximum signal (A₄₅₀) and are expressed as percent inhibition. Assays were performed in triplicate. IC₅₀: Half maximum inhibition concentration; MI: Maximum inhibition (% of binding in the absence of inhibitor); ---: not estimated because of weak inhibition. A high inhibition result indicates recognition of the competitor lipid by the antibody. As shown in Table 4, all the anti-LPA mAbs recognized the different LPA isoforms.

TABLE 4
Specificity profile of anti-LPA mAbs.

| | 14:0 LPA | | 16:0 LPA | | 18:1 LPA | | 18:2 LPA | | 20:4 LPA | |
|---|---|---|---|---|---|---|---|---|---|---|
| | IC₅₀ | MI | IC₅₀ | MI | IC₅₀ | MI | IC₅₀ | MI | IC₅₀ | MI |
| | uM | % | uM | % | uM | % | uM | % | uM | % |
| B3 | 0.02 | 72.3 | 0.05 | 70.3 | 0.287 | 83 | 0.064 | 72.5 | 0.02 | 67.1 |
| B7 | 0.105 | 61.3 | 0.483 | 62.9 | >2.0 | 100 | 1.487 | 100 | 0.161 | 67 |
| B58 | 0.26 | 63.9 | 5.698 | >100 | 1.5 | 79.3 | 1.240 | 92.6 | 0.304 | 79.8 |
| B104 | 0.32 | 23.1 | 1.557 | 26.5 | 28.648 | >100 | 1.591 | 36 | 0.32 | 20.1 |
| D22 | 0.164 | 34.9 | 0.543 | 31 | 1.489 | 47.7 | 0.331 | 31.4 | 0.164 | 29.5 |
| A63 | 1.147 | 31.9 | 5.994 | 45.7 | — | — | — | — | 0.119 | 14.5 |
| B3A6 | 0.108 | 59.9 | 1.151 | 81.1 | 1.897 | 87.6 | — | — | 0.131 | 44.9 |

Interestingly, the anti-LPA mAbs were able to discriminate between 12:0 (lauroyl), 14:0 (myristoyl), 16:0 (palmitoyl), 18:1 (oleoyl), 18:2 (linoleoyl) and 20:4 (arachidonoyl) LPAs. A desirable EC₅₀ rank order for ultimate drug development is 18:2>18:1>20:4 for unsaturated lipids and 14:0>16:0>18:0 for the saturated lipids, along with high specificity. The specificity of the anti-LPA mAbs was assessed for their binding to LPA related biolipids such as distearoyl-phosphatidic acid, lysophosphatidylcholine, S1P, ceramide and ceramide-1-phosphate. None of the antibodies demonstrated cross-reactivity to distearoyl PA and LPC, the immediate metabolic precursor of LPA.

### Example 5: Cloning of the Murine Anti-LPA Antibodies—Overview

Chimeric antibodies to LPA were generated using the variable domains (Fv) containing the active LPA binding regions of one of three murine antibodies from hybridomas with the Fc region of a human IgG1 immunoglobulin. The Fc regions contained the CH1, CH2, and CH3 domains of the human antibody. Without being limited to a particular method, chimeric antibodies could also have been generated from Fc regions of human IgG1, IgG2, IgG3, IgG4, IgA, or IgM. As those in the art will appreciate, “humanized” antibodies can be generated by grafting the complementarity determining regions (CDRs, e.g., CDR1-4) of the murine anti-LPA mAbs with a human antibody framework regions (e.g., Fr1, Fr4, etc.) such as the framework regions of an IgG1.

The overall strategy for cloning of the murine mAb against LPA consisted of cloning the murine variable domains of both the light chain (VL) and the heavy chain (VH) from each antibody. The consensus sequences of the genes show that the constant region fragment is consistent with a gamma isotype and that the light chain is consistent with a kappa isotype. The murine variable domains were cloned together with the constant domain of the human antibody light chain (CL) and with the constant domain of the human heavy chain (CH1, CH2, and CH3), resulting in a chimeric antibody construct.

The variable domains of the light chain and the heavy chain were amplified by PCR. The amplified fragments were cloned into an intermediate vector (pTOPO). After verification of the sequences, the variable domains were then assembled together with their respective constant domains. The variable domain of the light chain was cloned into pCONkappa2 and the variable domain of the heavy chain was cloned into pCONgamma1f. The cloning procedure included the design of an upstream primer to include a signal peptide sequence, a consensus Kozak sequence preceding the ATG start codon to enhance translation initiation, and the 5′ cut site, HindIII. The downstream primer was designed to include the 3′ cut site Apal for the heavy chain and BsiWl for the light chain.

The vectors containing the variable domains together with their respective constant domains were transfected into mammalian cells. Three days after transfections, supernatants were collected and analyzed by ELISA for binding to LPA. Detailed methods for cloning, expression and characterization of the anti-LPA antibody variable domains are shown in US Patent Application Publication Nos: 20090136483, commonly owned with the instant invention and incorporated herein by reference in its entirety.

Binding characteristics for the chimeric antibodies are shown in Table 5. “HC” and “LC” indicate the identities of the heavy chain and light chain, respectively.

TABLE 5
Binding characteristics of the chimeric
anti-LPA antibodies B3, B7, and B58.

| | | | | | |
|---|---|---|---|---|---|
| | | | Titer | EC50 | Max |
| HC | x | LC | (ug/ml) | (ng/ml) | OD |

| | | | | | |
|---|---|---|---|---|---|
| 1 | B7 | B7 | 3.54 | 43.24 | 2.237 |
| 2 | B7 | B58 | 1.84 | 25.79 | 1.998 |
| 3 | B7 | B3 | 2.58 | 24.44 | 2.234 |
| 4 | B58 | B7 | 3.80 | 38.99 | 2.099 |
| 5 | B58 | B58 | 3.42 | 41.3 | 2.531 |
| 6 | B58 | B3 | 2.87 | 29.7 | 2.399 |
| 7 | B3 | B7 | 4.18 | 49.84 | 2.339 |
| 8 | B3 | B58 | 0.80 | 20.27 | 2.282 |
| 9 | B3 | B3 | 4.65 | 42.53 | 2.402 |

It can be seen from Table 5 that it is possible to optimize antibody binding to LPA by recombining light chains and heavy chains from different hybridomas (i.e., different clones) into chimeric molecules.

### Example 6 Lpath's Lead Murine Antibody, Lpathomab™ (LT3000)—Overview

Murine antibody clone B7 was chosen as the lead compound and renamed Lpathomab™, also known as LT3000. As described above, this murine anti-LPA mAb, was derived from a hybridoma cell line following immunization of mice with a protein-derivatized LPA immunogen. A hybridoma cell line with favorable properties was identified and used to produce a monoclonal antibody using standard hybridoma culture techniques.

Applicant has performed a comprehensive series of pre-clinical efficacy studies to confirm the potential therapeutic utility of an anti-LPA-antibody-based approach. It is believed that antibody neutralization (e.g., reduction in effective concentration) of extracellular LPA could result in a marked decrease in disease progression in humans. For cancer, LPA neutralization could result in inhibition of tumor proliferation and the growing vasculature needed to support tumor growth. Furthermore, recent research suggests that many angiogenesis inhibitors may also act as anti-invasive and anti-metastatic compounds that could also mitigate the spread of cancer to sites distant from the initial tumor. For fibrosis, LPA neutralization could result in a reduction of the inflammation and fibrosis associated with the aberrant wound-healing response following tissue injury. Thus, Lpathomab™ could have several mechanisms of action, including:
A direct effect on tumor cell growth, migration and susceptibility to chemotherapeutic agents
An indirect effect on tumors through anti-angiogenic effects
An additional indirect effect on tumors by preventing the release and neutralization of synergistic pro-angiogenic growth factors
A direct effect on proliferation, migration, and transformation of fibroblasts to the myofibroblast phenotype and collagen production by myofibroblasts
An indirect effect on tissue fibrosis by preventing the expression and release of synergistic pro-angiogenic, pro-inflammatory and pro-fibrotic growth factors

### Example 7: Biophysical Properties of Lpathomab/LT3000

Lpathomab/LT3000 has high affinity for the signaling lipid LPA (K_{D} of 1-50 pM as demonstrated by surface plasmon resonance in the BiaCore assay, and in a direct binding ELISA assay); in addition, LT3000 demonstrates high specificity for LPA, having shown no binding affinity for over 100 different bioactive lipids and proteins, including over 20 bioactive lipids, some of which are structurally similar to LPA. The murine antibody is a full-length IgG1k isotype antibody composed of two identical light chains and two identical heavy chains with a total molecular weight of 155.5 kDa. The biophysical properties are summarized in Table 6.

TABLE 6
General Properties of Lpathomab (LT3000)

| | |
|---|---|
| Identity | LT3000 |
| Antibody isotype | Murine IgG1k |
| Specificity | Lysophosphatidic acid (LPA) |
| Molecular weight | 155.5 kDa |
| OD of 1 mg/mL | 1.35 (solution at 280 nm) |
| K_{D} | 1-50 pM |
| Apparent Tm | 67° C. at pH 7.4 |
| Appearance | Clear if dissolved in 1x PBS buffer (6.6 mM |
| | phosphate, 154 mM sodium chloride, pH 7.4) |
| Solubility | >40 mg/mL in 6.6 mM phosphate, 154 mM |
| | sodium chloride, pH 7.4 |

Lpathomab has also shown biological activity in preliminary cell based assays such as cytokine release, migration and invasion; these are summarized in Table 7 along with data showing specificity of LT3000 for LPA isoforms and other bioactive lipids, and in vitro biological effects of LT3000.

TABLE 7
LT3000 (Lpathomab, B7 antibody)

| | A. Competitor Lipid | | | | |
|---|---|---|---|---|---|
| | 14:0 | 16:0 | 18:1 | 18:2 | 20:4 |
| | LPA | LPA | LPA | LPA | LPA |
| IC₅₀ (□M) | 0.105 | 0.483 | >2.0 | 1.487 | 0.161 |
| MI (%) | 61.3 | 62.9 | 100 | 100 | 67 |

| B. Competitor Lipid | LPC | S1P | C1P | Cer | DSPA |
|---|---|---|---|---|---|
| MI (%) | 0 | 2.7 | 1.0 | 1 | 0 |

| C. Cell based assay | LPA isoform | |
|---|---|---|
| | | % Inhibition |
| | | (over LPA taken as 100) |
| Migration | 18:1 | 35* |
| Invasion | 14:0 | 95* |
| IL-8 Release | 18:1 | 20 |
| IL-6 Release | 18:1 | 23* |
| | | % Induction |
| | | (over LPA + TAXOL taken as 100) |
| Apoptosis | 18:1 | 79 |

A. Competition ELISA assay was performed with Lpathomab and 5 LPA isoforms. 18:1 LPA was captured on ELISA plates. Each competitor lipid (up to 10□M) was serially diluted in BSA/PBS and incubated with 3 nM Lpathomab. Mixtures were then transferred to LPA coated wells and the amount of bound antibody was measured.
B. Competition ELISA was performed to assess specificity of Lpathomab. Data were normalized to maximum signal (A₄₅₀) and were expressed as percent inhibition (n = 3). IC₅₀: half maximum inhibition concentration; MI %: maximum inhibition (% of binding in the absence of inhibitor).
C. Migration assay: Lpathomab (150□g/mL) reduced SKOV3 cell migration triggered by 1□M LPA (n = 3); Invasion assay: Lpathomab (15 mg/mL) blocked SKOV3 cell invasion triggered by 2□M LPA (n = 2); Cytokine release of human IL-8 and IL-6: Lpathomab (300-600 □g/mL, respectively) reduced 1□M LPA-induced release of pro-angiogenic and metastatic IL-8 and IL-6 in SKOV3 conditioned media (n = 3). Apoptosis: SKOV3 cells were treated with 1□M Taxol; 1□M LPA blocked Taxol induced caspase-3 activation. The addition to Lpathomab (150□g/mL) blocked LPA-induced protection from apoptosis (n = 1). Data Analysis: Student-t test,
*denotes p < 0.05.

The potent and specific binding of Lpathomab/LT3000 to LPA results in reduced availability of extracellular LPA with potentially therapeutic effects against cancer-, angiogenic- and fibrotic-related disorders.

A second murine anti-LPA antibody, B3, was also subjected to binding analysis as shown in Table 8.

TABLE 8
Biochemical characteristics of B3 antibody

| A. BIACORE | High density surface | Low density surface |
|---|---|---|
| Lipid Chip | 12:0 LPA | 18:0 LPA |
| K_{D} (pM), site 1 (site2) | 61(32) | 1.6 (0.3) |

| B. Competition Lipid Cocktail | |
|---|---|
| (C₁₆:C₁₈:C_{18:1}:C_{18:2}:C_{20:4}, | |
| ratio 3:2:5:11:2) | (μM) |
| IC₅₀ | 0.263 |

C. Neutralization Assay

| B3 antibody (nmol) | LPA (nmol) |
|---|---|
| 0 | 0.16 |
| 0.5 | 0.0428 |
| 1 | 0.0148 |
| 2 | under limit of detection |

A. Biacore analysis for B3 antibody. 12:0 and 18:0 isoforms of LPA were immobilized onto GLC sensor chips; solutions of B3 were passed over the chips and sensograms were obtained for both 12:0 and 18:0 LPA chips. Resulted sensograms showed complex binding kinetics of the antibody due to monovalent and bivalent antibody binding capacities. K_{D} values were calculated approximately for both LPA 12 and LPA 18.
B. Competition ELISA assay was performed with B3 and a cocktail of LPA isoforms (C₁₆:C₁₈:C_{18:1}:C_{18:2}:C_{20:4} in ratio 3:2:5:11:2). Competitor/Cocktail lipid (up to 10 μM) was serially diluted in BSA/PBS and incubated with 0.5 μg/mL B3. Mixtures were then transferred to a LPA coated well plate and the amount of bound antibody was measured. Data were normalized to maximum signal (A₄₅₀) and were expressed as IC₅₀ (half maximum inhibition concentration).
C. Neutralization assay: Increasing concentrations of B3 were conjugated to a gel. Mouse plasma was then activated to increase endogenous levels of LPA. Activated plasma samples were then incubated with the increasing concentrations of the antibody-gel complex. LPA leftover which did not complex to the antibody was then determined by ELISA. LPA was sponged up by B3 in an antibody concentration dependent way.

Selected studies conducted with Lpathomab/LT3000/B7 and B3 are described in Lpath's patent applications e.g., US Patent Application Publication Nos: 20090136483, 20080145360, 20100034814 and 20110076269, all of which are commonly owned with the instant invention and are incorporated herein by reference in their entirety. Briefly, in cancer and angiogenesis models, B7/LT3000 demonstrated:

Inhibition of tumor growth in human tumor xenograft models in vivo;

Reduction in LPA-dependent cell proliferation and invasion of human tumor in vitro;

Reduction in angiogenesis, together with reductions in circulating levels of tumorigenic/angiogenic growth factors including IL6, IL8, GM-CSF, MMP2 in vivo;

Reduction in metastatic potential; and

Neutralization of LPA-induced protection against tumor-cell death.

In In Vitro Models:

Reduction of proliferation of OVCAR3 ovarian cancer cells;

Neutralization of LPA-induced release of IL-8 from Caki-1, IL-8 and IL-6 from SKOV3 (ovarian) tumor cells in vitro;

Mitigation of LPA's effects in protecting SKOV3 tumor cells from apoptosis (which suggests enhanced efficacy when used in combination with standard chemotherapeutic agents);

Inhibition of LPA-induced tumor cell migration and invasion from chemotherapeutic agents.

In In Vivo Models:

Inhibition of metastasis and progression of orthotopic, intraperitoneal and subcutaneous human tumors implanted in nude mice;

Reduction of tumor-associated angiogenesis in subcutaneous SKOV3 xenograft models and in prostate DU145 cancer cells;

Neutralization of bFGF- and VEGF-induced angiogenesis in the murine Matrigel plug assay; and

Reduced choroidal neovascularization in a model of laser-induced injury of Bruch's membrane in the eye.

In fibrosis models, LT3000 reduced inflammation and fibrosis following bleomycin model of pulmonary fibrosis in mice, and was effective both prophylactically and interventionally in this well accepted model. In a diagnostic context, a noninvasive method for detecting fibrosis is a patient sample by correlating LPA levels with levels of one or more fibrogenic markers (e.g., cytokines or growth factors) is believed to be useful for monitoring fibrosis in the clinical setting. It has now been demonstrated that that mice with bleomycin lung injury demonstrated a decrease of IL-13 and TIMP-1 levels, as well as reduction in other relevant growth factors, after treatment with the anti-LPA antibody Lpathomab (LT3000) and consequent reduction in lung fibrosis.

These findings demonstrate a profound role for the bioactive lipid LPA in the extracellular matrix production and tissue remodeling following injury. Furthermore these studies identify LPA as a novel clinical target in treating fibrosis associated with a number of diseases and organ systems. Monoclonal antibodies to LPA are believed to have great clinical potential for treatment of fibrosis.

### Example 8: Humanization of Lpathomab (LT3000)

Humanization of LT3000

The variable domains of the murine anti-LPA monoclonal antibody, LT3000 (Lpathomab) were humanized by grafting the murine CDRs into human framework regions (FR). Lefranc, M. P, (2003). Nucleic Acids Res, 31: 307-10; Martin, A. C. and J. M. Thornton, (1996) J Mol Biol, 1996. 263: 800-15; Morea, V., A. M. Lesk, and A. Tramontano (2000) Methods, 20: 267-79; Foote, J. and G. Winter, (1992) J Mol Biol, 224: 487-99; Chothia, C., et al., (1985). J Mol Biol, 186:651-63. Details of the humanization process are described in US Patent Application Publication 20090136483.

Suitable acceptor human FR sequences were selected from the IMGT and Kabat databases based on a homology to LT3000 using a sequence alignment and analysis program (SR v7.6). Lefranc, M. P. (2003) Nucl. Acids Res. 31:307-310; Kabat, E. A. et al. (1991) Sequences of Proteins of Immunological Interest, NIH National Techn. Inform. Service, pp. 1-3242. Sequences with high identity at FR, vernier, canonical and VH-VL interface residues (VCI) were initially selected. From this subset, sequences with the most non-conservative VCI substitutions, unusual proline or cysteine residues and somatic mutations were excluded. AJ002773 was thus selected as the human framework on which to base the humanized version of LT3000 heavy chain variable domain and DQ187679 was thus selected as the human framework on which to base the humanized version of LT3000 light chain variable domain.

A three-dimensional (3D) model containing the humanized VL and VH sequences was constructed to identify FR residues juxtaposed to residues that form the CDRs. These FR residues potentially influence the CDR loop structure and the ability of the antibody to retain high affinity and specificity for the antigen. Based on this analysis, 6 residues in AJ002773 and 3 residues in DQ187679 were identified, deemed significantly different from LT3000, and considered for mutation back to the murine sequence.

Antibody Expression and Production in Mammalian Cells

The murine antibody genes were cloned from hybridomas. Synthetic genes containing the human framework sequences and the murine CDRs were assembled from synthetic oligonucleotides and cloned into pCR4Blunt-TOPO using blunt restriction sites. After sequencing and observing 100% sequence congruence, the heavy and light chains were cloned and expressed as a full length IgG1 chimeric antibody using the pConGamma vector for the heavy chain gene and pConKappa vector for the light chain gene (Lonza Biologics, Portsmouth N.H.). The expression cassette for each of these genes contained a promoter, a kozak sequence, and a terminator. These plasmids were transformed into E. coli (One Shot Top 10 chemically competent E. coli cells, Invitrogen, Cat No. C4040-10), grown in LB media and stocked in glycerol. Large scale plasmid DNA was prepared as described by the manufacturer (Qiagen, endotoxin-free MAXIPREP™ kit, Cat. No 12362). Plasmids were transfected into the human embryonic kidney cell line 293F using 293fectin and using 293F-FreeStyle Media for culture. The transfected cultures expressed approximately 2-12 mg/L of humanized antibody.

Antibody Purification

Monoclonal antibodies were purified from culture supernatants using protein A affinity chromatography. Aliquots containing 0.5 ml of ProSep-vA-Ultra resin (Millipore, Cat. No 115115827) were added to gravity-flow disposable columns (Pierce, Cat. No 29924) and equilibrated with 10-15 ml of binding buffer (Pierce, Cat. No 21001). Culture supernatants containing transiently expressed humanized antibody were diluted 1:1 with binding buffer and passed over the resin. The antibody retained on the column was washed with 15 ml of binding buffer, eluted with low pH elution buffer (Pierce, Cat. No 21004) and collected in 1 ml fractions containing 100 ul of binding buffer to neutralize the pH. Fractions with absorbance (280 nm)>0.1 were dialyzed overnight (Slide-A-Lyzer Cassettes, 3500 MWCO, Pierce, Cat. No 66382) against 1 liter of PBS buffer (Cellgro, Cat. No 021-030). The dialyzed samples were concentrated using centricon-YM50 (Amicon, Cat. No 4225) concentrators and filtered through 0.22 uM cellulose acetate membranes (Costar, Cat. No 8160). The purity of each preparation was accessed using SDS-PAGE.

SDS-PAGE Electrophoresis

Each antibody sample was diluted to 0.5 ug/ul using gel loading buffer with (reduced) or without (non-reduced) 2-mercaptoethanol (Sigma, Cat. No M-3148). The reduced samples were heated at 95° C. for 5 min while the non-reduced samples were incubated at room temperature. A 4-12% gradient gel (Invitrogen, Cat. No NP0322) was loaded with 2 ug of antibody per lane and ran at 170 volts for 1 hour at room temperature in 1× NuPAGE MOPS SDS running buffer (Invitrogen, Cat. No NP0001). After electrophoresis, the antibodies were fixed by soaking the gel in 50% methanol, 10% acetic acid for ˜10 min. The gel was then washed with 3×200 ml distilled water. Finally, the bands were visualized by staining the gel overnight in GelCode® Blue Stain (Pierce, Cat. No 2490) and destaining with water.

Quantitative ELISA

The antibody titer was determined using a quantitative ELISA. Goat-anti human IgG-Fc antibody (Bethyl A80-104A, 1 mg/ml) was diluted 1:100 in carbonate buffer (100 mM NaHCO₃, 33.6 mM Na₂CO₃, pH 9.5). Plates were coated by incubating 100 ul/well of coating solution (thiolated LPA-BSA conjugate) at 37° C. for 1 hour. The plates were washed 4× with TBS-T (50 mM Tris, 0.14 M NaCl, 0.05% tween-20, pH 8.0) and blocked with 200 ul/well TBS/BSA (50 mM Tris, 0.14 M NaCl, 1% BSA, pH 8.0) for 1 hour at 37° C. Samples and standard were prepared on non-binding plates with enough volume to run in duplicate. The standard was prepared by diluting human reference serum (Bethyl RS10-110; 4 mg/ml) in TBS-T/BSA (50 mM Tris, 0.14 NaCl, 1% BSA, 0.05% Tween-20, pH 8.0) to the following concentrations: 500 ng/ml, 250 ng/ml, 125 ng/ml, 62.5 ng/ml, 31.25 ng/ml, 15.625 ng/ml, 7.8125 ng/ml, and 0.0 ng/ml. Samples were prepared by making appropriate dilutions in TBS-T/BSA, such that the optical density (OD) of the samples fell within the range of the standard; the most linear range being from 125 ng/ml 15.625 ng/ml. After washing the plates 4× with TBS-T, 100 ul of the standard/samples preparation was added to each well and incubated at 37° C. for 1 hour. Next the plates were washed 4× with TBS-T and incubated for 1 hour at 37° C. with 100 ul/well of HRP-goat anti-human IgG antibody (Bethyl A80-104P, 1 mg/ml) diluted 1:150,000 in TBS-T/BSA. The plates were washed 4× with TBS-T and developed using 100 ul/well of TMB substrate chilled to 4° C. After 7 minutes, the reaction was stopped with 1M H₂SO₄ (100 ul/well). The OD was measured at 450 nm, and the data was analyzed using Graphpad Prizm software. The standard curve was fit using a four parameter equation and used to calculate the human IgG content in the samples.

Direct Binding ELISA

The LPA-binding affinities of the humanized antibodies were determined using a direct binding ELISA assay. Microtiter ELISA plates (Costar) were coated overnight with 1.0 ug/ml thiolated C12:0 LPA conjugated to Imject malieimide activated bovine serum albumin (BSA) (Pierce Co.) diluted in 0.1 M carbonate buffer (pH 9.5) at 37° C. for 1 h. Plates were washed with PBS (137 mM NaCl, 2.68 mM KCl, 10.1 mM Na₂HPO₄, 1.76 mM KH₂PO₄; pH 7.4) and blocked with PBS/BSA/tween-20 for 1 hr at room temp or overnight at 4° C. For the primary incubation (1 hr at room temperature), a dilution series of the anti-LPA antibodies (0.4 ug/mL, 0.2 ug/mL, 0.1 ug/mL, 0.05 ug/mL, 0.0125 ug/mL, and 0 ug/mL) was added to the microplate (100 ml per well). Plates were washed and incubated with 100 ul per well of HRP conjugated goat anti-human (H+L) diluted 1:20,000 (Jackson, cat#109-035-003) for 1 hr at room temperature. After washing, the peroxidase was developed with tetramethylbenzidine substrate (Sigma, cat No T0440) and stopped by adding 1 M H₂SO₄. The optical density (OD) was measured at 450 nm using a Thermo Multiskan EX. The EC₅₀ (half-maximal binding concentration) was determined by a least-squares fit of the dose-response curves with a four parameter equation using the Graphpad Prism software.

LPA Competition ELISA

The specificity of the humanized antibody was determined by competition ELISA. Thiolated C18:0 LPA-BSA conjugate coating material was diluted to 0.33 ug/ml with carbonate buffer (100 mM NaHCO3, 33.6 mM Na2CO3, pH 9.5). Plates were coated with 100 ul/well of coating solution and incubated at 37° C. for 1 hour. The plates were washed 4 times with PBS (100 mM Na2HPO4, 20 mM KH2PO4, 27 mM KCl, 1.37 mM NaCl, pH 7.4) and blocked with 150 ul/well of PBS, 1% BSA, 0.1% tween-20 for 1 h at room temperature. The humanized, anti-LPA antibodies were tested against lipid competitors (14:0 LPA (Avanti, Cat. No 857120), 18:1 LPA (Avanti, Cat. No 857130), 18:1 LPC (Avanti, Cat. No 845875), cLPA (Avanti, Cat. No 857328), 18:1 PA (Avanti, Cat. No 840875), PC (Avanti, Cat. No 850454) at 5 uM, 2.5 uM, 1.25 uM, 0.625 uM, and 0.0 uM. The antibody was diluted to 0.5 ug/ml in PBS, 0.1% tween-20 and combined with the lipid samples at a 1:3 ratio of antibody to sample on a non-binding plate. The plates were washed 4 times with PBS and incubated for 1 hour at room temperature with 100 ul/well of the primary antibody/lipid complex. Next the plates were washed 4 times with PBS and incubated for 1 h at room temperature with 100 ul/well of HRP-conjugated goat anti-human antibody diluted 1:20,000 in PBS, 1% BSA, 0.1% tween-20. Again the plates were washed 4 times with PBS and developed using TMB substrate (100 ul/well) at 4° C. After 8 minutes, the reaction was stopped with 100 ul/well of 1M H2SO4. The optical density (OD) was measured at 450 nm using a Thermo Multiskan EX. Raw data were transferred to GraphPad software for analysis.

Thermostability

The thermostability of the humanized antibodies were studied by measuring their LPA-binding affinity (EC50) after heating using the direct binding ELISA. Antibodies dissolved in PBS (Cellgo, Cat. No 021-040) were diluted to 25 ug/ml and incubated at 60° C., 65° C., 70° C., 75° C. and 80° C. for 10 min. Prior to increasing the temperature, 10 ul of each sample was removed and diluted with 90 ul of PBS and stored on ice. The samples were then vortexed briefly and the insoluble material was removed by centrifugation for 1 min at 13,000 rpm. The binding activity of the supernatant was determined using the direct LPA-binding ELISA and compared to a control, which consisted of the same sample without heat treatment.

Surface Plasmon Resonance

All binding data were collected on a ProteOn optical biosensor (BioRad, Hercules Calif.). 12:0 LPA-thiol and 18:0 LPA-thiol were coupled to a maleimide modified GLC sensor chip (Cat. No 176-5011). First, the GLC chip was activated with an equal mixture of sulfo-NHS/EDC for seven minutes followed by a 7 minute blocking step with ethyldiamine. Next sulfo-MBS (Pierce Co., cat #22312) was passed over the surfaces at a concentration of 0.5 mM in HBS running buffer (10 mM HEPES, 150 mM NaCl, 0.005% tween-20, pH 7.4). LPA-thiol was diluted into the HBS running buffer to a concentration of 10, 1 and 0.1 uM and injected for 7 minutes producing 3 different density LPA surfaces (˜100, ˜300 and ˜1400 RU). Next, binding data for the humanized antibodies was collected using a 3-fold dilution series starting with 25 nM as the highest concentration (original stocks were each diluted 1 to 100). Surfaces were regenerated with a 10 second pulse of 100 mM HCl. All data were collected at 25° C. Controls were processed using a reference surface as well as blank injections. The response data from each surface showed complex binding behavior which a likely caused by various degrees of multivalent binding. In order to extract estimates of the binding constants, data from the varying antibody concentrations were globally fit using 1-site and 2-site models. This produced estimates of the affinity for the bivalent (site 1) and monovalent site (site 2).

LPA Molar Binding Capacity

The molar ratio of LPA:mAb was determined using a displacement assay. Borosilicate tubes (Fisherbrand, Cat. No 14-961-26) were coated with 5 nanomoles of biotinylated LPA (50 ug of lipid (Echelon Bioscienes, Cat. No L-012B, Lot No F-66-136 were suspended in 705 ul of 1:1 chloroform:methanol yielding a 100 uM solution) using a dry nitrogen stream. The coated tubes were incubated with 75 ul (125 pmoles) of antibody dissolved in PBS (Cellgro, Cat. No 021-030) at room temperature. After 3 hours of incubation, the LPA:mAb complexes were separated from free lipid using protein desalting columns (Pierce, Cat, No 89849), and the molar concentration of bound biotinylated LPA was determined using the HABA/Avidin displacement assay (Pierce, Cat. No 28010) according to the manufacturer's instructions.

Engineering of the Humanized Variants

The murine anti-LPA antibody was humanized by grafting of the Kabat CDRs from LT3000 V_{H} and V_{L} into acceptor human frameworks. Seven humanized variants were transiently expressed in HEK 293 cells in serum-free conditions, purified and then characterized in a panel of assays. Plasmids containing sequences of each light chain and heavy chain were transfected into mammalian cells for production. After 5 days of culture, the mAb titer was determined using quantitative ELISA. All combinations of the heavy and light chains yielded between 2-12 ug of antibody per ml of cell culture.

Characterization of the Humanized Variants

All the humanized anti-LPA mAb variants exhibited binding affinity in the low picomolar range similar to the chimeric anti-LPA antibody (also known as LT3010) and the murine antibody LT3000. All of the humanized variants exhibited a T_{M} similar to or higher than that of LT3000. With regard to specificity, the humanized variants demonstrated similar specificity profiles to that of LT3000. For example, LT3000 demonstrated no cross-reactivity to lysophosphatidyl choline (LPC), phosphatidic acid (PA), various isoforms of lysophosphatidic acid (14:0 and 18:1 LPA, cyclic phosphatidic acid (cPA), and phosphatidylcholine (PC).

Activity of the Humanized Variants

Five humanized variants were further assessed in in vitro cell assays. LPA is known to play an important role in eliciting the release of interleukin-8 (IL-8) from cancer cells. LT3000 reduced IL-8 release from ovarian cancer cells in a concentration-dependent manner. The humanized variants exhibited a similar reduction of IL-8 release compared to LT3000.

Two humanized variants were also tested for their effect on microvessel density (MVD) in a Matrigel tube formation assay for neovascularization. Both were shown to decrease MVD formation.

### Example 9: Preliminary Animal Pharmacokinetics of Lpathomab

Preliminary PK studies were conducted with Lpathomab. For IV dosed groups, mice were injected with a single 30 mg/kg dose and sacrificed at time points up to 15 days. Antibody was also given via i.p. administration and animals were sacrificed during the first 24 hrs to compare levels of mAb in the blood over this period of time for different routes of delivery. Pharmacokinetic parameters were assessed by WinNonlin. Three mice were sacrificed at each time point and plasma samples were collected and analyzed for mAb levels by ELISA. The half-life of Lpathomab in mice was determined to be 102 hrs (4.25 days) by i.v. administration. Moreover, the antibody is fully distributed to the blood within 6-12 hrs when given i.p., suggesting that the i.p. administration is suitable for xenografts and other studies.

### Example 10: Spinal Cord Injury and Immunohistochemical Staining of LPA Using Monoclonal Antibody to LPA

Immunohistochemical methods can be used to determine the presence and location of LPA in cells. Spinal cords (adult (3 months old) male C57BL/6 mice) from animals with and without spinal cord injury were immunostained 4 days after injury. Adult C57BL/6 mice (20-30 g) were anaesthetized with a mixture of ketamine and xylazine (100 mg/kg and 16 mg/kg, respectively) in phosphate buffered saline (PBS) injected intraperitoneally. The spinal cord was exposed at the low thoracic to high lumbar area, at level T12, corresponding to the level of the lumbar enlargement. Fine forceps were used to remove the spinous process and lamina of the vertebrae and a left hemisection was made at T12. A fine scalpel was used to cut the spinal cord, which was cut a second time to ensure that the lesion was complete, on the left side of the spinal cord, and the overlying muscle and skin were then sutured. This resulted in paralysis of the left hindlimb. After 2 or 4 days the animals were re-anaesthetized as above and then perfused with PBS through the left ventricle of the heart, followed by 4% paraformaldehyde (PFA). After perfusion, the spinal cords were gently removed using fine forceps and post-fixed for 1 hour in cold 4% PFA followed by paraffin embedding or cryo-preserving in 20% sucrose in PBS overnight at 40 C for frozen sections. Tissues for taken from n=3 uninjured mice and n=3 injured mice at 2 and 4 days post-injury. As described in Goldshmit Y, Galea M P, Wise G, Bartlett P F, Turnley A M: Axonal regeneration and lack of astrocytic gliosis in EphA4-deficient mice. J Neurosci 2004, 24(45):10064-10073.

IHC frozen spinal cord sagittal sections (10 μm) were examined using standard immunohistochemical procedures to determine expression and localization of the different LPA receptors. Frozen sections were postfixed for 10 min with 4% PFA and washed 3 times with PBS before blocking for 1 hour at room temperature (RT) in blocking solution containing 5% goat serum (Millipore) and 0.1% Triton-X in PBS in order to block non-specific antisera interactions. Primary antibodies used were B3 (0.1 mg/ml) rabbit anti-LPA1 (1:100, Cayman Chemical, USA), rabbit anti-LPA2 (1:100, Abcam, UK) and mouse anti-GFAP (1:500, Dako, Denmark). Primary antibodies were added in blocking solution and sections incubated over night at 40 C. They were then washed and incubated in secondary antibody for 1 hr at RT, followed by Dapi counterstain. Sections were coverslipped in Fluoromount (Dako) and examined using an Olympus BX60 microscope with a Zeiss Axiocam HRc digital camera and Zeiss Axiovision 3.1 software capture digital images. Some double labeled sections were also examined using a Biorad MRC1024 confocal scanning laser system installed on a Zeiss Axioplan 2 microscope. All images were collated and multi-colored panels produced using Adobe Photoshop 6.0.

After injury, non-neuronal glial cells in the CNS called astrocytes respond to many damage and disease states resulting in a “glial response”. Glial Fibrillary Acidic Protein (GFAP) antibodies are widely used to see the reactive astrocytes which form part of this response, since reactive astrocytes stain much more strongly with GFAP antibodies than normal astrocytes. LPA was revealed by immunohistochemistry using antibody B3 (0.1 mg/ml overnight). Fluorescence microscopy showed that reactive astrocytes are present in spinal cords 4 days after injury, and these cells stain positively for LPA. In contrast, uninjured (control) spinal cords have little to no staining for astrocytes or LPA. Thus LPA is present in reactive astrocytes of the spinal cord. In both injured and control animals, the central canal (hypothesized to be a stem cell niche) does not stain for LPA.

### Example 11: Functional Recovery in Anti-LPA Antibody-Treated Mice Following Spinal Cord Injury (SCI)

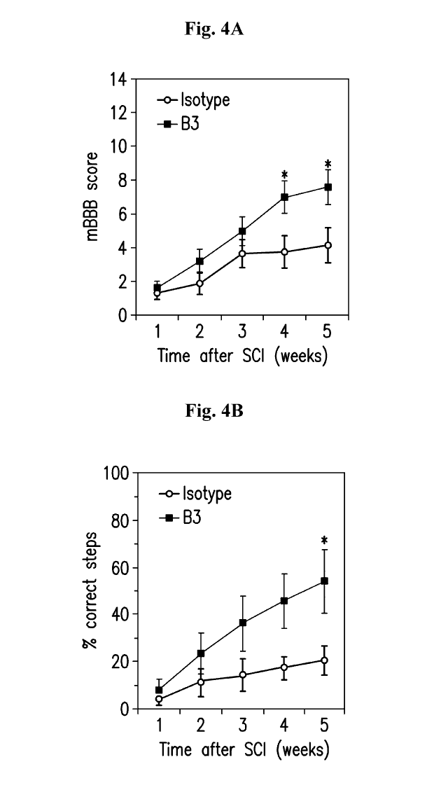

Wildtype mice were given spinal cord hemisection injury as described in Example 10 above. Administration of anti-LPA antibody B3 for two weeks following SCI was found to result in significant functional recovery as determined by open field locomotor test (mBBB) and grid walking test (Goldshmit, et al. (2008), J. Neurotrauma 25(5): 449-465). mBBB is an assessment of hindlimb functional deficits, using a scale ranging from 0, indicating complete paralysis, to 14, indicating normal movement of the hindlimbs. Results are presented as mean+/−SEM. FIG. 4a shows a statistically significant improvement in functional recovery measured by the mBBB at weeks 4 and 5 post-SCI. Mice were also given a grid walking test to assess locomotor function recovery, which combines motor sensory and proprioceptive ability. The test requires accurate limb placement and precise motor control. Intact (uninjured) animals typically cross the grid without making missteps. In contrast, hemisectioned animals make errors with the hindlimb ipsilateral to the lesion. Mice were tested on a horizontal wire grid (1.2×1.2 cm grid spaces, 35×45 cm total area) at weekly intervals following the spinal cord hemisection. Mice were allowed to walk freely around the grid for three minutes during which a minimum time of two minutes of walking was required. When the left hind limb paw protruded entirely through the grid with all toes and heel extending below the wire surface, this was counted as a misstep. The total number of steps taken with the left hindlimb was also counted. The percentage of correct steps was calculated and expressed+/−SEM. As shown in FIG. 4b, mice treated with anti-LPA antibody B3 showed a dramatic improvement in percent of correct steps in the grid walking test; this improvement was statistically significant at five weeks post-SCI.

### Example 12: Antibody to LPA Improves Axonal Regeneration and Neuronal Survival Following Spinal Cord Injury (SCI)

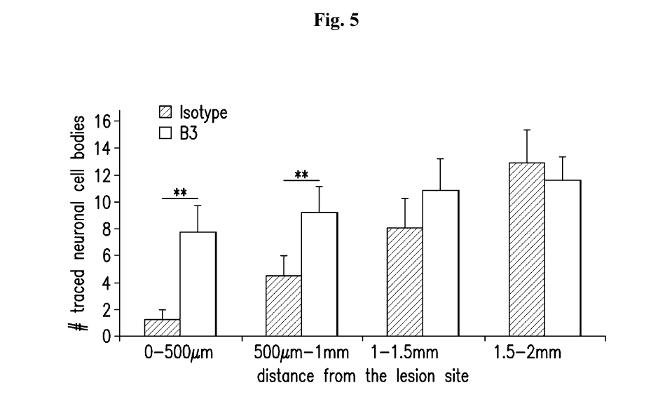

In addition to the functional improvement described in the preceding examples following administration of B3 mAb to wildtype mice for 2 weeks following SCI, anti-LPA antibody treatment also resulted in axonal regeneration through the lesion site and a significant increase in traced neuronal cells that project their processes towards the brain. Tetramethylrhodamine dextran (TMRD) was used to label descending axons that reached the lesion site in isotype controls (n=6) compared to axons that managed to regenerate through the lesion site in B3-treated mice (n=7). Hematoxylin staining was used to reveal the lesion site. Labeled axons also belong to neuronal cells that accumulate label in their cells bodies upstream from the lesion site. Quantitation of number of labeled neuronal cells rostral to lesion site is significantly higher in B3 treated mice (FIG. 5). Data are mean±SEM;**p<0.001. Such neurons may provide later, as part of the plasticity process, a replacement for the loss of long descending or ascending axons after the injury.

### Example 13: Neuroprotective Effects of Anti-LPA Antibody Following SCI

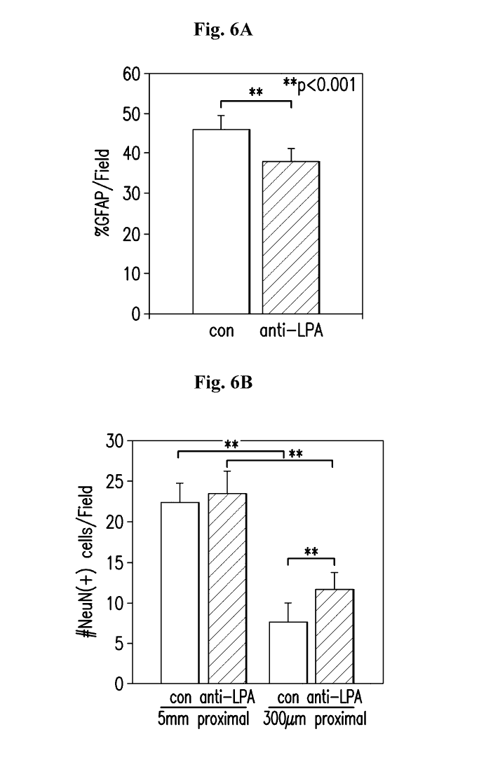

Following SCI as described above, treatment with anti-LPA antibody B3 (0.5 mg/mouse, subcutaneous, twice weekly) for one or two weeks significantly reduces astrocytic gliosis and glial scar formation, as well as neuronal apoptosis. B3 treatment reduces GFAP expression (FIG. 6a) and secretion of chondroitin sulfate proteoglycans (CSPGs), markers for gliosis, into the extracellular matrix by reactive astrocytes at the injury site. Furthermore, B3 antibody treatment also increases neuronal survival at the lesion site, as measured by number of cells staining for NeuN, a neuronal specific nuclear protein (FIG. 6b).

### Example 14: Anti-LPA Antibody in Murine Cortical Impact Model of Traumatic Brain Injury (TBI)—Preventive

The mouse is an ideal model organism for TBI studies because there is an accepted model of human TBI, the type I IFN system in the mouse is similar to that in human, and the ability to generate gene-targeted mice helps to clarify cause and effect rather than mere correlations. Adult mice were anaesthetised with a single ip injection of Ketamine/Xylazine and the scalp above the parietal bones shaved with clippers. Each scalp was disinfected with chlorhexideine solution and an incision made to expose the right parietal bone. A dentist's drill with a fine burr tip was then used to make a 3 mm diameter circular trench of thinned bone centred on the centre of the right parietal bone. Fine forceps were then used to twist and remove the 3 mm plate of parietal bone to expose the parietal cortex underneath. The plate of bone removed was placed into sterile saline and retained. The mouse was mounted in a stereotaxic head frame and the tip of the impactor (2 mm diameter) positioned in the centre of the burr hole at right angles to the surface of the cortex and lowered until it just touches the dura mater membrane covering the cortex. A single impact injury (1.5 mm depth) was applied using the computer controller. The mouse was removed from the head frame and the plate of bone replaced. Bone wax was applied around the edges of the plate to seal and hold the plate in position. The skin incision was then closed with fine silk sutures and the area sprayed with chlorhexidine solution. The mouse was then returned to a holding box underneath a heat lamp and allowed to regain consciousness (total time anaesthetised=30-40 minutes).

### Treatments:

Treatments or isotype controls were injected at various time points. Anti-LPA antibody (B3 or other) was injected by tail-IV (0.5 mg). Following 24-48 hours, the animals were sacrificed and their brains analysed.

### Analysis:

Neuronal death/survival (TUNEL analysis), reactive astrogliosis (revealed by Ki67 positive cells co-labelled with GFAP) and NS/PC responses (proliferation by CD133/Ki67, migration to the injury site by CD133 and differentiation) are analysed. The immune response is assessed by CD11b immunostaining. Quantification is performed by density measurement using ImageJ (NIH).

Results:

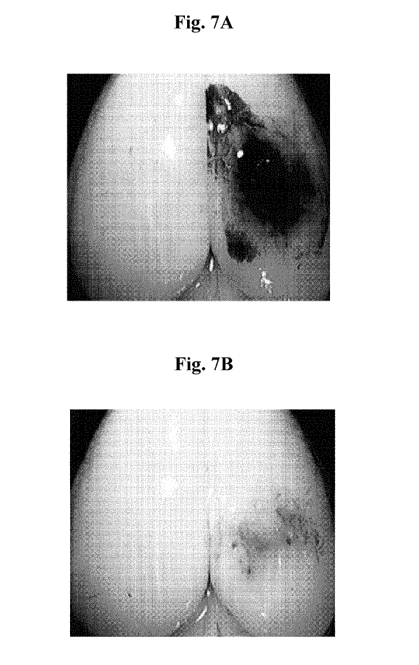

Data from this model show that anti-LPA antibody treatment (B3) administered before injury reduces the degree of hemorrhage normally seen in the mouse brain following TBI in this cortical impact model (FIG. 7).

### Example 15: Anti-LPA Antibodies in Murine Cortical Impact Model of Traumatic Brain Injury (TBI)

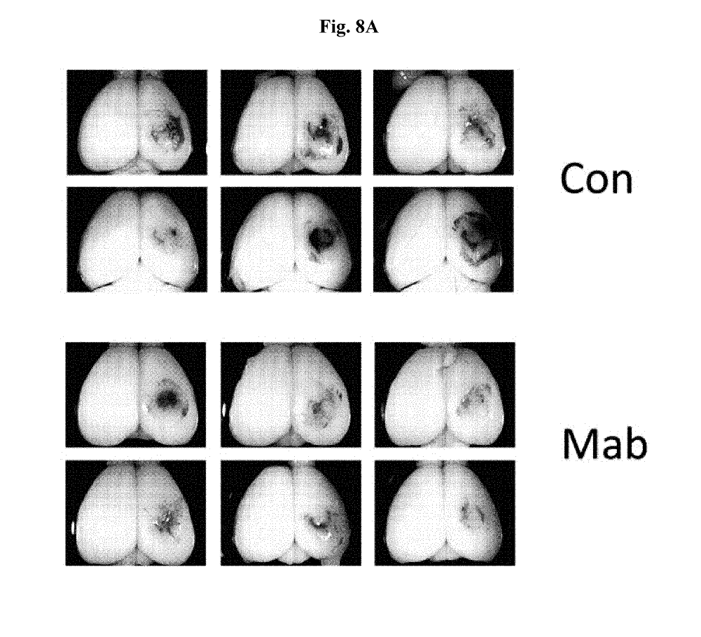

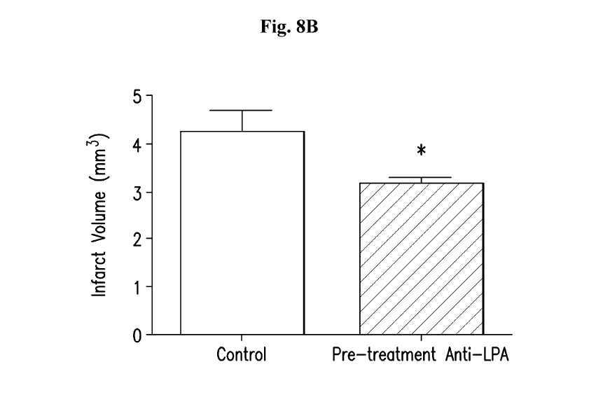

Based on the results of the study described in Example 14, a larger double-blinded prevention study using the same murine cortical impact model was undertaken. Mice were subjected to TBI using Controlled Cortical Impact (CCI) and treated with either isotype control monoclonal antibody or anti-LPA antibody B3 given as a single intravenous dose of 0.5 mg antibody (approx. 25 mg/kg) prior to injury. Mice were sacrificed 24 hours later, at which time the infarct size was photographed and its volume quantified. FIG. 8 shows the histological quantitation of infarct size in anti-LPA treated animals vs. isotype control antibody-treated animals. The reduction in brain infarct volume in animals treated with anti-LPA antibody compared to control animals was statistically significant.

### Example 16: Anti-LPA Antibodies in Murine Cortical Impact Model of Traumatic Brain Injury (TBI)—Interventional Study #1

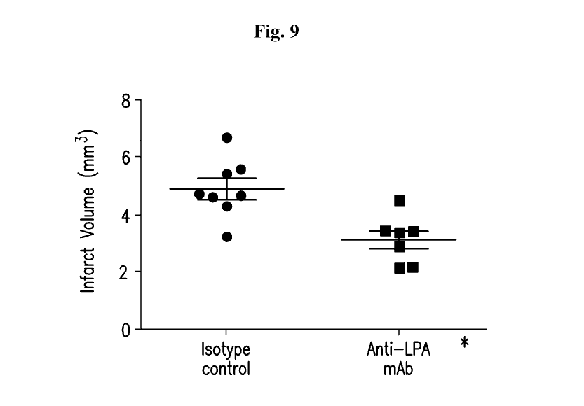

Based on the results of the studies described above, a larger double-blinded interventional treatment study was undertaken using the same clinically relevant murine cortical impact model. Mice (8 animals per group) were subjected to TBI using Controlled Cortical Impact (CCI) and treated with either isotype control monoclonal antibody or anti-LPA antibody B3 given as a single intravenous dose of 0.5 mg antibody (approx. 25 mg/kg) 30 minutes after surgery. Mice were sacrificed 48 hours later, at which time the infarct size was photographed and quantified histologically using image analysis. FIG. 9 shows the histological quantitation of infarct size in each anti-LPA treated animals and each isotype control antibody-treated animal. These data show that treatment with the anti-LPA antibody is neuroprotective for TBI, even when given interventionally (after injury).

### Example 17: Anti-LPA Antibodies in Murine Cortical Impact Model of Traumatic Brain Injury (TBI)—Interventional Study #2

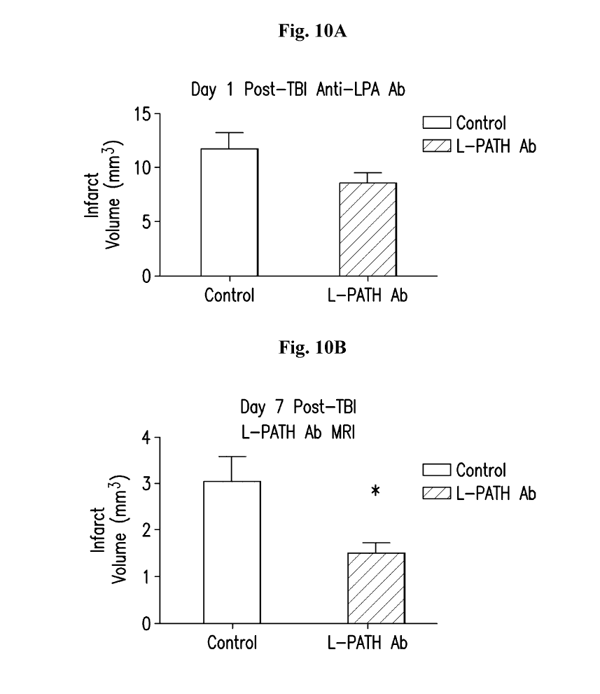

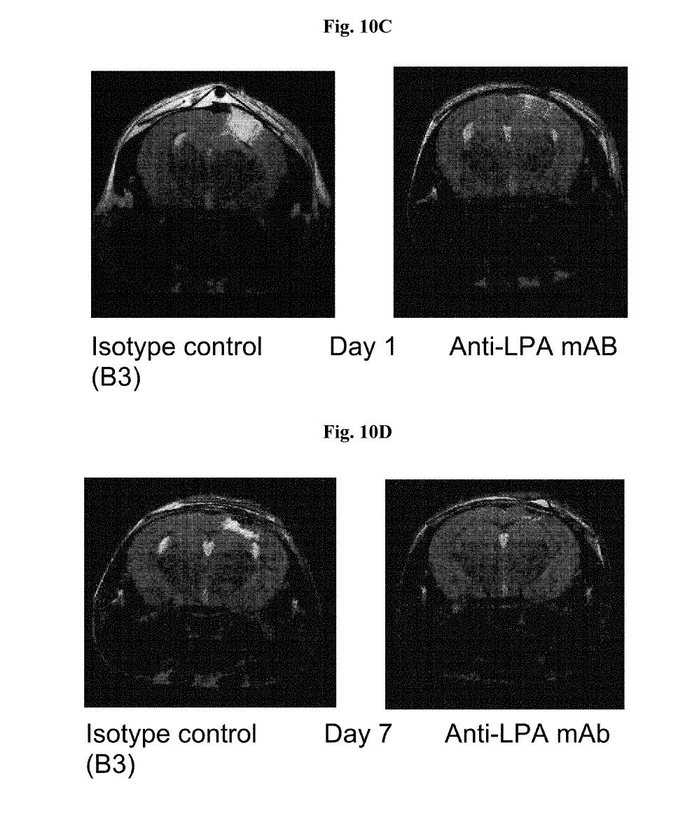

In this double-blinded study, mice (8 per group) were subjected to TBI and treated with an anti-LPA antibody as described in Example 16, but here the mice were sacrificed 7 days after injury. Infarct size was measured by MRI on day 1 and day 7 post-injury, and the results are shown in FIG. 10. These results demonstrate a statistically significant decrease in brain infarct size post-TBI in mice treated with anti-LPA antibody. These data show that treatment with the anti-LPA antibody is neuroprotective for TBI, even when given interventionally after injury. As will be understood, this interventional treatment model is a clinically relevant model.

### Example 18: LPA in Patients' Cerebrospinal Fluid is a Marker for TBI

CSF samples from five TBI patients were obtained from the Neurotrauma Tissue and Fluid Bank, located at the National Trauma Research institute, The Alfred Hospital, Melbourne, Australia, which is part of the Australian Brain Bank Network. CSF was collected from five TBI patients for 5 consecutive days starting at 24 hours after injury (Day 1). Each sample has 2×10 μl and 2×100 μl aliquots=4 tubes per patient. 3× control samples provided, collected at the time of elective neurosurgery. Each control sample has 2×10 μl and 2×100 μl aliquots=4 tubes per subject. Sample information is in Table 9.

TABLE 9
CSF sample information

| Sample name | Day 1 | Day 2 | Day 3 | Day 4 | Day 5 |
|---|---|---|---|---|---|
| 04 | 04_1 | 04_2 | 04_3 | 04_4 | 04_5 |
| 02 | 02_1 | 02_2 | 02_3 | 02_4 | 02_5 |
| 03 | 03_1^{#} | 03_2 | 03_3 | 03_4 | 03_5 |
| 01 | 01_1^{##} | 01_2 | 01_3 | 01_4 | 01_5 |
| 05 | 05_1 | 05_2 | 05_3 | 05_4 | 05_5 |
| Control 1 | | | | | |
| Control 2 | | | | | |
| Control 3 | | | | | |

^{#}samples 03_1, 03_2, 03_3, 03_4, and 03_5 were slightly pale yellow in color
^{##}samples 01_1 were noticeably reddish-pink in color

Patient information is in Table 10. “Admission date” refers to hospital admission. “GCS” refers to Glasgow Coma Score. ISS refers to the Injury Severity Scale. GOSE refers to Extended Glasgow Outcome Scale. Under mechanism of injury, “MVA” refers to motor vehicle accident, “Ped” refers to pedestrian accident, “Pen” refers to penetrating injury.

TABLE 10
TBI patient information

| Patient | | | Admission | Oxygen | Focal/ | Mech of | | | |
|---|---|---|---|---|---|---|---|---|---|
| code | Age | Sex | Date | Saturation | Diffuse | injury | GCS | ISS | GOSE |
| 01 | 23 | M | 5 Mar. 2004 | Non-hypoxic | Focal | Ped | 7 | 33 | 4 |
| 02 | 19 | M | 12 Apr. 2004 | Non-hypoxic | Diffuse | MVA | 8 | 30 | 5 |
| 03 | 50 | M | 15 Apr. 2004 | Hypoxic | Diffuse | MVA | 5 | 41 | 4 |
| 04 | 33 | M | 26 May 2004 | Non-hypoxic | Focal | Ped | 4 | 38 | 1 |
| 05 | 50 | M | 4 Jul. 2005 | Normoxic | | Pen | 10 | 20 | 8 |
| Control 1 | 42 | M | 27 May 2008 | Non-Hypoxic | | | | | |
| Control 3 | 80 | M | 28 Nov. 2007 | Non-Hypoxic | | | | | |
| Control 4 | 56 | M | 17 May 2005 | Non-Hypoxic | | | | | |

The GCS is used to quantitate the severity of coma in a patient who has suffered traumatic brain injury. Mental alertness varies from fully alert to lethargic and stuporous all the way to deep coma, where a patient is minimally responsive or unresponsive to external stimuli. The GCS grades this level of consciousness on a scale from 3 (worst, deep coma) to 15 (normal, alert). A Coma Score of 13 or higher indicates a mild brain injury, 9 to 12 a moderate injury and 8 or less a severe brain injury.

The GOSE is a practical index of outcome or recovery following head injury designed to complement the Glasgow Coma Scale. The eight levels of recovery are: 1) Dead; 2) Vegetative State; 3) Lower Severe Disability; 4) Upper Severe Disability; 5) Lower Moderate Disability; 6) Upper Moderate Disability; 7) Lower Good Recovery; 8) Upper Good Recovery.

The ISS is an anatomical scoring system that provides an overall score for patients with multiple injuries. Each injury is assigned an Abbreviated Injury Scale (AIS) score (from 1 to 6, with 1 being minor, 5 severe and 6 an unsurvivable injury) and is allocated to one of six body regions (Head, Face, Chest, Abdomen, Extremities (including Pelvis), External). Only the highest AIS score in each body region is used. The 3 most severely injured body regions have their score squared and added together to produce the ISS score.

Levels of LPA (multiple lipid species) in CSF samples were measured by high-performance liquid chromatography-electrospray ionization-tandem mass spectrometry (HPLC-ESI-MS/MS) by Professor Andrew Morris at the University of Kentucky under contract with Lpath. The various LPA species in control and injured CSF were determined using published methods. Gellett et al. (2012) BBRC 422:758-763; Federico et al. (2012) Mol. Endocrinol. 26:786-797. While LPA has been detected in CSF (Sato et al., 2005), a detailed analysis by LC-MS identifying the key molecular species of LPA in CSF has not previously been achieved. Numerous LPA species [14:0, 16:1, 16:0, 18:3, 18:2, 18:1, 18:0, 20:4 and 22:6 acyl (ester-linked) LPA] were measured and all physiologically relevant species (16; 0, 18:2, 18:1, 18:0 and 20:4) were detected in the CSF.

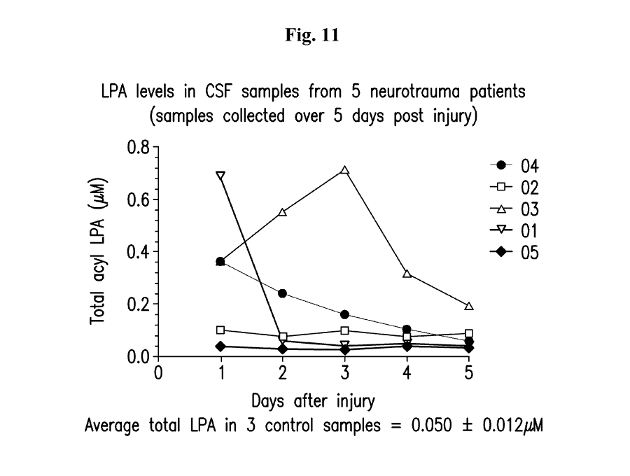

FIG. 11 shows total LPA levels in the CSF of each of 5 TBI patients at 1, 2, 3, 4 and 5 days post-injury. Total LPA levels were elevated in four of the five patients 24 hours after injury and dropped by 48 hours in all but one patient. Interestingly, the patient whose LPA levels did not rise was the patient (05) with a penetrating injury; this patient was the only one with normal (normoxic) oxygen levels and had the highest GCS score (10, indicative of moderate brain injury). The patient (03) whose LPA levels remained high is the only hypoxic patient.

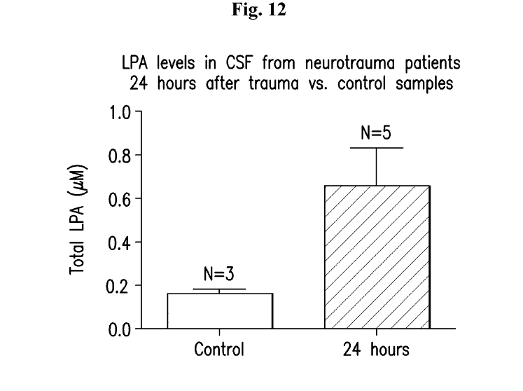

FIG. 12 shows average total LPA levels in the CSF of the same five TBI patients, measured 24 hours after injury, compared to average total LPA in CSF of controls. Thus on average, the total LPA level in the CSF of brain-injured patients is approximately fourfold that of total healthy control subjects.

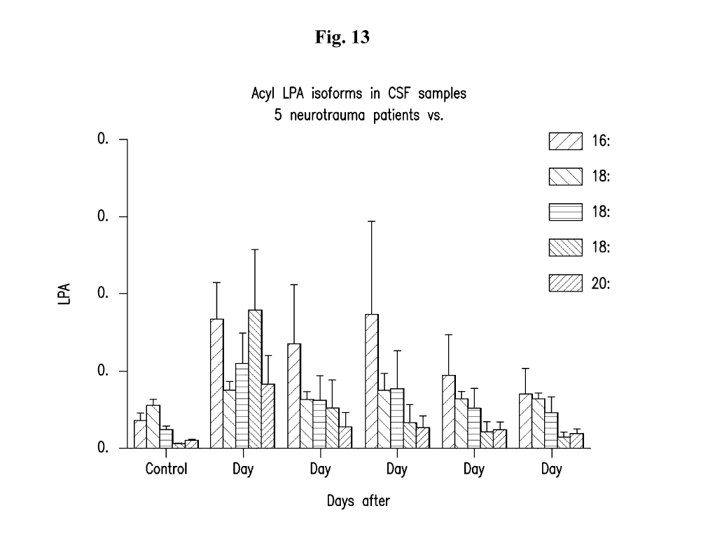

FIG. 13 shows levels of physiologically relevant acyl LPA isoforms in CSF samples from neurotrauma patients at days 1-5 after injury, as well as control subjects. All five of these physiologically relevant isoforms (16:0, 18:0, 18:1, 18:2, 20:4) can be detected in the CSF of injured patients at each time point, as well as in control subjects; however the levels of each isoform are higher in neurotrauma patients compared to control. The difference in LPA levels ranges from a slight increase (in the case of 18:0) to a severalfold increase (16:0, 18:2) over controls. In addition, some isoforms increase dramatically soon after injury and then subside (e.g., 20:4 and 18:2) while other isoforms rise and remain elevated (e.g., 18:0, 18:1). Thus various LPA isoforms may be indicative of different aspects of neurotrauma. Zhao Z, Yu M, Crabb D, Xu Y, Liangpunsakul S. Ethanol-induced alterations in fatty acid-related lipids in serum and tissues in mice. Alcohol Clin Exp Res 2010; 35:229-34.

These preliminary data are the first evidence that LPA levels increase in CSF following neurotrauma. This novel observation that LPA is a biomarker for neurotrauma is the basis for the methods and kits described and claimed herein.

### Example 19 LPA Metabolites in CSF from Patients with TBI

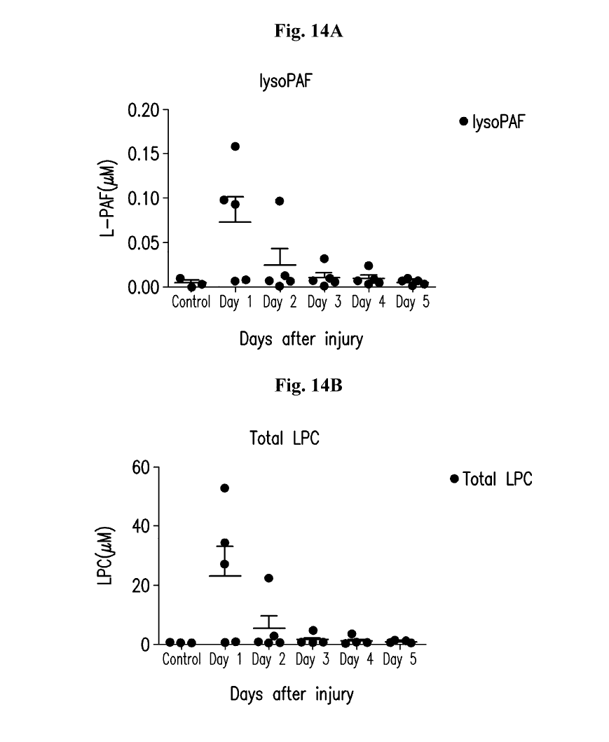

The LPA precursors lyso-PAF and LPC were measured in CSF of TBI patients at 1, 2, 3, 4 and 5 days after injury. Zhao Z, Yu M, Crabb D, Xu Y, Liangpunsakul S. Ethanol-induced alterations in fatty acid-related lipids in serum and tissues in mice. Alcohol Clin Exp Res 2010; 35:229-34. The results are shown in FIG. 14a (lyso-PAF) and 14b (LPC). It can be seen that both LPC and lyso-PAF are highly and significantly elevated at days 1 and 2 after injury, tapering off over days 3 and 4 and returning to approximately control levels by day 5 post-injury. Thus LPA metabolites, including lyso-PAF and LPC, are also believed to be useful biomarkers for TBI. This contrasts with previously published results indicating that lyso-PAF levels do not change after ischemic insult in an animal model of cerebral ischemia. Nishida and Markey (1996) Stroke 27:514-519.

### Example 20 LPA Levels in CSF Samples from Additional Neurotrauma Patients

Further to Example 18, CSF samples from an additional eight TBI patients were obtained. Patient data are shown in Table 11, where available (“-” indicates data not available).

TABLE 11
TBI patient information - second cohort

| | | | | | | | Focal (F) or | |
|---|---|---|---|---|---|---|---|---|
| Patient | | | Mechanism | | | | Diffuse (D) | Hypoxic (Hx) or |
| Number | Age | Sex | of injury | GCS | ISS | GOSE | injury | Normoxic (Nx) |
| 06 | 40 | M | fall/jump | 7 | 21 | 6 | D | Nx |
| 07 | 33 | M | motor bicycle accident | 3 | 43 | 4 | F&D | Hx |
| 08 | 33 | M | fall/jump | 7 | 17 | 6 | F | Nx |
| 09 | 21 | F | motor vehicle accident | 7 | 21 | 3 | D | Nx |
| 10 | 26 | F | fall/jump | 3 | 45 | 3 | F | Hx |
| 11 | 22 | M | penetrating injury | 7 | 30 | 5 | F | Nx |
| 12 | 35 | M | fall/jump | 4 | 45 | 5 | F | Hx |
| 13 | 25 | M | motor vehicle accident | 4 | 41 | — | — | Hx |

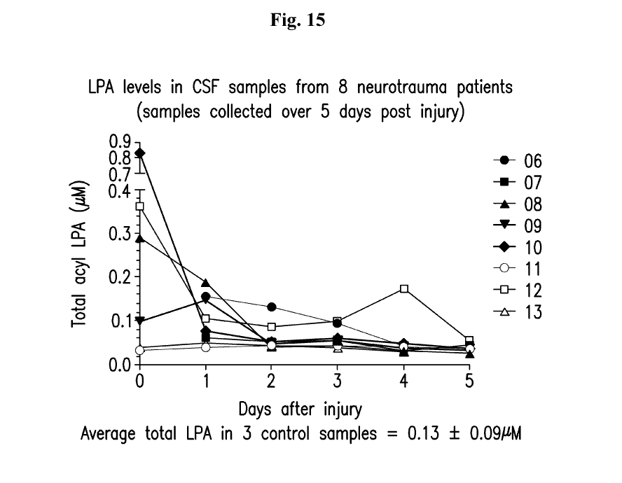

CSF samples were collected over five days post-injury, but collection began earlier after injury than for the five patients in Example 18. FIG. 15 shows total acyl LPA levels in CSF samples from these patients. It can be seen that, in general, LPA levels are highest in the first day after injury, and decrease thereafter.

### Example 21 Time Course of LPA Levels in CSF from Neurotrauma Patients

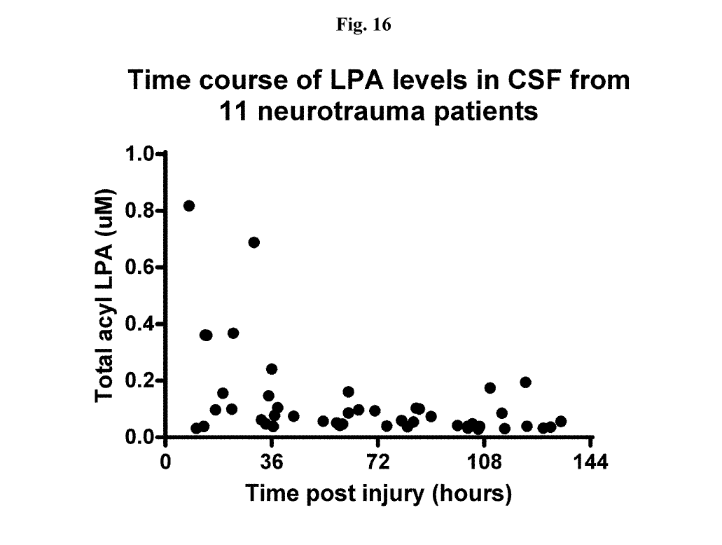

Total acyl-LPA levels were measured in CSF from eleven neurotrauma patients at times up to approximately six days after injury (of the data from the five patients described in Example 18 and the eight patients described in Example 20, data for two patients had to be omitted from the time course due to insufficient information as to time of CSF sampling). Measurements were made by liquid chromatography-mass spectrometry (LC-MS) as described in previous examples. LPA levels were graphed over time in hours after injury as a scatter plot shown in FIG. 16. It can be seen that LPA levels rise dramatically within approximately 36 hours after injury, and particularly in the first half of this time period.

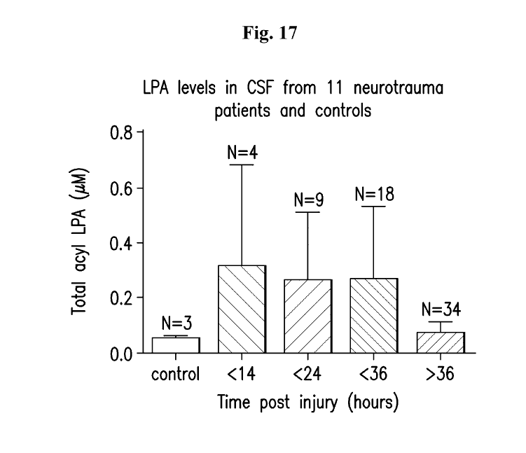

FIG. 17 shows the same measurements grouped by time after injury. It can be seen that total LPA levels are highest within approximately 14 hours of injury, remain high up to about 36 hours post injury and drop to near control levels after about 36 hours post injury. Thus it is believed preferable to use the diagnostic methods and kits disclosed herein, which rely on determining LPA levels in the CSF to detect and/or diagnose neurotrauma, within approximately 36 hours after the injury is sustained.

### Example 22 LPA Isoforms in CSF Samples from 13 Neurotrauma Patients Vs. Controls

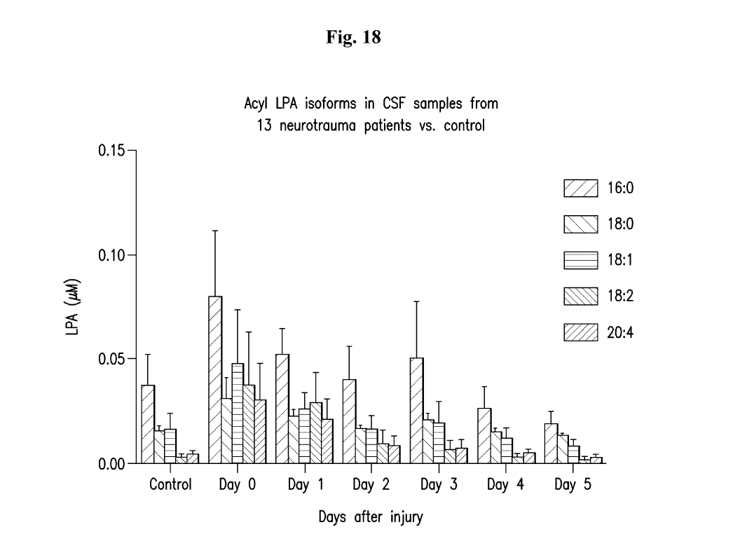

FIG. 18 shows levels of physiologically relevant acyl LPA isoforms in CSF samples from all 13 TBI patients at days 1-5 after injury, as well as control subjects. This includes the data from the five initial patients (Example 18 and FIG. 13) plus the eight additional patients described in Example 20. As before, all five of these physiologically relevant isoforms (16:0, 18:0, 18:1, 18:2, 20:4) were detected in the CSF of injured patients at each time point, as well as in control subjects; however, the levels of each isoform were higher in neurotrauma patients compared to controls.

### Example 23 Correlation of LPA Measurement with Severity of Injury

Because the data above indicate that LPA levels are typically highest in the first 24 hours after neurotrauma, the disease severity scores for the eight patients described in Example 20 were graphed against the LPA levels in the CSF samples taken from these patients within 24 hours post-injury.

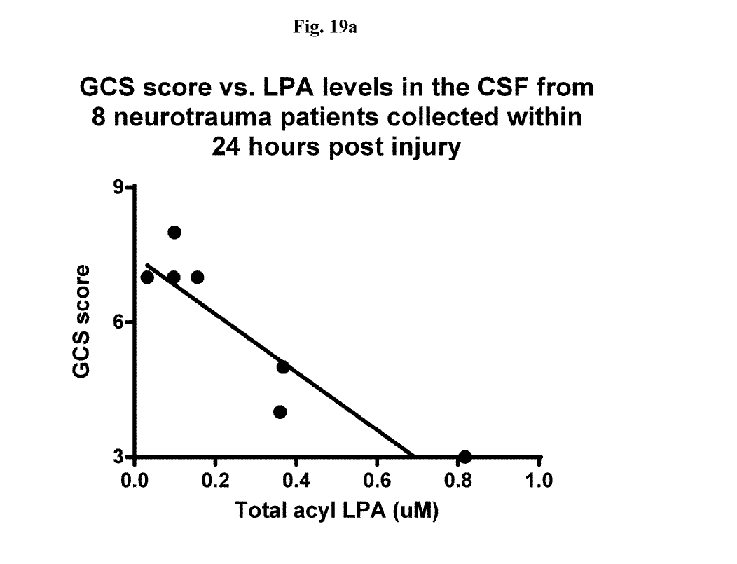

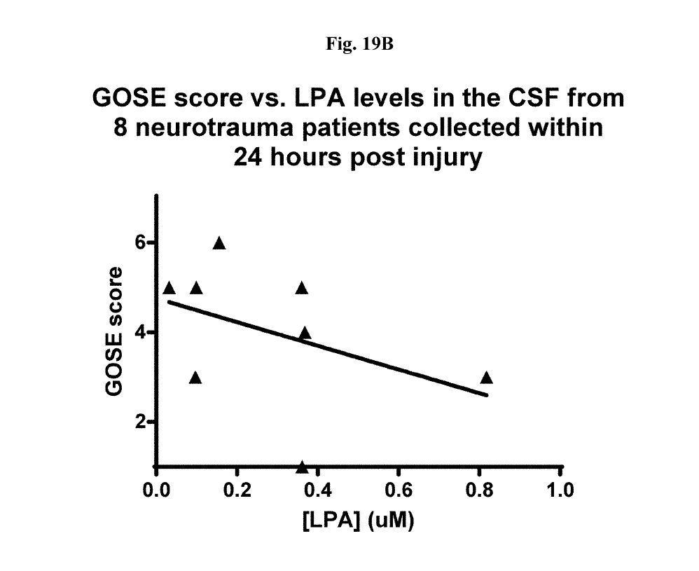

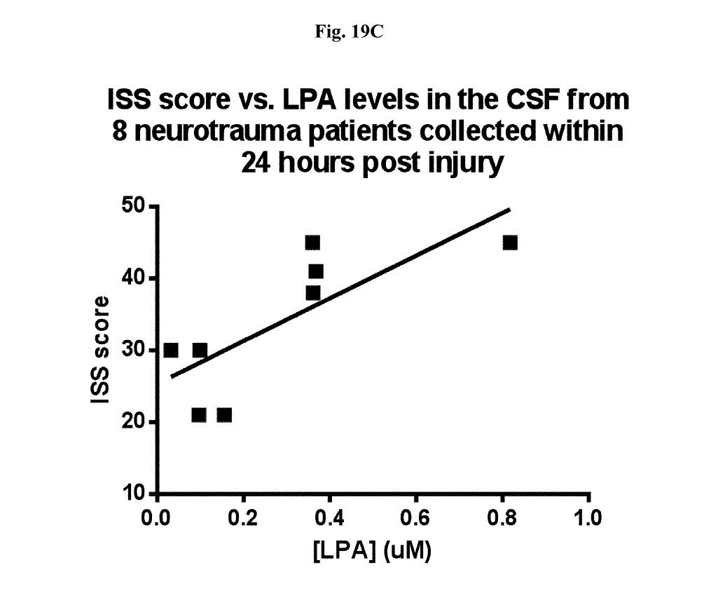

FIG. 19a shows that higher levels of LPA in the CSF are correlated with lower Glasgow Coma Scores (GCS), which indicate a poorer outcome. The GCS is used to quantitate the severity of coma in a patient who has suffered traumatic brain injury on a scale from 3 (worst, deep coma) to 15 (normal, alert). A Coma Score of 13 or higher indicates a mild brain injury, 9 to 12 a moderate injury, and 8 or less a severe brain injury. While all of the patients had GCS scores below 9, indicating a severe brain injury, the highest LPA levels were found in CSF samples from patients with the lowest (most severe injury) scores, and conversely the lowest LPA levels were found in CSF samples from patients with the highest (relatively less severe injury) scores.

FIG. 19b shows that higher levels of LPA in the CSF samples taken from these eight patients within 24 hours of injury are also correlated with lower Extended Glasgow Outcome Scale (GOSE) scores. The GOSE is a practical index of outcome or recovery following head injury designed to complement the Glasgow Coma Scale. The eight levels of recovery are: 1) Dead; 2) Vegetative State; 3) Lower Severe Disability; 4) Upper Severe Disability; 5) Lower Moderate Disability; 6) Upper Moderate Disability; 7) Lower Good Recovery; 8) Upper Good Recovery. Again, the highest LPA levels were found in CSF samples from patients with the lowest (most severe injury) scores, and conversely the lowest LPA levels were found in CSF samples from patients with the highest (relatively less severe injury) scores.

Unlike the GCS and GOSE scoring systems, the Injury Severity Scale(ISS) is an anatomical scoring system that provides an overall score for patients with multiple injuries (polytrauma). Each injury is assigned an Abbreviated Injury Scale (AIS) score (from 1 to 6, with 1 being minor, 5 severe, and 6 an unsurvivable injury) and is allocated to one of six body regions (Head, Face, Chest, Abdomen, Extremities (including Pelvis), External). Only the highest AIS score in each body region is used. The three most severely injured body regions have their scores squared and added together to produce the ISS score. Thus, in contrast to the GCS and GOSE, a low score on the ISS is the most favorable, and a high score is the most severe. As can be seen in FIG. 19c, higher levels of LPA in the CSF samples taken from these eight patients within 24 hours of injury are correlated with higher scores on the ISS (more severe injury).

Thus, it can be seen that, in all three standard scoring methods for neurotrauma and polytrauma, higher levels of LPA in the CSF are correlated with increasing severity of injury, indicating that LPA serves both qualitatively and quantitatively as a biomarker for serious injury such as TBI.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of detecting and treating neurotrauma, if present, in a human subject suspected of having sustained neurotrauma, comprising:
(a) providing a biological sample of tissue or bodily fluid from a human subject suspected of having sustained neurotrauma within about the previous 36 hours;
(b) using an anti-LPA antibody or LPA-binding antibody fragment to determine a level of a first biomarker that is lysophosphatidic acid (LPA) or an LPA metabolite in a biological sample of tissue or bodily fluid from said human subject by contacting the biological sample with the anti-LPA antibody or LPA-binding antibody fragment and detecting a level of binding between LPA and/or an LPA metabolite and the anti-LPA antibody or LPA-binding antibody fragment to determine the level of the first biomarker in the biological sample;
(c) determining a level of said first biomarker that is LPA or an LPA metabolite in a negative control, wherein said determining is performed using an antibody- or antibody fragment-based method;
(d) comparing said level of said first biomarker to the level of LPA or LPA metabolite in said negative control, wherein an elevated level of said first biomarker from said human subject compared to the negative control is indicative of the subject having sustained neurotrauma; and, if neurotrauma is present, and
(e) reducing the effective concentration of LPA in the subject by administering to the subject an anti-LPA antibody or LPA-binding antibody fragment that reduces that effective concentration of LPA in the subject, thereby treating the subject for neurotrauma.

2. A method according to claim 1 wherein the LPA is total LPA, or wherein the LPA is one or more of the group consisting of 16:0 acyl LPA, 18:0 acyl LPA, 18:1 acyl LPA, 18:2 acyl LPA, and 20:4 acyl LPA; and wherein the LPA metabolite is lysophosphatidylcholine (LPC) or lyso-platelet activating factor (lyso-PAF).

3. A method according to claim 1 wherein the antibody- or antibody fragment-based method is an enzyme-linked immunosorbent assay (ELISA) or lateral flow immunoassay.

4. A method according to claim 1 further comprising determining a level of at least one additional protein or lipid biomarker for neurotrauma in said biological sample or in another biological sample from said human subject, wherein the first biomarker and the at least one additional protein or lipid biomarker are not the same, and wherein the first biomarker and the at least one additional protein or lipid biomarker are detected in the same assay or a different assay.

5. A method according to claim 4 wherein the additional protein or lipid biomarker is selected from the group consisting of ubiquitin C-terminal hydrolase (UCH-L1), glial fibrillary acidic protein (GFAP), the phosphorylated form of the high-molecular-weight neurofilament subunit NF-H (pNF-H), LPA, an LPA metabolite and 12-hydroxyeicosatetraenoic acid (12-HETE).

6. A method according to claim 4 wherein if said first biomarker is LPA, said additional biomarker is an LPA metabolite or 12-HETE; wherein if said first biomarker is LPC, said additional biomarker is LPA, lyso-PAF, or 12-HETE; and wherein if said first biomarker is lyso-PAF, said additional biomarker is LPA, LPC, or 12-HETE.

7. A method of claim 1 wherein the first biomarker is LPA and wherein the determining of LPA levels is by an antibody-based method using an antibody which is specifically reactive with LPA, or an LPA-binding fragment thereof.

8. A method according to claim 7 wherein said method further comprises use of a derivatized LPA bound directly or indirectly to a solid support or a carrier moiety, wherein the derivatized LPA is optionally thiolated LPA and the carrier moiety is optionally selected from the group consisting of polyethylene glycol, colloidal gold, adjuvant, a silicone bead, and a protein, optionally wherein the carrier moiety is colored or carries a detectable label.

9. A kit for detecting neurotrauma in a human subject, wherein said kit comprises: an antibody- or antibody fragment-based means for determining a level of a first biomarker that is LPA or an LPA metabolite in a biological sample of tissue or bodily fluid from said human subject; instructions for using the kit, a negative control and an antibody- or antibody fragment-based means for determining a level of said first biomarker that is LPA or an LPA metabolite in said negative control, wherein an elevated level of LPA or an LPA metabolite compared to said negative control is indicative of neurotrauma.

10. A kit according to claim 9 wherein the first biomarker is LPA.

11. A kit according to claim 9 wherein the antibody- or antibody-fragment based means for determining LPA levels is optionally an enzyme-linked immunosorbent assay (ELISA) assay or a lateral flow immunoassay.

12. A kit according to claim 9 wherein the kit further comprises a derivatized LPA that is directly or indirectly bound to a solid support or a carrier moiety, wherein the carrier moiety is optionally selected from the group consisting of polyethylene glycol, colloidal gold, adjuvant, a silicone bead, a latex bead, a colored particle, and a protein.

13. A kit according to claim 9 further comprising a means for determining a level of at least one additional protein or lipid biomarker for neurotrauma in said biological sample or another biological sample from said human subject, wherein the first biomarker and the at least one additional protein or lipid biomarker are not the same and wherein the first biomarker and the at least one additional protein or lipid biomarker are detected in the same assay or a different assay.

14. A kit according to claim 13 wherein the additional protein or lipid biomarker is selected from the group consisting of ubiquitin C-terminal hydrolase (UCH-L1), glial fibrillary acidic protein (GFAP), the phosphorylated form of the high-molecular-weight neurofilament subunit NF-H (pNF-H), LPA, an LPA metabolite, and 12-hydroxyeicosatetraenoic acid (12-HETE).

15. A kit according to claim 9 wherein the LPA metabolite is LPC or lyso-PAF.

16. A kit according to claim 13 wherein if said first biomarker is LPA, said additional biomarker is an LPA metabolite or 12-HETE; wherein if said first biomarker is LPC, said additional biomarker is LPA, lyso-PAF, or 12-HETE; and wherein if said first biomarker is lyso-PAF, said additional biomarker is LPA, LPC, or 12-HETE.

17. A kit according to claim 9 wherein the neurotrauma is selected from the group consisting of traumatic brain injury, spinal cord injury and stroke.

18. A kit according to claim 9 wherein the biological sample of tissue or bodily fluid comprises central nervous system tissue, cerebrospinal fluid (CSF), blood, plasma, or urine.

19. A kit according to claim 9 further comprising a pharmaceutical composition for treating neurotrauma that comprises an agent that reduces the effective concentration of LPA.

20. A kit according to claim 19 wherein the agent that reduces the effective concentration of LPA is a humanized antibody, or fragment thereof, that binds LPA in a tissue or bodily fluid.
